(19) United States
(12) Patent Application Publication
(10) Pub. No.: US 2025/0041338 A1
(43) Pub. Date: Feb. 6, 2025

(54) GENE TARGETS FOR T-CELL-BASED IMMUNOTHERAPY TO OVERCOME SUPPRESSIVE FACTORS

(71) Applicants: The Regents of the University of California, Oakland, CA (US); The J. David Gladstone Institutes, a testamentary trust established under the will of J. David, San Francisco, CA (US)

(72) Inventors: Alexander Marson, San Francisco, CA (US); Eric Shifrut, Oakland, CA (US); Julia Carnevale, Oakland, CA (US); Alan Ashworth, Oakland, CA (US)

(21) Appl. No.: 18/710,157

(22) PCT Filed: Nov. 17, 2022
(86) PCT No.: PCT/US2022/080064

§ 371 Date: May 14, 2024

Related U.S. Application Data
(60) Provisional application No. 63/330,673, filed on Apr. 13, 2022.
(60) Provisional application No. 63/280,487, filed on Nov. 17, 2021.

(51) Int. Cl. A61K 35/17; A61K 39/00; A61P 35/00; C12N 5/0783; C12N 15/90

(52) U.S. Cl. CPC A61K 35/17; A61K 39/4611; A61P 35/00; C12N 5/0636; C12N 15/907; C12N 2510/00

(57) ABSTRACT

Provided herein are genetically modified T cells that exhibit increased proliferation compared to wild-type T cells when stimulated, methods of generating such T cells, and methods of using the T cells for the treatment of a disease such as cancer.

### CROSS-REFERENCE TO RELATED APPLICATIONS

This application is a U.S. National Stage of PCT/US2022/080064, international filing date Nov. 17, 2022, which claims priority benefit of U.S. Provisional Application No. 63/330,673, filed Apr. 13, 2022 and U.S. Provisional Application No. 63/280,487, filed Nov. 17, 2021, each of which is incorporated by reference for all purposes.

### REFERENCE TO AN ELECTRONIC SEQUENCE LISTING

The contents of the electronic sequence listing (081906-1359470-244520PC_ST26.xml; Size: 691,069 bytes; and Date of Creation: Jan. 19, 2023) is herein incorporated by reference in its entirety.

### BACKGROUND OF THE INVENTION

Chimeric antigen receptor (CAR) T cell therapies have been transformative as immunotherapeutics for a subset of aggressive hematological malignancies. In addition, T cell receptor (TCR) transgenic T cells have shown promising results in early phase clinical studies. However, many cancers, especially solid tumors, fail to respond or rapidly progress after initial response to current CAR- or TCR-based T cell therapies. Within the tumor mass, the immunosuppressive microenvironment presents a critical barrier to the efficacy of anti-tumor immunity (see, e.g., Anderson, et al., Cancer Cell 31, 311-325, 2017; Binnewies, et al., Nat. Med. 24, 541-550, 2018). In addition, persistent exposure to antigen can lead to T cell dysfunction, highlighting the need to balance effector function and long-term persistence in engineered T cells (e.g., Vardhana, et al., Nat. Immunol. 21, 1022-1033, 2020; Wei, et al., Nature 576, 471-476, 2019). Targeted manipulation of select genes is being tested as a strategy to boost the efficacy of adoptive T cell therapies. Large-scale CRISPR screens can accelerate the discovery of genetic perturbations that can boost the efficacy of engineered T cells. We previously developed discovery platform in primary human T cells and applied it to identify novel genetic regulators of T cell proliferation (Shifrut, et al., Cell 175, 1958-1971.e15, 2018; WO2020/014235). An adenosine antagonist was employed to simulate KILPATRICK TOWNSEND & STOCKTON LLP elevated adenosine A2A inhibitory signaling in response to high level of adenosine in the hypoxic tumor microenvironment.

### BRIEF SUMMARY OF THE INVENTION

The present disclosure is based, in part, on the development of unbiased genetic screens employing various immunosuppressive conditions commonly encountered in the tumor microenvironment (TME) to identify gene targets that can confer resistance to various forms of suppression found in the tumor microenvironment. To model intrinsic checkpoint signals, we focused on inhibitors of calcium/calcineurin signaling (tacrolimus and cyclosporine) which is a critical pathway for T cell activation often suppressed in tumor infiltrating T cells (Park, et al, Front. Immunol. 11, 195, 2020; Martinez, et al., Immunity 42, 265-278, 2015). To mimic a prominent extrinsic inhibitory signal in the TME, we used TGFβ, a canonical suppressive cytokine limiting T cell function within tumors (Kloss, et al., Mol. Ther. 26, 1855-1866, 2018). Lastly, as regulatory T cells (Tregs) are important mediators of T cell dysfunction in multiple tumor types (Plitas, et al, Immunity 45, 1122-1134, 2016), we adapted our screening platform to assay cell-cell interactions and reveal genes that confer resistance to suppression of effector T cells by Tregs.

Thus, in one aspect, provided herein is a genetically modified hematopoietic cell that comprises a genetic modification to a gene encoding a negative regulator of T-cell stimulation (also referred to herein as T-cell negative regulator gene), e.g., that inhibits expression or activity of the polypeptide product encoded by the gene, wherein expression or activity of the polypeptide product is inhibited by at least 60% compared to a control wild-type hematopoietic cell. In some embodiments, the genetic modification to gene encoding the negative regulator of T-cell stimulation inactivates the gene. In some embodiments, the genetically modified hematopoietic cell is a T cell. In some embodiments, the T cell is a CD8+ T cell or CD4+ T cell. In some embodiments, the T-cell negative regulator gene is inhibited using gene editing technology, for example, a clustered, regularly interspaced, short palindromic repeats (CRISPR) system, including CRISPR interference (CRISPRi), CRISPRoff, and base editing to introduce loss of function mutations. Alternatively, the T-cell negative regulator gene may be inhibited using a transcription activator-like effector nuclease (TALEN) system, a zinc finger nuclease system, or a meganuclease system. In some embodiments, the T-cell negative regulator gene is inhibited using antisense RNA, siRNA, microRNA, or a short hairpin RNA. In some embodiments, the T-cell negative regulator gene that is modified is the gene is selected from the group consisting of ALAS1, AMBRA1, ANKRD32, ARHGAP15, C15orf40, C3orf33, C8orf44, CARKD, CD300LB, CENPB, CHL1, CHST3, CLEC4M, COL15A1, COL25A1, CORO1A, CUL3, CWC27, CYC1, DOK2, DUSP4, ENG, FAM49B, FKBP1A, FUBP1, GAB3, GLRX, GREB1L, GTF2H2, GTF2I, HAUS1, HISTH2AD, HISTIH2BC, HOXA10, IGFBP4, IRF2BP2, IYD, KCNK4, KDM6B, L1CAM, LAMA3, LHX8, MOB3C, MBTD1, MRPL17, MRPL33, MTIF2, MYO1H, NEFL, NFκB1A, NFκB2, NMTJ, ORC6, PCBP2, PCGF1, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNF185, RNF7, RPRD1B, RPS6KL1, SELIL3, SEPW1, SH3BGRL, SIRT7, SIT1, SLC47A1, SLC9A3, SP1, ST5, STAT6, TBL1Y, TGFBR1, TGFBR2, TGIF2, TICRR, TMEM62, TNK1, TNS2, TP53BP1, TTN, TUFM, UPKIB, UQCRC1, WWOX XCL1, ZBTB7A, ZFYVE28, ZNF101, ZNF436, ZNF506, ZNF716, and ZNF805. In some embodiments, the T-cell negative regulator gene is CHST3, TTN, NMTJ, RPS6KL1, STAT6, C8orf44, PDCL, TP53BP1, WWOX GLRX, ZNF506, TNS2, or TBL1Y. In some embodiments, the T-cell negative regulator gene is UQCRC1, IRF2BP2, RPRD1B, AMBRA1, DUSP4, or PCBP2. In some embodiments, the T-cell negative regulator gene is CUL3, CORO1A, RFPLJ, HIST1H2AD, PLGLB2, SH3BGRL, GLRX, ARHGAP15, CHL1, SIT1, CYC1, AMBRA1, GAB3, DOK2, FUBP1, or PDCD6IP. In some embodiments, the T-cell negative regulator gene is KDM6B, COLI5A1, ZFYVE28, CARKD, ZNF101, HOXA10, C3orf33, ALAS1, CYC1, ZBTB7A, FAM49B, MRPL17, GREB1L, PPP2R5D, SLC9A3, CWC27, or GTF2H2. In some embodiments, the T-cell negative regulator gene is ZNF716, XCL1, NFκB2, POTEJ, SP1, NEFL, KCNK4, TNK1, CLEC4M, PCGF1, RNF13, SLC47A1, ZNF436, WWOX ANKRD32, SELIL3, SEPW1, or COL25AL. In some embodiments, the T-cell negative regulator gene is CENPB, CD300LB, IYD, ST5, RNF7, MBTD1, MRPL33, MYO1H, PIWIL4, ZNF805, HISTIH2BC, UPK1B, LAMA3, ENG, ORC6, TICRR, C15orf40, TUFM, RNF185, PTPRG, HAUS1, TMEM62, IGFBP4, L1CAM, or MTIF2.

In a further aspect, provided herein is a population of cells comprising a genetically modified hematopoietic cell, e.g, a T cell, as described herein, e.g., in this paragraph. In some embodiments, a hematopoietic cell, e.g., a T cell, may comprise two or more genetic modifications as described herein.

In a further aspect, provided herein is a method of treating cancer comprising administering a population of cells comprising a genetically modified hematopoietic cell as described herein, e.g., in the preceding paragraph.

In another aspect, provided herein is a genetically modified T cell that has modulated, e.g., reduced, immune function, compared to a control wildtype T cell and comprises a genetic modification to inhibit expression of the polypeptide encoded by the T-cell gene, wherein expression of the polypeptide is inhibited by at least 60% compared to the control wild-type T cell; and the gene is selected from the group consisting of ALAS1, AMBRA1, ANKRD32, ARHGAP15, C15orf40, C3orf33, C8orf44, CARKD, CD300LB, CENPB, CHL1, CHST3, CLEC4M, COL15A1, COL25A1, CORO1A, CUL3, CWC27, CYC1, DOK2, DUSP4, ENG, FAM49B, FKBP1A, FUBP1, GAB3, GLRX GREB1L, GTF2H2, GTF2I, HAUS1, HISTH2AD, HISTIH2BC, HOXA10, IGFBP4, IRF2BP2, IYD, KCNK4, KDM6B, L1CAM, LAMA3, LHX8, MOB3C, MBTD1, MRPL17, MRPL33, MTIF2, MYO1H, NEFL, NFκB1A, NFκB2, NMTJ, ORC6, PCBP2, PCGF1, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNF185, RNF7, RPRD1B, RPS6KL1, SELIL3, SEPW1, SH3BGRL, SIRT7, SIT1, SLC47A1, SLC9A3, SP1, ST5, STAT6, TBL1Y, TGFBR1, TGFBR2, TGIF2, TICRR, TMEM62, TNK1, TNS2, TP53BP1, TTN, TUFM, UPK1B, UQCRC1, WWOX XCL1, ZBTB7A, ZFYVE28, ZNF101, ZNF436, ZNF506, ZNF716, and ZNF805. In some embodiments, the T-cell negative regulator gene is CHST3, TTN, NMT1, RPS6KL1, STAT6, C8orf44, PDCL, TP53BP1, WWOX GLRX, ZNF506, TNS2, or TBL1Y. In some embodiments, the T-cell negative regulator gene is UQCRC1, IRF2BP2, RPRD1B, AMBRA1, DUSP4, or PCBP2. In some embodiments, the T-cell negative regulator gene is CUL3, CORO1A, RFPLJ, HIST1H2AD, PLGLB2, SH3BGRL, GLRX, ARHGAP15, CHL1, SIT1, CYC1, AMBRA1, GAB3, DOK2, FUBP1, or PDCD6IP. In some embodiments, the T-cell negative regulator gene is KDM6B, COLI5A1, ZFYVE28, CARKD, ZNF101, HOXA10, C3orf33, ALAS1, CYC1, ZBTB7A, FAM49B, MRPL17, GREB1L, PPP2R5D, SLC9A3, CWC27, or GTF2H2. In some embodiments, the T-cell negative regulator gene is ZNF716, XCL1, NFκB2, POTEJ, SP1, NEFL, KCNK4, TNK1, CLEC4M, PCGF1, RNF13, SLC47A1, ZNF436, WWOX ANKRD32, SELIL3, SEPW1, or COL25AL. In some embodiments, the T-cell negative regulator gene is CENPB, CD300LB, IYD, ST5, RNF7, MBTD1, MRPL33, MYO1H, PIWIL4, ZNF805, HISTIH2BC, UPK1B, LAMA3, ENG, ORC6, TICRR, C15orf40, TUFM, RNF185, PTPRG, HAUS1, TMEM62, IGFBP4, LICAM, or MTIF2. In some embodiments, the gene is inactivated. In some embodiments, the T cell is a CD8+ or CD4 T cell. In some embodiments, the gene is inhibited using a CRISPR system, a TALEN system, a zinc finger nuclease system, a meganuclease system, an siRNA, an antisense RNA, microRNA, or a short hairpin RNA. In a further aspect, the invention provides a cell culture comprising a genetically modified T cell, e.g., as described herein in this paragraph.

In an additional aspect, provided herein is a method of generating a genetically modified cell population for treatment of a subject that has cancer, the method comprising: obtaining hematopoietic cells from the patient; inhibiting expression of a T-cell negative regulator gene selected from the group consisting of ALAS1, AMBRA1, ANKRD32, ARHGAP15, C15orf40, C3orf33, C8orf44, CARKD, CD300LB, CENPB, CHL1, CHST3, CLEC4M, COL15A1, COL25A1, CORO1A, CUL3, CWC27, CYC1, DOK2, DUSP4, ENG, FAM49B, FKBP1A, FUBP1, GAB3, GLRX GREB1L, GTF2H2, GTF2I, HAUS1, HISTIH2AD, HISTIH2BC, HOXA10, IGFBP4, IRF2BP2, IYD, KCNK4, KDM6B, LICAM, LAMA3, LHX8, MOB3C, MBTD1, MRPL17, MRPL33, MTIF2, MYO1H, NEFL, NFκB1A, NFκB2, NMTJ, ORC6, PCBP2, PCGFJ, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNF185, RNF7, RPRD1B, RPS6KL1, SELIL3, SEPW1, SH3BGRL, SIRT7, SIT1, SLC47AJ, SLC9A3, SP1, ST5, STAT6, TBLIY, TGFBR1, TGFBR2, TGIF2, TICRR, TMEM62, TNKJ, TNS2, TP53BP1, TTN, TUFM, UPKIB, UQCRC1, WWOX XCLJ, ZBTB7A, ZFYVE28, ZNFJ01, ZNF436, ZNF506, ZNF716, and ZNF805; selecting hematopoietic cells in which the T-cell negative regulator gene is inhibited; and expanding the selected hematopoietic cell population ex vivo. In some embodiments, the T-cell negative regulator gene is CHST3, TTN, NMT1, RPS6KL1, STAT6, C8orf44, PDCL, TP53BP1, WWOX GLRX, ZNF506, TNS2, or TBL1Y. In some embodiments, the T-cell negative regulator gene is UQCRCJ, IRF2BP2, RPRD1B, AMBRA1, DUSP4, or PCBP2. In some embodiments, the T-cell negative regulator gene is CUL3, CORO1A, RFPL1, HISTJH2AD, PLGLB2, SH3BGRL, GLRX, ARHGAP15, CHLJ, SIT1, CYC1, AMBRA1, GAB3, DOK2, FUBP1, or PDCD6IP. In some embodiments, the T-cell negative regulator gene is KDM6B, COL15A1, ZFYVE28, CARKD, ZNFJ01, HOXAO, C3orf33, ALAS1, CYC1, ZBTB7A, FAM49B, MRPL17, GREB1L, PPP2R5D, SLC9A3, CWC27, or GTF2H2. In some embodiments, the T-cell negative regulator gene is ZNF716, XCL1, NFκB2, POTEJ, SP1, NEFL, KCNK4, TNK1, CLEC4M, PCGFJ, RNF13, SLC47AJ, ZNF436, WWOX ANKRD32, SELIL3, SEPW1, or COL25AL. In some embodiments, the T-cell negative regulator gene is CENPB, CD300LB, IYD, ST5, RNF7, MBTD1, MRPL33, MYO1H, PIWIL4, ZNF805, HISTIH2BC, UPKIB, LAMA3, ENG, ORC6, TICRR, C15orf40, TUFM, RNF185, PTPRG, HAUS1, TMEM62, IGFBP4, LICAM, or MTIF2. In some embodiments, the hematopoietic cells are hematopoietic stem cells. In some embodiments, the hematopoietic cells are T cells, e.g., CD8+ or CD4+ T cells. In some embodiments, the T-cell negative regulator gene is inhibited using a CRISPR system, a TALEN system, a zinc finger nuclease system, a meganuclease system, an siRNA, an antisense RNA, microRNA, or a short hairpin RNA.

### Definitions

As used herein, the singular forms “a,” “an,” and “the” are also intended to refer to the plural unless the context clearly dictates otherwise.

The terms “polynucleotide” and “nucleic acid” are used interchangeably to refer to a polymeric form of nucleotides of any length, either deoxyribonucleotides or ribonucleotides. The terms include RNA, DNA, and synthetic forms and mixed polymers of the above. In particular embodiments, a nucleotide refers to a ribonucleotide, deoxynucleotide or a modified form or analog of either type of nucleotide, and combinations thereof. In addition, a polynucleotide may include either or both naturally occurring and modified nucleotides linked together by naturally occurring and/or non-naturally occurring nucleotide linkages. The nucleic acid molecules may be modified chemically or biochemically or may contain non-natural or derivatized nucleotide bases. Such modifications include, for example, labels, methylation, substitution of one or more of the naturally occurring nucleotides with an analogue, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). “Polynucleotide” and “nucleic acid” are also intended to include any topological conformation, including single-stranded, double-stranded, partially duplexed, triplex, hairpinned, circular and padlocked conformations. A reference to a nucleic acid sequence encompasses its complement unless otherwise specified. Thus, a reference to a nucleic acid molecule having a particular sequence should be understood to encompass its complementary strand, with its complementary sequence. Reference to a “polynucleotide” or “nucleic acid” that encodes a polypeptide sequence also includes codon-optimized nucleic acids and nucleic acids that comprise alternative codons that encode the same polypeptide sequence.

As used herein, the term “complementary” or “complementarity” refers to specific base pairing between nucleotides or nucleic acids. Base pairing may be perfectly complementary or partially complementary.

The term “gene” can refer to the segment of DNA involved in producing or encoding a polypeptide chain. It may include regions preceding and following the coding region (leader and trailer) as well as intervening sequences (introns) between individual coding segments (exons). Genes are defined by symbol and nomenclature for the human gene as assigned by the HUGO Gene Nomenclature Committee.

A “promoter” is defined as one or more a nucleic acid control sequences that direct transcription of a nucleic acid. As used herein, a promoter includes necessary nucleic acid sequences near the start site of transcription. A promoter also optionally includes distal enhancer or repressor elements, which can be located as much as several thousand base pairs from the start site of transcription.

The term “inhibiting expression” refers to inhibiting or reducing the expression of a gene or a protein. To inhibit or reduce the expression of a gene (i.e., a gene encoding a transcription factor, or a gene regulated by a transcription factor), the sequence and/or structure of the gene may be modified such that the gene would not be transcribed (for DNA) or translated (for RNA), or would not be transcribe or translated to produce a functional protein (e.g., a transcription factor). Various methods for inhibiting or reducing expression of a gene are described in detail further herein. Some methods may introduce nucleic acid substitutions, additions, and/or deletions into the wild-type gene. Some methods may also introduce single or double strand breaks into the gene. To inhibit or reduce the expression of a protein (e.g., a T-cell inhibitory protein), one may inhibit or reduce the expression of the gene or polynucleotide encoding the protein, as described above. In other embodiments, one may target the protein directly to inhibit or reduce the protein's expression using, e.g., an antibody or a protease. “Inhibited” expression refers to a decrease by at least 10% as compared to a reference control level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample). As used herein, the term “inactivated” refers to preventing expression of a polypeptide product encoded by the gene. Inactivation can occur at any stage or process of gene expression, including, but not limited to, transcription, translation, and protein expression, and inactivation can affect any gene or gene product including, but not limited to, DNA, RNA, such a mRNA, and polypeptides. In some embodiments, “inhibited expression” reflects inactivation in a percentage of cells that are modified, e.g., at least about 20%, or at least about 30%, or at least about 40%, or at least about 50%, or at least about 60%, or at least about 70%, or at least about 80%, or at least about 90% or greater of the cells in a population that also comprises cells in which the target gene is not inactivated.

The term “genetic modification” as used herein refers to any modification to a cell to alter expression of a gene. Such modifications include modifications to the genome as well as modifications to introduce inhibitory sequences, such as inhibitory RNAs, into the cell.

As used herein, the phrase “modifying” in the context of modifying a genome of a cell refers to inducing a structural change in the sequence of the genome at a target genomic region. For example, the modifying can take the form of inserting a nucleotide sequence into the genome of the cell. For example, a nucleotide sequence encoding a polypeptide can be inserted into the genomic sequence encoding an endogenous cell surface protein in the T cell. The nucleotide sequence can encode a functional domain or a functional fragment thereof. Such modifying can be performed, for example, by inducing a double stranded break within a target genomic region, or a pair of single stranded nicks on opposite strands and flanking the target genomic region. Methods for inducing single or double stranded breaks at or within a target genomic region include the use of a nuclease domain, e.g., Cas9, or a derivative thereof, and a guide, e.g., guide RNA, directed to the target genomic region.

The terms “patient,” “subject,” “individual,” and the like are used interchangeably herein, and refer to any animal, e.g., a mammal, such as a primate. In certain non-limiting embodiments, the patient, subject or individual is a human.

The terms “treatment”, “treating”, and the like are used herein to generally mean obtaining a desired pharmacologic and/or physiologic effect. The effect may be prophylactic, in terms of completely or partially preventing a disease, condition, or symptoms thereof, and/or may be therapeutic in terms of a partial or complete cure for a disease or condition and/or an adverse effect, such as a symptom, attributable to the disease or condition. “Treatment” as used herein covers any treatment of a disease or condition of a subject and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

### BRIEF DESCRIPTION OF THE FIGURES

FIGS. 1A-1H: Multiple genome-wide CRISPR screens in primary human T cells nominate RASA2 as a modulator of resistance to immunosuppressive conditions. a, Schematic of genome-wide screens to discover resistance gene targets in human T cells. b, Shared hits (z-score>1.5) across all screens performed. Bar height is the number of shared hits among the screens, which are connected by dots in the lower panel. (c,d) Log2 fold-change (LFC, x-axis) for individual guides (vertical lines); background shows the overall guide distribution in each condition in grayscale. c, Guides targeting RASA2 (pink lines) were enriched in dividing cells across all suppressive conditions. d, Multiple distinct guides targeting RASA2 were enriched in dividing cells in the TCR-stimulation—screen across biological replicates (n=4 human donors). Guides targeting other members of the RasGAP family were not enriched consistently in either direction, while guides targeting the RasGEF RASGRP1 were depleted from dividing cells as expected. e, Proliferation assay to validate that RASA2 ablation confers resistance to adenosine, cyclosporine, tacrolimus and TGFβ. CFSE distributions show that RASA2 ablation promoted stronger proliferation compared to control-editing (CTRL) across all suppressive conditions tested. f, Plot of cancer cell growth during in vitro cancer killing assay under suppressive conditions. T cells expressing a TCR specific for an NY-ESO-1 tumor antigen showed better control of cancer cell growth when RASA2 is ablated, as measured by live-cell microscopy of co-cultures with addition of the different inhibitors. AUC is the area under the growth curve of cancer cells (n=2 donors, mean+/−SEM, *p<0.05, **p<0.01 and ***p<0.001 for two tailed paired Student's t-test). g, Suppression assay confirms that RASA2 ablation renders T cells resistant to Treg suppression of proliferation. Effector CD8 T cells were stimulated with anti-CD3/CD28 and co-cultured with donor-matched Tregs in different Treg: CD8 ratios. Bars show the CD8 cell count as measured by flow cytometry 4 days after stimulation (CTRL indicates non-targeting guides, n=4 donors per group, mean±SEM, **p<0.01 and ***p<0.001 for two-tailed paired Student's t-test) h, RASA2 ablation rendered T cells resistant to Treg suppression in a cancer killing assay compared to control-edited T cells (CTRL) for one representative donor, shaded area is 95% confidence interval for 3 technical replicates.

FIGS. 2A-2D: Multiple genome-wide CRISPR screens for T cell resistance. a, Shared hits (y-axis) (z-score>1.5, methods) across the screen conditions (x-axis) including hits unique to each individual screen. b, Heatmap of the pairwise Pearson's correlation coefficient for gene-level z-scores for all screen conditions. c, Volcano plots showing p-value (MAGeCK RRA test and methods) on the y-axis and gene level z-scores on the x-axis. Highlighted are RASA2 and TMEM222 in each screen, as well as ADORA2A, TGFBR1 and PPIA (cyclosporine binding protein) in their specific suppressive conditions: adenosine, TGFβ and cyclosporine respectively. Vertical line shows the threshold for z-score used to determine intersected hits. d, Log fold change (LFC) of guides targeting RasGAP genes and the RasGEF RASGRP1, across the different suppressive screen conditions shown here (n=4 donors for Stim and Tregs screen, n=2 for Adenosine, Cyclosporine and Tacrolimus and n=1 for the TGFβ screen.

FIGS. 3A-30: RASA2 ablation promotes T cell activation, effector function, and increases sensitivity to antigen. a, Diagram of Ras signaling and downstream transcriptional programs in T cells. b, Left: Western blot showing efficient RASA2 ablation in Jurkat cells, Vinculin (Vinc) as loading control. Right: GTP-bound active Ras in Jurkat cells after TCR-stimulation. c, Scaled mean fluorescent intensity (MFI) by flow cytometry for phospho-proteins in the MAPK and Akt/mTOR pathways (Lines show the mean, n=2 donors in triplicates, **p<0.01 for Wilcoxon test) d, Kinetics of phosphorylated ERK in primary human T cells as measured by flow cytometry (n=2 donors in triplicates, mean±SEM, **p<0.01 for Wilcoxon test). e, Effector cytokine levels were measured using intracellular staining and flow cytometry of stimulated T cells (n=2 donors in triplicates, mean±SEM, *p<0.05 and **p<0.01 for Wilcoxon test). f,g Phosphorylated ERK levels (y-axis) measured by flow cytometry 10 minutes after TCR stimulation with titrated concentrations (log 2(μl/ml)) of anti-CD3/CD28 complexes (f) or T2 cells preloaded with titrated concentrations of the cognate NY-ESO-1 peptide (g). Dots are individual donors (n=2), lines are a fitted 4-parameter dose-response curve. h, Left column: CD19 levels on engineered Nalm6 cancer target cells were measured using flow cytometry (Green) and compared to unstained cells (Grey). Right column: CAR T cell killing of target Nalm6 cells expressing varying CD19 levels measured by annexin levels with live cell microscopy, mean±SEM for technical duplicates from one representative donor. i, Percent of Jurkat cells positive for each mCherry reporter responsive to the transcription factor, as indicated above each panel. Error bars are SEM for triplicates. j, Gene set enrichment analysis of differentially expressed genes between RASA2 and control-edited T cells 48 hours after TCR-stimulation. Normalized enrichment score (NES) is shown at the x-axis indicating the direction of change in expression, size of each dot is the p-value (permutation test). k, Differentially expressed genes (y-axis) in stimulated RASA2 KO T cells from a published single-cell RNA-Seq data set. Circle color is the mean expression and its size is the percentage of cells in which the gene transcript was detected. Data is shown for four different target gene perturbations (x-axis), aggregated across two donors. l-o, RASA2 expression in published datasets. 1, Mouse model of Listeria infection (n=3 mice, mean SEM) m, In vitro activated human T cells (n=91 donors, lines are mean, dots are individual donors, ****p<0.0001 for Wilcoxon test). n, Mouse model of tumor-infiltrating T cells (TIL), x-axis shows days after T cell transfer (n=3 mice, mean±SEM). o, RASA2 expression (transcripts per million, TPM, y-axis) in human patient tumor-infiltrating (orange) or peripheral T cells (green). Each dot is an individual cell, box shows the upper and lower quartiles, horizontal line is median (n=12 donors for Colorectal cancer (CRC) and n=14 donors for Non-small cell lung carcinoma (NSCLC), exact p-values for Wilcoxon test are shown at the bottom).

FIGS. 4A-4M: RASA2 ablation improves functional T cell persistence through repeated cancer target exposure. a, Experimental system for measuring functional T cell persistence in vitro. b, T cells were stimulated by repeated co-cultures with target cancer cells every 48 hours. T cell viability and CD39 levels were measured by flow cytometry after each stimulation point (n=4 donors, mean±SEM). c, Expression of TOX and GZMB in T cells by RNA-Seq, after the first and fifth stimulation with target cells. (n=4, lines connect individual donors). d, Gene set enrichment analysis of differentially expressed genes between T cells after the first and fifth stimulation shows depletion of oxidative phosphorylation genes following the repetitive stimulation. Adjusted p-value (padj) is by permutation test. e, Co-culture of TCR-T cells with target cancer cells show gradual failure of T cells to control cancer cell growth following multiple stimulations. f, Exhaustion markers as measured by flow cytometry of T cells after multiple stimulations show similar levels between RASA2 KO and control-edited (CTRL) T cells (n=4 donors, mean±SEM, *p<0.05 and ns is p>0.05 for Wilcoxon test). g, RASA2 KO T cells following multiple stimulations show higher levels of phosphorylated ERK and CD69 compared to control cells (n=2 donors). h, Effector cytokine production in T cells after repeated stimulation, as measured by flow cytometry, across both CAR and TCR-T cells (n=2 for TCR, mean±SEM, *p<0.05, **p<0.01 and ***p<0.001 for Wilcoxon test). i, Multiplex ELISA to measure cytokines in the supernatant of T cells after multiple stimulations (n=4 human donors and technical duplicates as dots, lines show mean cytokine levels, *p<0.05 and **p<0.01 for Wilcoxon test). j, Cancer cell killing assay shows control-edited TCR-T cells fail to control cancer cell growth after five stimulations, while RASA2 ablation leads to robust persistent killing capability. k, Imaging of co-culture wells after T cells were exposed to repeated stimulations, where remaining cancer cells are visualized by their RFP nuclear tag. Scale bar is 1 mm. 1, Summary statistics of assays in (j), across 7 human donors and a range of effector T cells to target cell ratios. AUC is the area under the growth curve of cancer cells. Lines show the mean±SEM (n=7 human donors). m, CAR-T cells with RASA2 ablated maintained efficient cancer cell killing despite six prior stimulations with target cells.

FIGS. 5A-5L: RASA2 ablation improves in vivo tumor control by engineered T cells in multiple preclinical models. (a,b) 1×10⁶ NY-ESO-1+A375 melanoma cells were engrafted into NSG mice via flank injection and 1×10⁶ NY-ESO-1-specific 1G4 TCR-T cells were injected via the tail-vein (TV). Mice were monitored for tumor growth by caliper measurements. Mice receiving RASA2 KO T cells showed a reduction in tumor burden (n=6 mice per group, mean±SEM, *p<0.05 for two-tailed paired Student's t-test). (c,d) 0.3×10⁶ Nalm6 leukemia cells engineered to express NY-ESO-1 were injected into NSG mice and tumor load was measured using luciferase-based bioluminescence. 0.5×10⁶ RASA2-KO NY-ESO-1-specific 1G4 TCR-T cells were injected per mouse and reduced the tumor burden compared to control locus edited TCR-T cells (n=5 mice with RASA2 KO T cells, n=4 for CTRL T cells, mean±SEM, *p<0.05 for two-tailed paired Student's t-test). (e,f) 0.5×10⁶ Nalm6 cells were engrafted into NSG mice and 0.2×10⁶ CD19-specific CAR T cells were injected via the tail-vein. Mice were monitored for tumor growth by luciferase-based bioluminescence. Mice receiving RASA2 KO T cells showed a reduction in tumor burden (n=7 mice per group, mean±SEM, ****p<0.0001 for two-tailed paired Student's t-test) g, Bioluminescence images of the cohort in (f), dorsal view. h, Survival curves for the leukemia model of the cohort shown in (f). P-value by log rank test (i-l). i, Scheme of intraperitoneal (IP) LM7 model. NSG mice were injected IP with 1×10⁶ LM7-ffLuc tumor cells on Day 0, and 7 days later received a single IP dose of 1×10⁵ Ctrl or RASA2 KO EphA2-CAR T-cells. j, Quantitative bioluminescence imaging (mean±SEM, n=10 for CTRL, n=14 for RASA2, *p<0.05 for two-tailed paired Student's t-test). k, Representative images of each treatment group. 1, Survival curve for the LM7 cohort in (j) (n=10 for CTRL; n=14 for RASA2 group; exact p-value by log-rank test).

FIG. 6: Gene targets from screens selected as either general (PAN) or specific to certain suppressive contexts and were individually knocked out in T cells. CFSE stained, edited T cells were stimulated and cultured in the different suppressive conditions. Percent of cells proliferating for each gene compared to control cells are displayed for each suppressive condition (n=2 donors, 2 sgRNAs per gene target in triplicates. Highlighted are genes found to have a significant resistance role in each condition, using a cut-off of FDR adjusted p-value<0.05.

FIGS. 7A07B: Validation of cancer cell killing activity and TCR-T cell proliferation activity. Primary T cells were transduced with the NY-ESO1 TCR and CRISPR-edited for each gene listed (PFN1, PDE4C, RASA2, CBLB, GTF2, TGIF2 and safe harbor control locus AAV). Two different sgRNAs were employed for the majority of the target genes. The edited cells were then co-cultured with A375 cancer cells expressing the cognate peptide on matched MHCI. Cancer cell killing (FIG. 7A) was measured with the Incucyte live-cell imaging system. Order of lines, top to bottom at 120 hour time point: AAVS1 control, PFN1_g2, PDE4C_g1, PFN1_g1, TGIF2_g2, GTF2_g2, GTF2_g1, TGIF2_g1, CBLB_g1, PDE4C_g2, RASA2_g1. The TCR-T cells were also tested for their proliferative capacity in response to stimulation (FIG. 7B). data points for each gene, left to right; RASA2_g1, AAVS1_g1 control, CBLB_g1 CBLB_g2, PFN_g1, PFN_g2, PDE4C-g1, PDE4C_g2, GTF2i_g1, GTF2i_g2, TGIF2_g1, TGIF2_g2.

### DETAILED DESCRIPTION OF THE DISCLOSURE

In one aspect, the disclosure provides engineered T cells that exhibit enhanced cytotoxicity to cells, e.g., tumor cells compared to counterpart unmodified T cells. Such engineered T cells are modified to inhibit expression or activity of a T-cell gene that negatively affects proliferation, e.g., in a tumor microenvironment, i.e., is a negative regulator of T cell stimulation. Such a gene is referred to herein as a T-cell negative regulator gene. In some embodiments, inhibition of such a gene confers resistance to immunosuppressive signals, such as, but not limited to: TGFβ, high levels of adenosine found in a hypoxic tumor microenviron, and/or suppressed calcium/calcineurin signaling. In some embodiments, inhibition of a T-cell gene, e.g., an effector T cell, confers resistance to suppression by regulatory T cells (Tregs).

Modification to T-cell negative regulator gene in accordance with the invention need not be limited to modification of the gene in a T cell. Although the modifications are T-cell receptor (TCR) dependent, they can be applied to other hematopoietic cells. Thus, in some embodiments, a cell modified in accordance with the invention is a T cell, such as a CD8+ T cell. In some embodiments, the cell is a hematopoietic stem cell. In further embodiments, the cell is a stem memory T cell, an effector memory T cell, a central memory T cell, or a naïve T cell. In some embodiments, modifications in accordance with the invention are made to CD4+ T cells, or NK cells or gamma delta T cells. Review of T cell subsets are provided, e.g., in Sallusto et al., Annual Rev. Immunol. 22745-763, 2004; Mueller et al., Annual Rev. Immunol 31:137-161, 2013; and for memory stem T-cells, Gattinoni, et al., Nature Med. 23:18-27, 2018. Descriptions of subsets by markers are available in the OMIP Wiley Online Library (see, e.g., Wingender and Kronenberg, OMIP-030: Characterization of human T cell subsets via surface markers Cytometry Part A 87A:1067-1069, 2015.

Expression of the target negative regulator gene can be inhibited or, in some embodiments, inactivated, such that the gene does not express an active protein product. In some embodiments, a population of cells can be enriched for cells in which the gene is inactivated.

In some embodiments, the T-cell negative regulator gene that is modified to inhibit expression is A ALAS1, AMBRA1, ANKRD32, ARHGAPI5, Cl5orf40, C3orf33, C8orf44, CARKD, CD300LB, CENPB, CHL1, CHST3, CLEC4M, COLI5A1, COL25A1, COROJA, CUL3, CWC27, CYC1, DOK2, DUSP4, ENG, FAM49B, FKBP1A, FUBP1, GAB3, GLRX GREB1L, GTF2H2, GTF2I, HAUS1, HISTIH2AD, HIST1H2BC, HOXA10, IGFBP4, IRF2BP2, IYD, KCNK4, KDM6B, LICAM, LAMA3, LHX8, MOB3C, MBTD1, MRPL17, MRPL33, MTIF2, MYO1H, NEFL, NFκB1A, NFκB2, NMT1, ORC6, PCBP2, PCGFJ, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNF185, RNF7, RPRD1B, RPS6KL1, SELIL3, SEPW1, SH3BGRL, SIRT7, SIT1, SLC47A1, SLC9A3, SP1, ST5, STAT6, TBL1Y, TGFBR1, TGFBR2, TGIF2, TICRR, TMEM62, TNK1, TNS2, TP53BP1, TTN, TUFM, UPK1B, UQCRC1, WWOX XCL1, ZBTB7A, ZFYVE28, ZNF101, ZNF436, ZNF506, ZNF716, and ZNF805. In some embodiments, a hematopoietic cell, e.g., a T cell such as an effector T cell, further comprises a second modification to inhibit expression of a second T-cell negative regulator gene.

Any number of assays can be used to assess function. Illustrative assays measure T-cell proliferative responses, e.g., in response to T cell receptor (TCR) stimulation. Exemplary assays are described in the EXAMPLES section. Assay include, but are not limited to, CFSE (or other similar dye) dilution, growth-based assays, in vivo expansion at a particular site, or sorting for the other markers of activation or effector function, e.g. cytokine production, induction of a cell surface marker, or granzyme production.

In some embodiments, the T-cell negative regulator gene is inactivated by a gene deletion. As used herein, “gene deletion” refers to removal of at least a portion of a DNA sequence from, or in proximity to, a gene. In some embodiments, the sequence subjected to gene deletion comprises an exonic sequence of a gene. In some embodiments, the sequence subjected to gene deletion comprises a promoter sequence of the gene. In some embodiments, the sequence subjected to gene deletion comprises a flanking sequence of a gene. In some embodiments, a portion of a gene sequence is removed from a gene. In some embodiments, the complete gene sequence is removed from a chromosome. In some embodiments, the host cell comprises a gene deletion as described in the any of the embodiments herein. In some embodiments, the gene is inactivated by deletion of at least one nucleotide or nucleotide base pair in a gene sequence results in a non-functional gene product. In some embodiments, the gene is inactivated by a gene deletion, wherein deletion of at least one nucleotide to a gene sequence results in a gene product that no longer has the original gene product function or activity; or is a dysfunctional gene product. In some embodiments, the gene is inactivated by a gene addition or substitution, wherein addition or substitution of at least one nucleotide or nucleotide base pair into the gene sequence results in a non-functional gene product. In some embodiments, the gene is inactivated by a gene inactivation, wherein incorporation or substitution of at least one nucleotide to the gene sequence results in a gene product that no longer has the original gene product function or activity; or is a dysfunctional gene product. In some embodiments, the gene is inactivated by an addition or substitution, wherein incorporation or substitution of at least one nucleotide into the gene sequence results in a dysfunctional gene product. In some embodiments, the host cell comprises a gene deletion as described in the any of the embodiments herein.

Methods and techniques for inactivating a T-cell negative regulator gene in a host cell, or inactivating a target gene as described herein to suppress T cell function, include, but are not limited to, small interfering RNA (siRNA), small hairpin RNA (shRNA; also referred to as a short hairpin RNA), clustered, regularly interspaced, short palindromic repeats (CRISPR), transcription activator-like effector nuclease (TALEN), zinc-finger nuclease (ZFN), homologous recombination, non-homologous end-joining, and meganuclease. See, e.g., O'Keefe, Mater Methods, 3, 2013; Doench et al., Nat Biotechnol, 32, 2014; Gaj et al., Trends Biotechnol, 31, 2014; and Silva et al., Curr Gene Ther, 11, 2011.

### Inhibitory RNA

In some embodiments, the T-cell negative regulator geneis inactivated by a small interfering RNA (siRNA) system. siRNA sequences to inactivate a target gene can be identified using considerations such as length of siRNA, e.g., 21-23 nucleotides, or fewer; avoidance of regions with 50-100 nucleotides of the start codon and termination codon, avoidance of intron regions; avoidance of stretches of four or more of the same nucleotide; avoidance of regions with GC content that is less than 30% or greater than 60%; avoidance of repeats and low sequence complexity region; avoidance of single nucleotide polymorphic sites, and avoidance of sequences that are complementary to sequences in other off-target genes (see, e.g., Rules of siRNA design for RNA interference, Protocol Online, May 29, 2004; and Reynolds et al., Nat Biotechnol, 22:3236-330 2004).

In some embodiments, the siRNA system comprises a siRNA nucleotide sequence that is about 10 to 200 nucleotides in length, or about 10 to 100 nucleotides in length, or about 15 to 100 nucleotides in length, or about 10 to 60 nucleotides in length, or about 15 to 60 nucleotides in length, or about 10 to 50 nucleotides in length, or about 15 to 50 nucleotides in length, or about 10 to 30 nucleotides in length, or about 15 to 30 nucleotides in length. In some embodiments, the siRNA nucleotide sequence is approximately 10-25 nucleotides in length. In some embodiments, the siRNA nucleotide sequence is approximately 15-25 nucleotides in length. In some embodiments, the siRNA nucleotide sequence is at least about 10, at least about 15, at least about 20, or at least about 25 nucleotides in length. In some embodiments, the siRNA system comprises a nucleotide sequence that is at least about 80%, at least about 85%, at least about 90%, at least about 95%, or 100% complementary to a region of the target mRNA molecule. In some embodiments, the siRNA system comprises a nucleotide sequence that is at least at least about 80%, at least about 85%, at least about 90%, at least about 95%, or 100% complementary to a region of the target pro-mRNA molecule. In some, embodiments, the siRNA system comprises a double stranded RNA molecule. In some embodiments, the siRNA system comprises a single stranded RNA molecule. In some embodiments, the host cell comprises a siRNA system as described in the any of the embodiments herein. In some embodiments, the host cell comprises a pro-siRNA nucleotide sequence that is processed into an active siRNA molecule as described in the any of the embodiments herein. In some embodiments, the host cell comprises a siRNA nucleotide sequence that is at least about 80%, at least about 85%, at least about 90%, at least about 95%, or 100% complementary to a region of the target mRNA molecule. In some embodiments, the host cell comprises an expression vector encoding a siRNA molecule as described in the any of the embodiments herein. In some embodiments, the host cell comprises an expression vector encoding a pro-siRNA molecule as described in the any of the embodiments herein.

In some embodiments, the siRNA system comprises a delivery vector. In some embodiments, the host cell comprises a delivery vector. In some embodiments, the delivery vector comprises the pro-siRNA and/or siRNA molecule.

In some embodiments, the T-cell inegative regulator gene is inactivated by a small hairpin RNA (shRNA; also referred to as a short hairpin RNA) system. Gene inactivation by shRNA systems are available. In some embodiments, the shRNA system comprises a nucleotide sequence that is about 10 to 200 nucleotides in length, or about 10 to 100 nucleotides in length, or about 15 to 100 nucleotides in length, or about 10 to 60 nucleotides in length, or about 15 to 60 nucleotides in length, or about 10 to 50 nucleotides in length, or about 15 to 50 nucleotides in length, or about 10 to 30 nucleotides in length, or about 15 to 30 nucleotides in length. In some embodiments, the shRNA nucleotide sequence is approximately 10-25 nucleotides in length. In some embodiments, the shRNA nucleotide sequence is approximately 15-25 nucleotides in length. In some embodiments, the shRNA nucleotide sequence is at least about 10, at least about 15, at least about 20, or at least about nucleotides in length. In some embodiments, the shRNA system comprises a nucleotide sequence that is at least about 80%, at least about 85%, at least about 90%, at least about 95%, or 100% complementary to a region of a T-cell inhibitory nucleic acid mRNA molecule. In some embodiments, the shRNA system comprises a nucleotide sequence that is at least about 80%, at least about 85%, at least about 90%, at least about 95%, or 100% complementary to a region of a pro-mRNA molecule. In some, embodiments, the shRNA system comprises a double stranded RNA molecule. In some embodiments, the shRNA system comprises a single stranded RNA molecule. In some embodiments, the host cell comprises a shRNA system as described in the any of the embodiments herein. In some embodiments, the host cell comprises a pre-shRNA nucleotide sequence that is processed in an active shRNA nucleotide sequence as described in any of the embodiments herein. In some embodiments, the pro-shRNA molecule composed of DNA. In some embodiments, the pro-shRNA molecule is a DNA construct. In some embodiments, the host cell comprises a shRNA nucleotide sequence that is at least about 80%, at least about 85%, at least about 90%, at least about 95%, or 100% complementary to a region of the T-cell negative regulator gene mRNA molecule. In some embodiments, the host cell comprises an expression vector encoding a shRNA molecule as described in the any of the embodiments herein. In some embodiments, the host cell comprises an expression vector encoding a pro-shRNA molecule as described in the any of the embodiments herein.

In some embodiments, the shRNA system comprises a delivery vector. In some embodiments, the host comprises a delivery vector. In some embodiments, the delivery vector comprises the pro-shRNA and/or shRNA molecule. In some embodiments, the delivery vector is a virus vector. In some embodiments, the delivery vector is a lentivirus. In some embodiments, the delivery vector is an adenovirus. In some embodiments, the vector comprises a promoter.

### CRISPR

In some embodiments, inhibiting expression of a T cell negative regulator gene is accomplished using CRISPR/CAS methodology. Illustrative methods of using the CRISPR/Cas system to reduce gene expression are described in various publications, e.g., U.S. Patent Application Publication No. 2014/0170753. A CRISPR/Cas system includes a Cas protein and at least one to two ribonucleic acids that hybridize to a target motif in the T cell negative regulator gene and direct the Cas protein to the target motif. Any CRISPR/Cas system that is capable of altering a target polynucleotide sequence in a cell can be used. In some embodiments, the CRISPR Cas system is a CRISPR type I system, in some embodiments, the CRISPR/Cas system is a CRISPR type II system. In some embodiments, the CRISPR/Cas system is a CRISPR type V system.

The Cas protein used in the invention can be a naturally occurring Cas protein or a functional derivative thereof. A “functional derivative” includes, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof such as derivative Cas proteins. Suitable derivatives of a Cas polypeptide or a fragment thereof include but are not limited to mutants, fusions, covalent modifications of Cas protein or a fragment thereof.

There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, and Cas9. These Cas nucleases are known to those skilled in the art. For example, the amino acid sequence of the Streptococcus pyogenes wild-type Cas9 polypeptide is set forth, e.g., in NBCI Ref. Seq. No. NP 269215, and the amino acid sequence of Streptococcus thermophilus wild-type Cas9 polypeptide is set forth, e.g., in NBCI Ref. Seq. No. WP_011681470. Some CRISPR-related endonucleases that may be used in methods described herein are disclosed, e.g., in U.S. Application Publication Nos. 2014/0068797, 2014/0302563, and 2014/0356959. Non-limiting examples of Cas nucleases include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologs thereof, variants thereof, mutants thereof, and derivatives thereof.

Cas9 homologs are found in a wide variety of eubacteria, including, but not limited to bacteria of the following taxonomic groups: Actinobacteria, Aquificae, Bacteroidetes-Chlorobi, Chlamydiae-Verrucomicrobia, Chlroflexi, Cyanobacteria, Firmicutes, Proteobacteria, Spirochaetes, and Thermotogae. An exemplary Cas9 protein is the Streptococcus pyogenes Cas9 protein. Additional Cas9 proteins and homologs thereof are described in, e.g., Chylinksi, et al., RNA Biol. 2013-5-1; 10(5): 726-737; Nat. Rev. Microbiol. 2011 June; 9(6): 467-477; Hou, et al., Proc Natl Acad Sci USA. 2013 Sep. 24; 110(39):15644-9; Sampson et al., Nature. 2013-5-9; 497(7448):254-7; and Jinek, et al., Science. 2012-8-17; 337(6096):816-21. Variants of any of the Cas9 nucleases provided herein can be optimized for efficient activity or enhanced stability in the host cell. Thus, engineered Cas9 nucleases are also contemplated. Cas 9 from Streptococcus pyogenes contains 2 endonuclease domains, including an RuvC-like domain that cleaves target DNA that is noncomplementary to crRNA, and an HNH nuclease domain that cleave target DNA complementary to crRNA. The double-stranded endonuclease activity of Cas9 also involves a short conserved sequence, (2-5 nucleotides), known as a protospacer-associated motif (PAM), which follows immediately 3′- of a target motif in the target sequence

Additionally, Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, Streptococcus mutans, Listeria innocua, Staphylococcus pseudintermedius, Acidaminococcus intestine, Olsenella uli, Oenococcus kitaharae, Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, lyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae, Azospirillum, Nitrobacter hamburgensis, Bradyrhizobium, Wolinella succinogenes, Campylobacter jejuni subsp. Jejuni, Helicobacter mustelae, Bacillus cereus, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

Other RNA-mediated nucleases include Cpf1 (See, e.g., Zetsche et al., Cell, Volume 163, Issue 3, p759-771, 22-10-2015) and homologs thereof.

As used herein, the term “Cas9 ribonucleoprotein” complex and the like refers to a complex between the Cas9 protein and a guide RNA, the Cas9 protein and a crRNA, the Cas9 protein and a trans-activating crRNA (tracrRNA), or a combination thereof (e.g., a complex containing the Cas9 protein, a tracrRNA, and a crRNA guide RNA). It is understood that in any of the embodiments described herein, a Cas9 nuclease can be subsitututed with another RNA-mediated nuclease, e.g., an alternative Cas protein or a Cpf1 nuclease.

In some embodiments, the Cas protein is introduced into T-cells in polypeptide form. Thus, for example, in certain embodiments, the Cas proteins can be conjugated to or fused to a cell-penetrating polypeptide or cell-penetrating peptide that is well known in the art. Non-limiting examples of cell-penetrating peptides include those provided in Milletti F, “Drug Discov. Today 17: 850-860, 2012, the relevant disclosure of which is hereby incorporated by reference in its entirety. In some cases, T cells may be genetically engineered to produce the Cas protein.

In some embodiments, a Cpf1 nuclease or the Cas9 nuclease and the gRNA are introduced into the cell as a ribonucleoprotein (RNP) complex.

In some embodiments, the RNP complex may be introduced into about 1×10⁵ to about 2×10⁶ cells (e.g., 1×10⁵ cells to about 5×10⁵ cells, about 1×10⁵ cells to about 1×10⁶ cells, 1×10⁵ cells to about 1.5×10⁶ cells, 1×10⁵ cells to about 2×10⁶ cells, about 1×10⁶ cells to about 1.5×10⁶ cells, or about 1×10⁶ cells to about 2×10⁶ cells). In some embodiments, the cells are cultured under conditions effective for expanding the population of modified cells. Also disclosed herein is a population of cells, in which the genome of at least 20%, 30%, 40%, 50%, 60%, 70%, 80%, 90%, 95%, 99% or greater of the cells comprises a genetic modification or heterologous polynucleotide that inhibits expression of a T cell negative regulator gene s as described herein. In some embodiments, the population comprises subpopulations of cells each of which subpopulations have a different genetic modification to inhibit expression of a T cell negative regulator gene as described herein.

In some embodiments, the RNP complex is introduced into the T cells by electroporation. Methods, compositions, and devices for electroporating cells to introduce a RNP complex are available in the art, see, e.g., WO 2016/123578, WO/2006/001614, and Kim, J. A. et al. Biosens. Bioelectron. 23, 1353-1360 (2008). Additional or alternative methods, compositions, and devices for electroporating cells to introduce a RNP complex can include those described in U.S. Patent Appl. Pub. Nos. 2006/0094095; 2005/0064596; or 2006/0087522; Li, L. H. et al. Cancer Res. Treat. 1, 341-350 (2002); U.S. Pat. Nos. 6,773,669; 7,186,559; 7,771,984; 7,991,559; 6,485,961; 7,029,916; and U.S. Patent Appl. Pub. Nos: 2014/0017213; and 2012/0088842; Geng, T. et al., J. Control Release 144, 91-100 (2010); and Wang, J., et al. Lab. Chip 10, 2057-2061 (2010).

In some embodiments, the Cas9 protein can be in an active endonuclease form, such that when bound to target nucleic acid as part of a complex with a guide RNA or part of a complex with a DNA template, a double strand break is introduced into the target nucleic acid. In the methods provided herein, a Cas9 polypeptide or a nucleic acid encoding a Cas9 polypeptide can be introduced into the T cell. The double strand break can be repaired by HDR to insert the DNA template into the genome of the T cell. Various Cas9 nucleases can be utilized in the methods described herein. For example, a Cas9 nuclease that requires an NGG protospacer adjacent motif (PAM) immediately 3′ of the region targeted by the guide RNA can be utilized. Such Cas9 nucleases can be targeted to a region in exon 1 of the TRAC or exon 1 of the TRAB that contains an NGG sequence. As another example, Cas9 proteins with orthogonal PAM motif requirements can be used to target sequences that do not have an adjacent NGG PAM sequence. Exemplary Cas9 proteins with orthogonal PAM sequence specificities include, but are not limited to those described in Esvelt et al., Nature Methods 10: 1116-1121 (2013).

In some cases, the Cas9 protein is a nickase, such that when bound to target nucleic acid as part of a complex with a guide RNA, a single strand break or nick is introduced into the target nucleic acid. A pair of Cas9 nickases, each bound to a structurally different guide RNA, can be targeted to two proximal sites of a target genomic region and thus introduce a pair of proximal single stranded breaks into the target genomic region, for example exon 1 of a TRAC gene or exon 1 of a TRBC gene. Nickase pairs can provide enhanced specificity because off-target effects are likely to result in single nicks, which are generally repaired without lesion by base-excision repair mechanisms. Illustrative Cas9 nickases include Cas9 nucleases having a D10A or H840A mutation (See, for example, Jinek et al., Science 337:816-821, 2012; Qi et al., Cell, 152(5):1173-1183, 2012; Ran et al., Cell 154: 1380-1389, 2013). In one embodiment, the Cas9 polypeptide from Streptococcus pyogenes comprises at least one mutation at position D10, G12, G17, E762, H840, N854, N863, H982, H983, A984, D986, A987 or any combination thereof. Descriptions of such dCas9 polypeptides and variants thereof are provided in, for example, International Patent Publication No. WO 2013/176772. The Cas9 enzyme may contain a mutation at D10, E762, H983, or D986, as well as a mutation at H840 or N863. In some instances, the Cas9 enzyme may contain a D10A or DION mutation. In further embodiments, the Cas9 enzyme may contain a H840A, H840Y, or H840N. In some embodiments, the Cas9 enzyme may contain D10A and H840A; D10A and H840Y; D10A and H840N; D10N and H840A; D10N and H840Y; or D10N and H840N substitutions. The substitutions can be conservative or non-conservative substitutions to render the Cas9 polypeptide catalytically inactive and able to bind to target DNA.

In some embodiments, the Cas nuclease can be a high-fidelity or enhanced specificity Cas9 polypeptide variant with reduced off-target effects and robust on-target cleavage. Non-limiting examples of Cas9 polypeptide variants with improved on-target specificity include the SpCas9 (K855A), SpCas9 (K810A/K1003A/R1060A) (also referred to as eSpCas9(1.0)), and SpCas9 (K848A/K1003A/R1060A) (also referred to as eSpCas9(1.1)) variants described in Slaymaker et al., Science, 351(6268):84-8 (2016), and the SpCas9 variants described in Kleinstiver et al., Nature, 529(7587):490-5 (2016) containing one, two, three, or four of the following mutations: N497A, R661A, Q695A, and Q926A (e.g., SpCas9-HF1 contains all four mutations).

In some embodiments, the target motifs can be selected to minimize off-target effects of the CRISPR/Cas systems of the present invention. For example, in some embodiments, the target motif is selected such that it contains at least two mismatches when compared with all other genomic nucleotide sequences in the cell. In some embodiments, the target motif is selected such that it contains at least one mismatch when compared with all other genomic nucleotide sequences in the cell. Those skilled in the art will appreciate that a variety of techniques can be used to select suitable target motifs for minimizing off-target effects (e.g., bioinformatics analyses).

In some embodiments, CRISPRi is employed for sequence-specific repression of gene expression of a T-cell negative regulator gene described herein. Description of CRISPRi methods is provided, e.g., in Engreitz et al., Cold Spring Harb Perspect Biol, 2019, 1 l:a035386. In some embodiments, the CRISPRi system includes a dCas9 polypeptide or a dCasl2 polypeptide operably linked to a repression domain. In some embodiments, the repression domain is selected from the group consisting of a Kriippel-associated box (KRAB) repressor domain, a NuE repressor domain, a NcoR repressor domain, a SID repressor domain, a SID4X repressor domain, an EZH2 repressor domain, a FOG repressor domain, a DNMT3 A repressor domain, and a DNMT3L repressor domain.

In some embodiments, CRISPRoff is employed to silence a T-cell negative regulator gene (see, e.g., Nuñez J K, Chen J, Pommier G C, et al. Genome-wide programmable transcriptional memory by CRISPR-based epigenome editing. Cell, 2021; 0(0). doi: 10.1016/j.cell.2021.03.02.)

In some embodiments, base editing to introduce point mutations into a T-cell negative regulator gene. DNA base editors comprise fusions between a catalytically impaired Cas nuclease and a base-modification enzyme that operates on single-stranded DNA (ssDNA) but not double-stranded DNA (dsDNA). Upon binding to its target locus in DNA, base pairing between a guide RNA and target DNA strand leads to displacement of a small segment of single-stranded DNA in an R loop. DNA bases within this single-stranded DNA bubble are modified by the deaminase enzyme. To improve efficiency in eukaryotic cells, the catalytically disabled nuclease also generates a nick in the non-edited DNA strand, inducing cells to repair the non-edited strand using the edited strand as a template. DNA base editors are available that can mediate all four possible transition mutations (C to T, A to G, T to C, and G to A). See, for example Rees & Liu, Nat. Rev. Genet. 19:770-788, 2008 and references cited therein.

As used throughout, a guide RNA (gRNA) sequence is a sequence that interacts with a site-specific or targeted nuclease and specifically binds to or hybridizes to a target nucleic acid within the genome of a cell, such that the gRNA and the targeted nuclease co-localize to the target nucleic acid in the genome of the cell. Each gRNA includes a DNA targeting sequence or protospacer sequence of about 10 to 50 nucleotides in length that specifically binds to or hybridizes to a target DNA sequence in the genome. For example, the targeting sequence may be about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some embodiments, the gRNA comprises a crRNA sequence and a transactivating crRNA (tracrRNA) sequence. In some embodiments, the gRNA does not comprise a tracrRNA sequence.

The sgRNAs can be selected depending on the particular CRISPR/Cas system employed, and the sequence of the target polynucleotide, as will be appreciated by those skilled in the art. As indicated above, in some embodiments, the one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs. Guide RNAs can also be designed using software that are readily available, for example, at the website crispr.mit.edu. The one or more sgRNAs can be transfected into T cells in which Cas protein is present by transfection, according to methods known in the art.

In some cases, the DNA targeting sequence can incorporate wobble or degenerate bases to bind multiple genetic elements. In some cases, the 19 nucleotides at the 3′ or 5′ end of the binding region are perfectly complementary to the target genetic element or elements. In some cases, the binding region can be altered to increase stability. For example, non-natural nucleotides, can be incorporated to increase RNA resistance to degradation. In some cases, the binding region can be altered or designed to avoid or reduce secondary structure formation in the binding region. In some cases, the binding region can be designed to optimize G-C content. In some cases, G-C content is preferably between about 40% and about 60% (e.g., 40%, 45%, 50%, 55%, 60%).

In some embodiments, the sequence of the gRNA or a portion thereof is designed to complement (e.g., perfectly complement) or substantially complement (e.g., 50%, 55%, 60%, 65%, 70%, 75%, 80%, 85%, 90%, 91%, 92%, 93%, 94% 95%, 96%, 97%, 98%, or 99% complement) the target region in the T-cell negative regulator gene. In some embodiments, the portion of the gRNA that complements and binds the targeting region in the polynucleotide is, or is about, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 or more nucleotides in length. In some cases, the portion of the gRNA that complements and binds the targeting region in the polynucleotide is between about 19 and about 21 nucleotides in length. In some cases, the gRNA may incorporate wobble or degenerate bases to bind target regions. In some cases, the gRNA can be altered to increase stability. For example, non-natural nucleotides, can be incorporated to increase RNA resistance to degradation. In some cases, the gRNA can be altered or designed to avoid or reduce secondary structure formation. In some cases, the gRNA can be designed to optimize G-C content. In some cases, G-C content is between about 40% and about 60% (e.g., 40%, 45%, 50%, 55%, 60%). In some cases, the binding region can contain modified nucleotides such as, without limitation, methylated or phosphorylated nucleotides.

In some embodiments, the gRNA can be optimized for expression by substituting, deleting, or adding one or more nucleotides. In some cases, a nucleotide sequence that provides inefficient transcription from an encoding template nucleic acid can be deleted or substituted. For example, in some cases, the gRNA is transcribed from a nucleic acid operably linked to an RNA polymerase III promoter. In such cases, gRNA sequences that result in inefficient transcription by RNA polymerase III, such as those described in Nielsen et al., Science. 2013-6-28; 340(6140):1577-80, can be deleted or substituted. For example, one or more consecutive uracils can be deleted or substituted from the gRNA sequence. In some cases, if the uracil is hydrogen bonded to a corresponding adenine, the gRNA sequence can be altered to exchange the adenine and uracil. This “A-U flip” can retain the overall structure and function of the gRNA molecule while improving expression by reducing the number of consecutive uracil nucleotides.

In some embodiments, the gRNA can be optimized for stability. Stability can be enhanced by optimizing the stability of the gRNA:nuclease interaction, optimizing assembly of the gRNA:nuclease complex, removing or altering RNA destabilizing sequence elements, or adding RNA stabilizing sequence elements. In some embodiments, the gRNA contains a 5′ stem-loop structure proximal to, or adjacent to, the region that interacts with the gRNA-mediated nuclease. Optimization of the 5′ stem-loop structure can provide enhanced stability or assembly of the gRNA:nuclease complex. In some cases, the 5′ stem-loop structure is optimized by increasing the length of the stem portion of the stem-loop structure.

gRNAs can be modified by methods known in the art. In some cases, the modifications can include, but are not limited to, the addition of one or more of the following sequence elements: a 5′ cap (e.g., a 7-methylguanylate cap); a 3′ polyadenylated tail; a riboswitch sequence; a stability control sequence; a hairpin; a subcellular localization sequence; a detection sequence or label; or a binding site for one or more proteins. Modifications can also include the introduction of non-natural nucleotides including, but not limited to, one or more of the following: fluorescent nucleotides and methylated nucleotides.

Also provided herein are expression cassettes and vectors for producing gRNAs in a host cell. The expression cassettes can contain a promoter (e.g., a heterologous promoter) operably linked to a polynucleotide encoding a gRNA. The promoter can be inducible or constitutive. The promoter can be tissue specific. In some cases, the promoter is a U6, H1, or spleen focus-forming virus (SFFV) long terminal repeat promoter. In some cases, the promoter is a weak mammalian promoter as compared to the human elongation factor 1 promoter (EF1A). In some cases, the weak mammalian promoter is a ubiquitin C promoter or a phosphoglycerate kinase 1 promoter (PGK). In some cases, the weak mammalian promoter is a TetOn promoter in the absence of an inducer. In some cases, when a TetOn promoter is utilized, the host cell is also contacted with a tetracycline transactivator. In some embodiments, the strength of the selected gRNA promoter is selected to express an amount of gRNA that is proportional to the amount of Cas9 or dCas9. The expression cassette can be in a vector, such as a plasmid, a viral vector, a lentiviral vector, etc. In some cases, the expression cassette is in a host cell. The gRNA expression cassette can be episomal or integrated in the host cell.

### Modifications Using Alternative Targeted Nuclease Systems

In some embodiments, a targeted nuclease that is employed in modifying a T cell to inhibit expression of a T-cell regulatory gene a transcription activator-like effector nuclease (TALEN), a zinc finger nuclease (ZFN) or a megaTAL (See, for example, Merkert and Martin “Site-Specific Genome Engineering in Human Pluripotent Stem Cells,” Int. J. Mol. Sci. 18(7): 1000 (2016)).

### Zinc-Finger Nuclease to Inhibit T-Cell Negative Regulator Gene Expression

In some embodiments, modified T cells comprising a T-cell negative regulator gene-targeted alteration are produced by inhibiting expression using ZFN. Methods of using the ZFNs to reduce gene expression are described, e.g., in U.S. Pat. No. 9,045,763, and also in Durai et al., Nucleic Acid Research 33:5978-5990, 2005; Carroll et al. Genetics Society of America 188: 773-782, 2011; and Kim et al. Proc. Natl. Acad. Sci. USA 93: 1156-1160.

A ZFN comprises a FokI nuclease domain (or derivative thereof) fused to a DNA-binding domain. In the case of a ZFN, the DNA-binding domain comprises one or more zinc fingers. A zinc finger is a small protein structural motif stabilized by one or more zinc ions. A zinc finger can comprise, for example, Cys2His2, and can recognize an approximately 3-bp sequence. Various zinc fingers of known specificity can be combined to produce multi-finger polypeptides which recognize about 6, 9, 12, 15 or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells.

A ZFN dimerizes to cleave DNA. Thus, a pair of ZFNs are used to target non-palindromic DNA sites. The two individual ZFNs bind opposite strands of the DNA with their nucleases properly spaced apart (see, e.g., Bitinaite et al., Proc. Natl. Acad. Sci. USA 95: 10570-5, 1998). A ZFN can create a double-stranded break in the DNA, which can create a frame-shift mutation if improperly repaired, leading to a decrease in the expression and level of expression of the target gene in a cell in a cell.

### TALENs to Inhibit T-Cell Negative Regulator Genes

In some embodiments, T-cells that comprise a targeted alteration are produced by inhibiting the desired T-cell negative regulator gene with transcription activator-like effector nucleases (TALENS). TALENs are similar to ZFNs in that they bind as a pair around a genomic site and direct a non-specific nuclease, e.g., FoKI, to cleave the genome at a specific site, but instead of recognizing DNA triplets, each domain recognizes a single nucleotide. Methods of using TALENS to reduce gene expression are disclosed, e.g., in U.S. Pat. No. 9,005,973; Christian et al. “Genetics 186(2): 757-761, 2010; Zhang et al. 2011 Nature Biotech. 29: 149-53, 2011; Geibler et al. 2011 PLoS ONE 6: e19509, 2011; Boch et al. 2009 Science 326: 1509-12; Moscou et al. 2009 Science 326: 3501.

To produce a TALEN, a TALE protein is typically fused to a FokI endonuclease, which can be a wild-type or mutated FokI endonuclease. Several mutations to FokI have been made for its use in TALENs; these, for example, improve cleavage specificity or activity. Cermak et al., Nucl. Acids Res. 39:e82, 2011; Miller et al., Nature Biotech. 29:143-8, 2011; Hockemeyer et al., Nature Biotech. 29:731-734, 2011; Wood et al., Science 333:307, 2011; Doyon et al., Nature Methods 8:74-79, 2010; Szczepek et al., Nature Biotech. 25:786-793, 2007; and Guo et al., J Mol. Biol. 200:96, 2010.

The FokI domain functions as a dimer and typically employ two constructs with unique DNA binding domains for sites in the target genome with proper orientation and spacing. Both the number of amino acid residues between the TALE DNA binding domain and the FokI cleavage domain and the number of bases between the two individual TALEN binding sites appear to be important parameters for achieving high levels of activity. (e.g., Miller et al., 2011, supra).

### Meganucleases

“Meganucleases” are rare-cutting endonucleases or homing endonucleases that can be highly specific, recognizing DNA target sites ranging from at least 12 base pairs in length, e.g., from 12 to 40 base pairs or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. The meganuclease can be monomeric or dimeric.

In some embodiments of the methods described herein, meganucleases may be used to inhibit the expression of a T-cell negative regulator gene or inhibit expression of a gene to suppress immune function as described herein. In some instances, the meganuclease is naturally-occurring (found in nature) or wild-type, and in other instances, the meganuclease is non-natural, artificial, engineered, synthetic, or rationally designed. In certain embodiments, the meganucleases that may be used in methods described herein include, but are not limited to, an I-CreI meganuclease, I-CeuI meganuclease, I-MsoI meganuclease, I-SceI meganuclease, variants thereof, mutants thereof, and derivatives thereof.

Detailed descriptions of useful meganucleases and their application in gene editing are found, e.g., in Silva et al., Curr Gene Ther, 2011, 11(1):11-27; Zaslavoskiy et al., BMC Bioinformatics, 2014, 15:191; Takeuchi et al., Proc Nal Acad Sci USA, 2014, 111(11):4061-4066, and U.S. Pat. Nos. 7,842,489; 7,897,372; 8,021,867; 8,163,514; 8,133,697; 8,021,867; 8,119,361; 8,119,381; 8,124,36; and 8,129,134.

Efficiency of the inhibition of expression of any T-cell regulatory gene using a method as described herein can be assessed by measuring the amount of mRNA or protein using methods well known in the art, for example, quantitative PCR, western blot, flow cytometry, etc and the like. In some embodiments, the level of protein is evaluated to assess efficiency of inhibition efficiency. In certain embodiments, the efficiency of reduction of target gene expression is at least 5%, at least 10%, at least 20%, at least 30%, at least 50%, at least 60%, or at least 80%, or at least 90%, or greater. as compared to corresponding cells that do not have the targeted modification. In certain embodiments, the efficiency of reduction is from about 10% to about 90%. In certain embodiments, the efficiency of reduction is from about 30% to about 80%. In certain embodiments, the efficiency of reduction is from about 50% to about 80%. In some embodiments, the efficiency of reduction is greater than or equal to about 80%.

### Treatment Methods and Compositions

Any of the methods described herein may be used to modify T cells, e.g., CD8+ T cells, obtained from a human subject. T-cells modified in accordance with the invention may be used to treat any number of cancers, including solid tumors.

### Methods of Treating Cancer

In some embodiments, T cells are modified to decrease expression of one or more T-cell negative regulator genes as described herein. In some embodiments, a T-cell negative regulator gene that is modified is ALAS1, AMBRA1, ANKRD32, ARHGAP15, C15orf40, C3orf33, C8orf44, CARKD, CD300LB, CENPB, CHL1, CHST3, CLEC4M, COLI5A1, COL25A1, CORO1A, CUL3, CWC27, CYC1, DOK2, DUSP4, ENG, FAM49B, FKBPIA, FUBP1, GAB3, GLRX, GREB1L, GTF2H2, GTF2I, HAUS1, HISTIH2AD, HISTIH2BC, HOXAJO, IGFBP4, IRF2BP2, IYD, KCNK4, KDM6B, LICAM, LAMA3, LHX8, MOB3C, MBTD1, MRPL17, MRPL33, MTIF2, MYO1H, NEFL, NFκB1A, NFκB2, NMT1, ORC6, PCBP2, PCGF1, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNF185, RNF7, RPRD1B, RPS6KL1, SELIL3, SEPW1, SH3BGRL, SIRT7, SIT1, SLC47A1, SLC9A3, SP1, ST5, STAT6, TBL1Y, TGFBR1, TGFBR2, TGIF2, TICRR, TMEM62, TNK1, TNS2, TP53BP1, TTN, TUFM, UPK1B, UQCRC1, WWOX XCL1, ZBTB7A, ZFYVE28, ZNF101, ZNF436, ZNF506, ZNF716, and ZNF805. Thus, in some embodiments, provided herein is a method of treating cancer in a human subject comprising: a) obtaining T cells, e.g., CD8+ T cells, from the subject; b) modifying the T cells using any of the methods provided herein to decrease expression of a T cell negative regulator gene, e.g., a gene disclosed in this paragraph; and c) administering the modified T cells to the subject.

In some embodiments, T cells, e.g., CD8+ T cells, obtained from a subject that has cancer may be expanded ex vivo. The characteristics of the subject's cancer may determine a set of tailored cellular modifications (e.g., selection of one or more negative regulator gene targets), and these modifications may be applied to the T cells using any of the methods described herein. Modified Tcells may then be reintroduced to the subject. This strategy capitalizes on and enhances the function of the subject's natural repertoire of cancer specific T cells, providing a diverse arsenal to eliminate mutagenic cancer cells quickly.

Any cancer can be treated with genetically modified T cells as described herein. In some embodiments, the cancer is a carcinoma or a sarcoma. In some embodiments, the cancer is a hematological cancer. In some embodiments, the cancer is breast cancer, prostate cancer, testicular cancer, renal cell cancer, bladder cancer, liver cancer, ovarian cancer, cervical cancer, endometrial cancer, lung cancer, colorectal cancer, anal cancer, pancreatic cancer, gastric cancer, esophageal cancer, hepatocellular cancer, kidney cancer, head and neck cancer, glioblastoma, mesothelioma, melanoma, a chondrosarcoma, or a bone or soft tissue sarcoma. In some embodiments, the cancer is adrenocortical carcinoma, anal cancer, appendix cancer, astrocytoma, basal-cell carcinoma, bile duct cancer, bone tumor, brainstem glioma, brain cancer, cerebellar astrocytoma, cerebral astrocytoma, ependymoma, medulloblastoma, supratentorial primitive neuroectodermal tumors, visual pathway and hypothalamic glioma, or bronchial adenomas. In some embodiments, the cancer is acute lymphoblastic leukemia, acute myeloid leukemia, Burkitt's lymphoma, central nervous system lymphoma, chronic lymphocytic leukemia, chronic myelogenous leukemia, hairy cell leukemia, chronic myeloproliferative disorders, a myelodysplastic syndrome, an adult acute myeloproliferative disorder, multiple myeloma, cutaneous T-cell lymphoma, Hodgkin lymphoma, or non-Hodgkin lymphoma. In some embodiments, the cancer is desmoplastic small round cell tumor, ependymoma, epitheliod hemangioendothelioma (EHE), Ewing's sarcoma, extracranial germ cell tumor, extragonadal germ cell tumor, extrahepatic bile duct cancer, intraocular melanoma, retinoblastoma, gallbladder cancer, gastrointestinal carcinoid tumor, gastrointestinal stromal tumor (GIST), germ cell tumor, gestational trophoblastic tumor, gastric carcinoid, heart cancer, hypopharyngeal cancer, hypothalamic and visual pathway glioma, childhood, intraocular melanoma, islet cell carcinoma, Kaposi sarcoma, laryngeal cancer, lip and oral cavity cancer, liposarcoma, non-small cell lung cancer, small-cell lung cancer, macroglobulinemia, male breast cancer, malignant fibrous histiocytoma of bone, medulloblastoma, melanoma, Merkel cell cancer, mesothelioma, metastatic squamous neck cancer, mouth cancer, multiple endocrine neoplasia syndrome, mycosis fungoides, chronic, myxoma, nasal cavity and paranasal sinus cancer, nasopharyngeal carcinoma, neuroblastoma, oligodendroglioma, oral cancer, oropharyngeal cancer, osteosarcoma, ovarian epithelial cancer, ovarian germ cell tumor, ovarian low malignant potential tumor, paranasal sinus and nasal cavity cancer, parathyroid cancer, penile cancer, pharyngeal cancer, pheochromocytoma, pineal astrocytoma, pineal germinoma, pineoblastoma, supratentorial primitive neuroectodermal tumors, pituitary adenoma. plasma cell neoplasia, pleuropulmonary blastoma, primary central nervous system lymphoma, renal cell carcinoma, retinoblastoma, rhabdomyosarcoma, salivary gland cancer, uterine sarcoma, Sezary syndrome, non-melanoma skin cancer, melanoma Merkel cell skin carcinoma, small intestine cancer, squamous cell carcinoma, squamous neck cancer, throat cancer, thymoma, thyroid cancer, transitional cell cancer of the renal pelvis and ureter, trophoblastic tumor, gestational, urethral cancer, uterine cancer, vaginal cancer, vulvar cancer, Waldenstrom macroglobulinemia, or Wilms tumor.

In certain embodiments, the genetically modified T cells, or individual populations of sub-types of the genetically modified T cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 104 and at or about 10⁹ cells/kilograms (kg) body weight, such as between 10⁵ and 10⁶ cells/kg body weight, for example, at least about 1×10⁵ cells/kg, 1.5×10⁵ cells/kg, 2×10⁵ cells/kg, 5×10⁵ cells/kg, or 1×10⁶ cells/kg body weight.

The appropriate dosage may depend on the type of cancer to be treated, the severity and course of the disease, previous therapy, the subject's clinical history and response to the cells, and the discretion of the attending physician. The compositions and cells are in some embodiments suitably administered to the subject at one time or over a series of treatments.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents.

Publications cited herein and the material for which they are cited are hereby specifically incorporated by reference in their entireties.

### EXAMPLES

The following examples are offered to illustrate, but not to limit the claimed invention.

### Example 1. Identification of Genes that Play a Role in Immunosuppression in Tumor Microenvironments

The suppressive tumor microenvironment and T cell intrinsic checkpoints can impinge on the efficacy of engineered T cells targeting solid tumors (Lim, et al., Cell 168, 724-740, 2017). We developed a systematic approach to discover genetic perturbations that could render T cells resistant to a range of inhibitory signals encountered in the TME. We previously used an adenosine agonist (Shifrut et al., 2018, supra; WO2020/014235) (CGS-21680) to simulate elevated adenosine A2A inhibitory signaling in response to high levels of adenosine in the hypoxic TME (Sitkovsky, et al., Annu. Rev. Immunol. 22, 657-682, 2004). In this example, this strategy was also extended to model multiple challenges to T cell function in tumor microenvironments. To model intrinsic checkpoint signals, we focused on inhibitors of calcium/calcineurin signaling (tacrolimus and cyclosporine) which is a critical pathway for T cell activation often suppressed in tumor infiltrating T cells (Park, et al, 2020; Martinez, et al., 2015, both supra). To mimic a prominent extrinsic inhibitory signal in the TME, we used TGFβ, a canonical suppressive cytokine limiting T cell function within tumors (Kloss et al, 2018, supra). Lastly, as regulatory T cells (Tregs) are important mediators of T cell dysfunction in multiple tumor types (Plitas et al, 2016, supra), we adapted our screening platform to assay cell-cell interactions and reveal genes that confer resistance to suppression of effector T cells by Tregs.

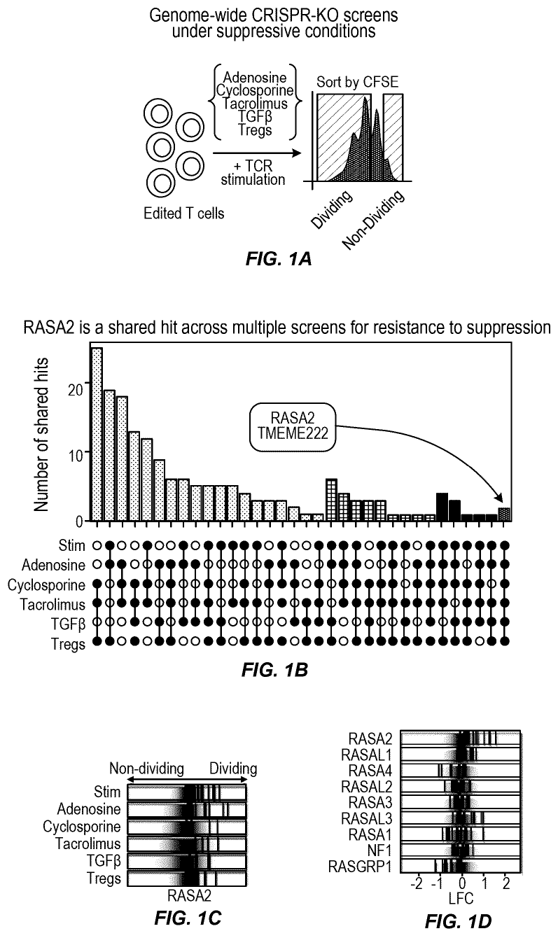

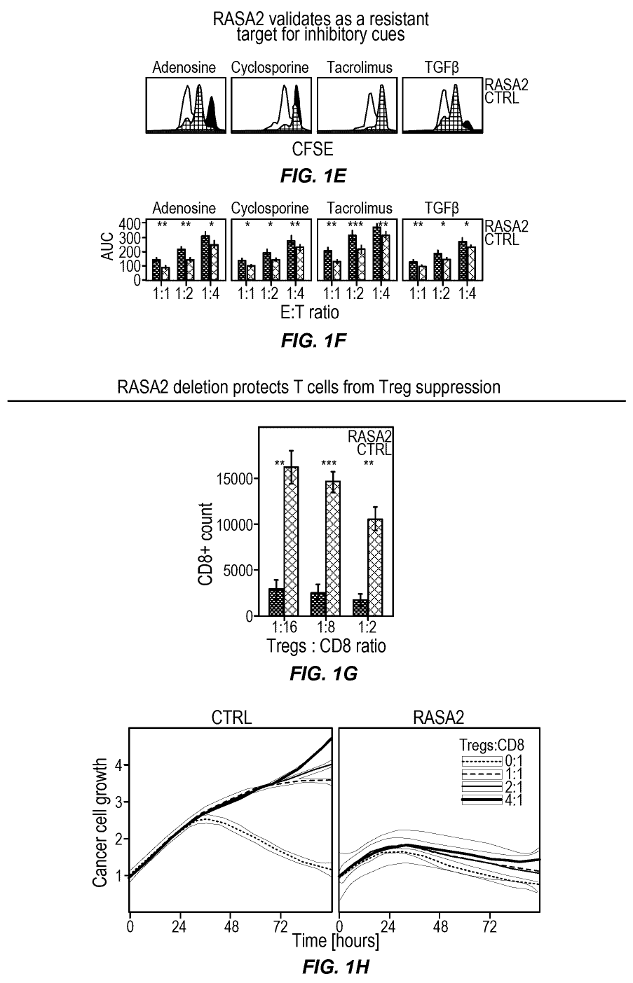

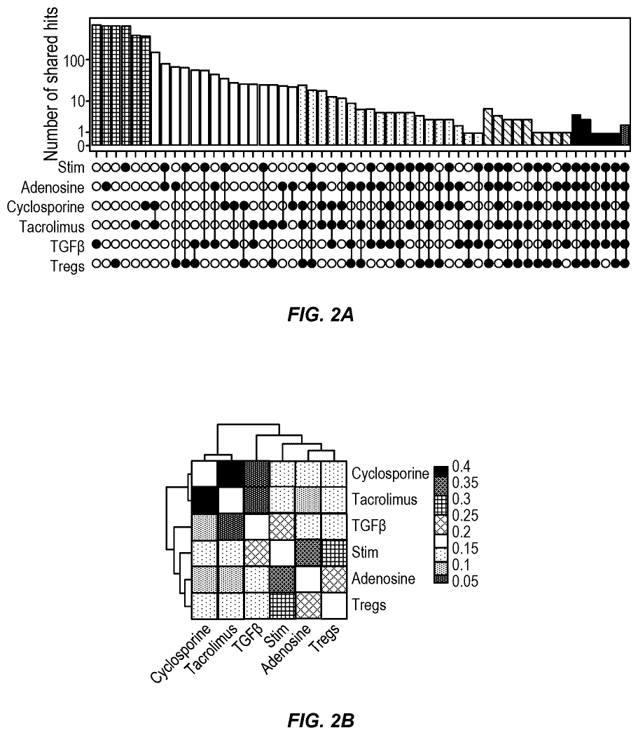

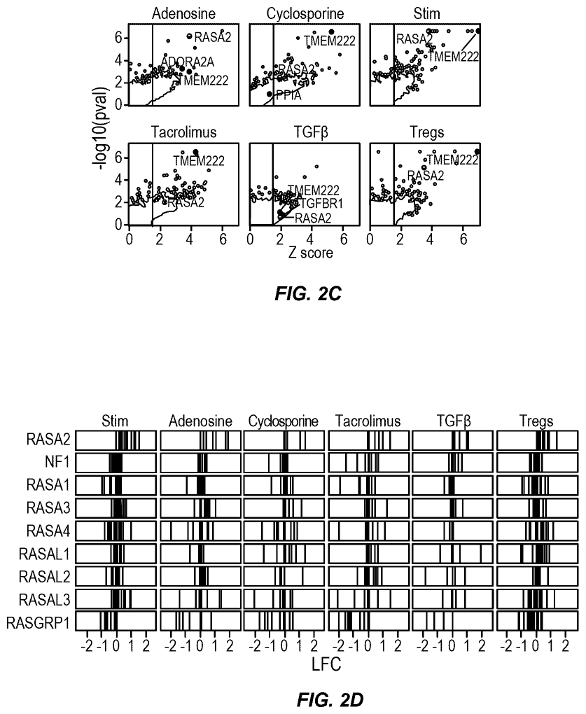

To identify regulators of resistance to these suppressive conditions, we applied our SLICE (sgRNA lentiviral infection with Cas9 electroporation) methodology of pooled genome-wide CRISPR-knockout screens in primary human T cells (Shifrut et al, 2018, WO2020/014235, which is incorporated by reference. We analyzed a total of 6 different genome-wide screens in primary human T cells across multiple independent donors and suppressive conditions (FIG. 1a and methods). In each condition, gene targets that promoted T cell proliferation were identified by FACS sorting to find sgRNAs enriched in the dividing (CFSE low) over those in non-dividing cells (CFSE high) after the cells were restimulated. Analysis of screen hits highlighted the relationship between similar suppressive cues (e.g. tacrolimus and cyclosporine) and confirmed the specificity of these suppression screens with ADORA2A and TGFBR1 scoring highly in the adenosine and TGFβ conditions, respectively (FIG. 2a-c).

These screens identified the following genes under the suppressive conditions as follows:

Adenosine treatment (associated with hypoxic TME): CHST3, TTN, NMT1, RPS6KL1, STAT6, C8orf44, PDCL, TP53BP1, WWOX GLRX, ZNF506, TNS2, and TBL1Y.

### Stim: UQCRC1, IRF2BP2, RPRD1B, AMBRA1, DUSP4, and PCBP2.

Treg suppression screen: CUL3, CORO1A, RFPL1, HISTIH2AD, PLGLB2, SH3BGRL, GLRX, ARHGAP15, CHL1, SIT1, CYC1, AMBRA1, GAB3, DOK2, FUBP1, and PDCD6IP.
Cyclosporine treatement: KDM6B, COL15A1, ZFYVE28, CARKD, ZNF101, HOXA10, C3orf33, ALAS1, CYC1, ZBTB7A, FAM49B, MRPL17, GREB1L, PPP2R5D, SLC9A3, CWC27, and GTF2H2.
Tacrolimus treatment: ZNF716, XCL1, NFκB2, POTEJ, SP1, NEFL, KCNK4, TNK1, CLEC4M, PCGFJ, RNF13, SLC47A1, ZNF436, WWOX ANKRD32, SELIL3, SEPW1, and COL25AL.
TGFβ treatment: T CENPB, CD300LB, IYD, ST5, RNF7, MBTDJ, MRPL33, MYO1H, PIWIL4, ZNF805, HISTIH2BC, UPKIB, LAMA3, ENG, ORC6, TICRR, C15orf40, TUFM, RNF185, PTPRG, HAUS1, TMEM62, IGFBP4, LICAM, and MTIF2.
Additional Analysis and Characterization/Validation of Candidates from the Screen

Further data analysis and characterization/validation experiments were performed for gene targets. In particular, in order to improve the detection of gene targets that are selective or more general to diverse suppressive conditions, we generated new analysis of our screen data by comparing sgRNA enrichment in the highly dividing cells across the different conditions. Using this analysis, we selected gene candidates predicted to confer selective versus more general resistance to the different suppressive conditions for further functional validation in large arrayed experiments.

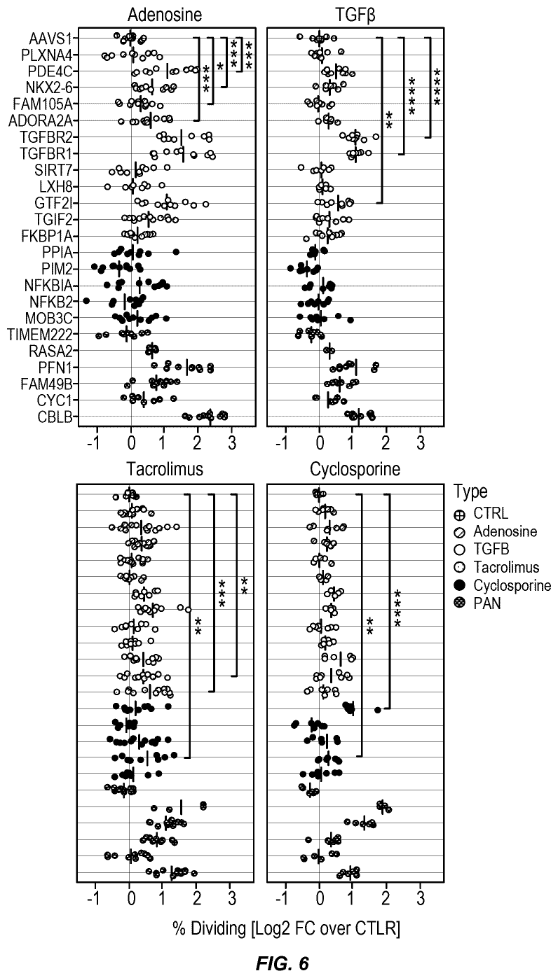

We selected a number of sgRNAs for these targets, including 22 target genes with two sgRNAs per gene (Table 2) and all experiments performed in two human T cell donors. After using CRISPR RNPs to edit each target gene, the cells were expanded in parallel, stained with CFSE, and restimulated in the 4 different suppressive conditions+vehicle. Cells were analyzed by flow cytometry to assess effects of each target gene on proliferative capacity in each suppressive condition. Results are summarized in FIG. 6.

Genes were identified as having a significant role for a specific condition using a cut-off of FDR adjusted p-value<0.05. As expected, ADORA2A, TGFBR1 and TGFBR2, FKBPlA, and PPIA confer resistance in the adenosine, TGFB, Tacrolimus, and Cyclosporine conditions, respectively. PDE4C and NKX2-6 are found to confer relatively selective resistance in the adenosine condition, and NFκB2 is found to increase resistance in the calcineurin inhibitor (tacrolimus and cyclosporine) conditions. TMEM222, while scoring very highly in the screens, does not increase proliferative advantage in this arrayed validation (dots are individuals replicates, black vertical lines are the mean, *p<0.05, **p<0.01, ***p<0.001 and ***p<0.0001 for unpaired Student's t-test).

Using these large arrayed knockout experiments, we also identified that a number of these gene targets confer predicted resistance across diverse suppressive conditions, such as PFN1, FAM49B, and CBLB, and identified new genes that confer more selective resistance. For instance, in addition to showing that ADORA2A and FAM105A knockout confer adenosine resistance as predicted from previous work, we find that genes not previously known to affect adenosine responsiveness, PDE4C and NKX2-6, conferred strong and relatively selective resistance to adenosine (CGS-21680) suppression. Interestingly, while TGFBR1 and TGFBR2 ablation conferred strong resistance to TGFB suppression, we also observed crosstalk with the adenosine suppressive condition—knocking out these TGFB receptors generated resistance to adenosine suppression as well.

We also observed selective resistance to tacrolimus and cyclosporine when their known targets FKBPlA or PPIA were knocked out, but we also found selective resistance to both drugs when NFκB2 was targeted.

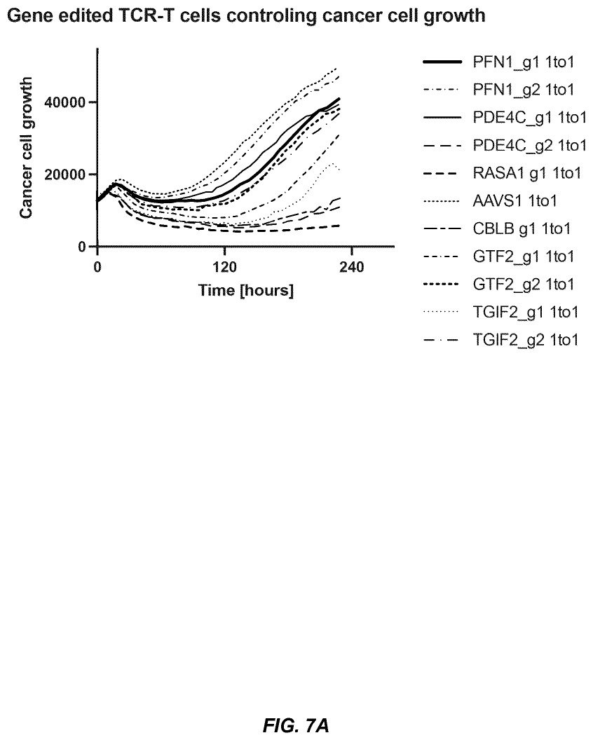

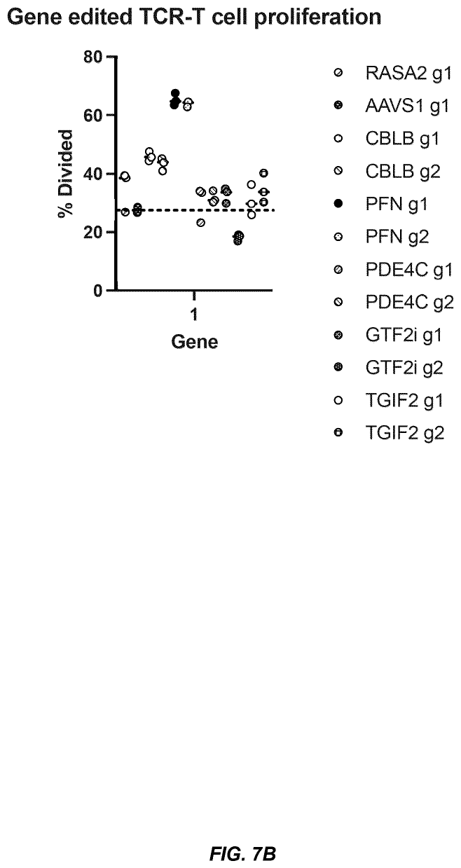

RASA2, CBLB, PFN1, PDE4C, GTF2i, and TGIF2 genes were also targeted to evaluate effects on cancer cell killing (FIGS. 7A and 7B). Primary T cells were transduced with the NY-ESO1 TCR and CRISPR-edited for each of the genes, with two different sgRNAs employed for most of the genes. AAVS1 safe harbor was targeted as a control locus. The edited cells were then co-cultured with A375 cancer cells expressing the cognate peptide on matched MHCI, and the resulting cancer cell killing was measured with the Incucyte live-cell imaging system (FIG. 7A). The TCR-T cells were also tested for their proliferative capacity in response to stimulation (FIG. 7B). The data presented in FIG. 7A indicated that all the target genes tested conferred a killing advantage when knocked out in tumor-antigen-specific T cells. The top-most dotted line in FIG. 7A shows TCR-T cells edited with the control sgRNA. All the other data points for sgRNAs to target the other genes fell below that line, demonstrating better tumor control. These results thus further support that editing any of these target genes boosts tumor cell killing. The data in FIG. 7B indicate that editing nearly all of these target genes conferred a proliferative advantage over the control-edited cells. Taken together, these data further support targeting RASA2, CBLB, TGIF2, GTF2i, PDE4C, or PFN1 in order to boost T cell therapies anti-cancer potential.

In summary, the validation experiments described in this example provide further data supporting targeting selective and pan-suppressive resistance genes.

### Methods

Isolation of Primary T Cells from Healthy Donors

Leukopaks from deidentified healthy donors with Institutional Review Board (IRB)—approved consent forms and protocols were purchased from StemCell Technologies (Catalog #200-0092). For screens, residuals from leukoreduction chambers after Trima Apheresis (Blood Centers of the Pacific, San Francisco, CA) from healthy donors were used. Primary Human T cells were isolated using EasySep Human T cell isolation kit (Cat #17951) according to the manufacturer's protocol using the EasySep magnets. The cells were seeded in appropriate culture vessels and activated with ImmunoCult (Stem Cell Technologies, Cat #10971) at 12.5 μ/ml. Cells were kept in culture at a 1 million/mL density throughout, and cultured with IL2 at 50 IU/mL. Cells were cultured in X-Vivo-15 media which was supplemented with 5% Fetal Calf Serum, 50 μM 2-mercaptoethanol, and 10 mM N-Acetyl L-Cysteine. PBMCs were frozen at 618 5×10⁷ cells per vial using Bambanker (Bulldog Bio) serum-free cell freezing medium.

### Pooled CRISPR-KO Screens Under Suppressive Conditions and Validation of Hits

Pooled CRISPR-KO screens were performed as previously described (Shifrut et al, 2018, supra). Briefly, isolated T cells were stimulated as above and 24 hours later they were transduced with a lentiviral pool to express the genome-wide Brunello sgRNA library (Doench, et al. Nat. Biotechnol. 34, 184-191, 2016). Twenty four hours after transduction, T cells were washed once with PBS, electroporated with Cas9 protein and expanded in culture as above. On Day 14, T cells were stained with CFSE and stimulated with ImmunoCult in the presence of either Tacrolimus (TOCRIS Cat #3631—final 50 nM), Cyclosporine (TOCRIS, Cat #1101—final 50 nM), CGS-21680 (TOCRIS, Cat #1063—final 20 μM) or TGF-01 (Biolegend, Cat #781802—final 10 ng/ml). For the Treg condition, matched donor CD4+CD127lowCD25+ Tregs were isolated on Day 0 using magnetic enrichment (STEMCELL Cat #18063), stimulated with anti-CD3/CD28 and expanded in culture until mixed at 1:1 ratio with the CFSE stained effector T cells. For all screens, 3 days after re-stimulation, stained T cells were sorted to CFSE high and low populations, lysed and genomic DNA was prepped for next-generation sequencing as previously described (Shifrut et al., 2018).

Screen hits were identified using MAGeCK v0.5.9 using paired analysis with default parameters. For tacrolimus and cyclosporine only dividing cells were collected and compared to the undivided cells from the matched donors. Guides with a read count of under 50 in more than 80% of the samples were filtered out. To find shared hits, gene-level log 2 fold-change values were scaled to obtain z-scores. Genes above a z-score of the 95% percentile (zs>1.54) were defined as hits for the shared hits analysis to generate FIG. 1b and FIG. 2a.

### Example 2. RASA2 Ablation Confers T Cell Resistance to Multiple Inhibitor Cues

We also use unbiased genetic screens under the various immunosuppressive conditions as above to examine the effects of RASA2 ablation on T cell resistance to multiple inhibitory cues. These studies showed that ablation of RASA2 enhanced sensitivity to antigen and improved both effector function and long-term persistence of CAR-T and TCR-T cells. We also showed that RASA2-deficient antigen-specific T cells enhance tumor control and extend survival in multiple preclinical models of T cell therapies against both liquid and solid tumors.

Screens were performed as described in Example 1. Analysis of shared hits (z-score>1.5, methods) between the different screens converged on a core set of two candidate resistance target genes: TMEM222, and RASA2 (FIG. 1b). We had previously identified RASA2 as a gene target that boosts T cell proliferation and in vitro cancer cell killing capacity when it is knocked out (Shifrut et al, 2018, WO2020/014235). In view of the discovery that RASA2 ablation also promoted T cell proliferative capacity under multiple immunosuppressive environments, we then focused on characterizing the effects of RASA2 ablation and testing the performance of RASA2 knockout (KO) T cells in a range of preclinical models of adoptive cell therapy

RASA2 is a Ras-GTPase activating protein (RasGAP), predicted to suppress Ras signaling, with no known function in T cell biology (King et al, Sci. Signal. 6, rel, 2013; Chen, et al., Mol. Cell 45, 196-209, 2012; Arafeh, et al., Nat. Genet. 47, 1045 1408-1410, 2015). In these screens, RASA2 was unique among the RasGAP family in inhibiting T cell proliferation as evidenced by multiple RASA2 targeting guides in multiple donors being enriched in the dividing T cells (FIG. 1c-d). In contrast, guides targeting the Ras guanine nucleotide exchange factor RASGRP1 were depleted from dividing T cells, confirming its known role as a positive regulator of TCR signaling (Priatel, et al., Immunity 17, 617-627, 2002). (FIG. 1d). Targeted RASA2 ablation with individual CRISPR guides reproduced the proliferative advantage observed in the screens in all 4 suppressive molecule conditions (FIG. 1e). We also tested whether RASA2-deficient T cells could demonstrate increased in vitro killing of cancer cells under these immunosuppressive conditions.

RASA2 ablation boosted cancer cell killing by TCR-T cells compared to control-edited T cells across the range of suppressive conditions (FIG. 1f). A co-culture suppression assay with Tregs further confirmed RASA2 ablation renders effector T cells resistant to Treg-mediated inhibition of proliferation (FIG. 1g). This resistance to suppression was also evident in cancer killing assays performed in the presence of Tregs (FIG. 1h). RASA2-deficient effector T cells maintained their robust cytotoxic function while control-edited T cells were unable to control tumor cell growth in the presence of suppressive Tregs. These findings indicate that RASA2 is a negative regulator of T cell proliferation and cytotoxic function and that RASA2 ablation can confer resistance to multiple forms of T cell suppression.

RASA2 is a TCR Stimulation-Dependent Negative Regulator ofRas Signaling

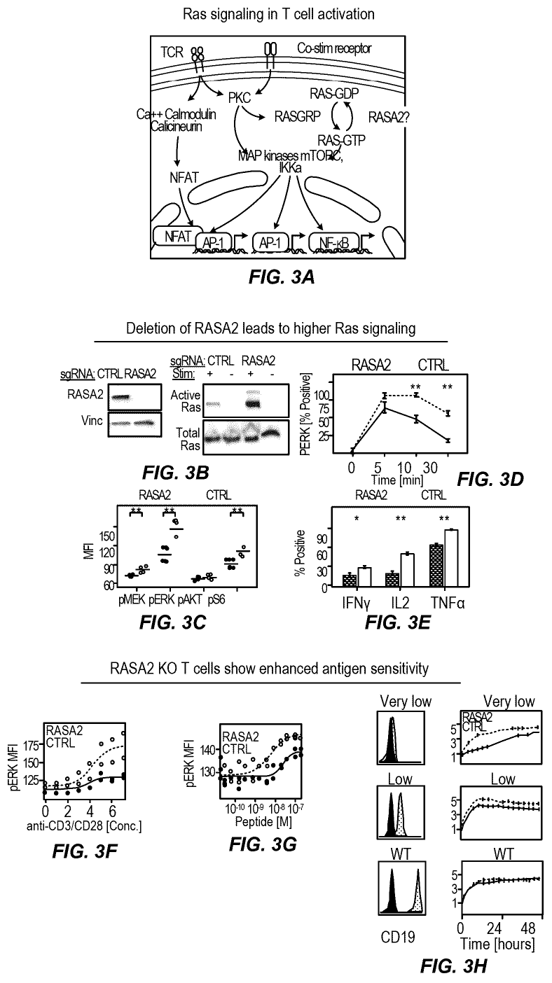

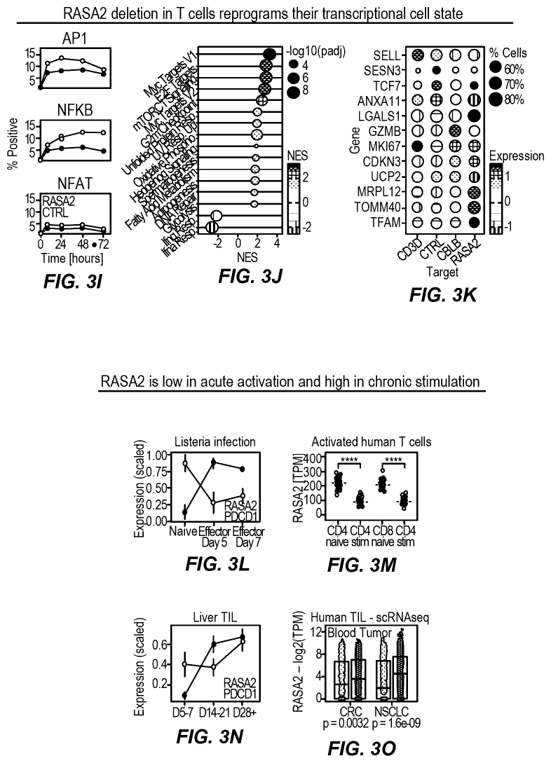

We nexts sought to define how RASA2 ablation modulates downstream signaling events in primary human T cells. RASA2 is a member of the GAP1m family of RasGAPs that inactivate Ras by stimulating its GTPase activity (King et al. Sci. Signal. 6, rel, 2013). Thus, RASA2 is predicted to attenuate Ras signaling, a major intersection for multiple pathways in T cells that control cell activation, proliferation, and differentiation (Kortum et al, Trends Immunol. 34, 259-268, 2013; Lapinski et al., Am. J Clin. Exp. Immunol. 1, 147-153, 2012) (FIG. 3a). In support of its role as a RasGAP, RASA2 ablation increased total active Ras levels compared to control in a TCR stimulation-dependent manner in both Jurkat T cells and primary human T cells (FIG. 3b). This dependence on TCR stimulation was confirmed by elevated phospho-ERK (pERK) signaling, activation (CD69), and proliferation (CFSE) in stimulated RASA2 KO T cells specifically, with no consistent change in baseline levels evident. We wanted to confirm that RASA2 ablation does not cause unregulated T cell proliferation, which would make it less safe as a gene editing target in T cell therapies. In the absence of TCR stimulation, the viability of both control and RASA2 KO T cells steadily declined, and withdrawal of IL2 enhanced this decline. RASA2 ablation resulted in higher levels of stimulation-induced phosphorylation of key RAS signaling mediators, such as MEK and ERK in the MAP kinase pathway, as well as the 40S ribosome protein S6 downstream of mTOR (FIG. 3c). While RASA2 KO T cells followed similar overall kinetics of MAP kinase signaling as control cells, they reached a higher peak amplitude of pERK and pMEK levels (FIG. 3d). Additionally, we detected higher levels of multiple effector cytokines in RASA2-deficient T cells compared to control T cells in response to TCR stimulation (FIG. 3e). Together, these results demonstrate that in TCR stimulated T cells, RASA2 ablation boosts a cascade of key signaling pathways to promote more potent effector functions. However, RASA2 ablation does not cause unregulated proliferation as the effects were found selectively in TCR-stimulated T cells and the knockout cells remained cytokine-dependent.

TCR-T and CAR-T Cells with Ablation of RASA2 are More Sensitive to Low Antigen Levels

We next tested whether RASA2 ablation in T cells amplifies sensitivity to lower levels of target cognate antigen. T cells with RASA2 ablation had higher levels of pERK and activation levels compared to control T cells across a wide range of anti-CD3/CD28 concentrations (FIG. 3f). To measure this antigen sensitivity with a more physiological stimulus, antigen-specific T cells were co-cultured with T2 cells preloaded with increasing concentrations of the cognate NY-ESO-1 peptide. This assay confirmed RASA2 ablation leads to higher levels of pERK across a range of peptide concentrations, effectively sensitizing T cells to lower levels of antigen (FIG. 3g). Increased antigen sensitivity could be particularly important in engineering T cells that are able to detect and kill cancer cells with low target antigen expression (Feucht et al., Nat. Med. 25, 82-88, 2019; Majzner, et al., Cancer Discov. 10, 702-723, 2020). To test this, T cells were engineered to express a CAR targeting the CD19 surface protein and edited to disrupt either RASA2 or a control locus. We used a CD28-based CD19 CAR, which has been reported to be the most sensitive CAR, to see if we could even further boost sensitivity to low antigen targets with RASA2 ablation. These CAR-T cells were co-cultured with cancer cells engineered to express a range of CD19 levels, and cancer cell killing was assayed by annexin staining. While both RASA2 KO and control CAR-T cells kill leukemia cells with high antigen levels effectively, RASA2 KO CAR-T cells showed the most significant killing advantage over the control T cells when cultured with leukemia cells expressing the lowest CD19 levels (FIG. 3h). Collectively, these data suggest that T cells with RASA2 ablation are sensitized to low antigen levels, enhancing their ability to detect and kill antigen-dim cancer cells.

### Ablation of RASA2 Promotes Transcriptional Reprogramming of Engineered T Cells

We next profiled the transcriptional events downstream of RASA2 ablation. First, to assess transcriptional programs key to T cell activation, we used a set of Jurkat T cell transcriptional reporter systems. These reporter lines are engineered with response elements for activator protein 1 (AP-1), nuclear factor of activated T cells (NFAT), and nuclear factor kappa B (NFκB) driving the expression of an mCherry fluorescent reporter. These reporter lines revealed that RASA2 ablation significantly increased TCR stimulation-induced transcriptional activity of AP-1 and NFκB, and to a lesser extent NFAT, consistent with the established downstream transcriptional effects of Ras and MAPK signaling pathways (FIG. 3i). To profile transcriptional changes systematically in primary T cells downstream of RASA2 ablation, we performed RNA-Seq analysis on either RASA2 or control edited antigen-specific T cells after 48 hours of co-culture with target cancer cells. Two of the most upregulated genes in RASA2 KO T cells were genes known to attenuate Ras signaling, DUSP6 and SPRED2, which are likely upregulated as a feedback mechanism in the setting of elevated Ras signaling (Wakioka et al, Nature 412, 647-651, 2001; Li, et al., Nat. Med. 18, 1518-1524, 2012). Gene set enrichment analysis highlighted multiple key pathways upregulated in RASA2 KO T cells, such as those associated with cell cycle, transcriptional activity, and cell metabolism (FIG. 3j). Interestingly, given the importance of metabolic state to T cell function, RASA2-deficient T cells showed increased expression of genes involved in oxidative phosphorylation and in glycolysis. To test whether these metabolic changes are generally common to hyper-activated T cells, we analyzed a single-cell RNA-seq (scRNA-Seq) dataset we previously generated in CRISPR-perturbed primary human T cells (Shifrut et al, 2018). We compared genes differentially expressed in RASA2 KO T cells with T cells lacking CBLB, a well characterized negative regulator of TCR signaling. While RASA2 and CBLB ablation both increased levels of GZMB, MKI67, and CDKN3, and decreased CD62L and TCF7 (FIG. 3k), our analysis revealed that ablation of RASA2 also induced a unique gene signature. This signature included differential expression of core genes involved in mitochondrial activity, such as MRPL12, TOMM40, TFAM, and UCP232,33. Metabolic regulation by RASA2 was underscored by a strong negative correlation between genes driving oxidative phosphorylation and RASA2 expression across thousands of transcriptional datasets from immune cells (data not shown). Overall, our analysis of the transcriptional state of RASA2 KO T cells revealed a heightened effector memory state coupled with a higher oxidative phosphorylation state, typically associated with central memory T cells (Chapman, et al, Nat. Rev. Immunol. 20, 55-70, 2020).

As RASA2 has no previously described roles in T cell biology, we next evaluated its endogenous transcriptional regulation in T cells. Analysis of our previously published scRNA-Seq dataset (Shifrut et al, 2018) revealed that RASA2 is downregulated following stimulation in human T cells. Further analysis of two published RNA-Seq datasets of acute bacterial infection in mice (Philip et al, Nature 545:452-456, 2017) and a large cohort of in vitro activated human T cells (Schmiedel, et al., Cell 175, 1701-1715.e16, 2018) confirmed that T cell stimulation acutely downregulates RASA2 expression levels (FIG. 3, l-m). This acute endogenous reduction of RASA2 after stimulation may give T cells a window of heightened effector function, while genetic ablation of RASA2 may amplify this phenomenon through complete and enduring loss of RASA2. Additionally, we asked whether RASA2 plays a role in T cell exhaustion and dysfunction through analysis of external datasets. Consistent with a checkpoint role in regulating T cell function, RASA2 was upregulated in mouse T cells exposed to chronic infection (Pauken et al, Science 354:1160-1165, 2016) or to repeated antigen stimulation5, as well as in tumor-infiltrating T cells (FIG. 3n). Published scRNA-seq datasets from human patients (Zhang, et al, Nature 564, 268-272, 2018; Guo et al., Nat. Med. 24, 978-985, 2018) also revealed higher RASA2 levels in tumor infiltrating T cells compared to peripheral T cells, suggesting a potential role for RASA2 in dampening T cell responsiveness in the tumor microenvironment (FIG. 3o). These observations suggest that RASA2, which is downregulated during acute stimulation, can be induced in chronically stimulated T cells to serve as an intrinsic signaling checkpoint.

RASA2 Ablation Boosts T Cell Persistence and Cancer Cell Killing Capacity after Repeated Tumor Exposures

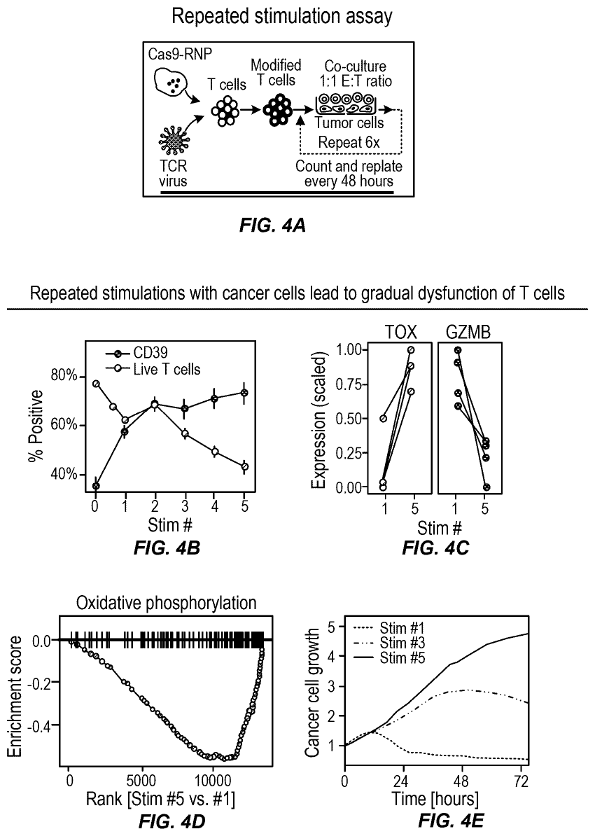

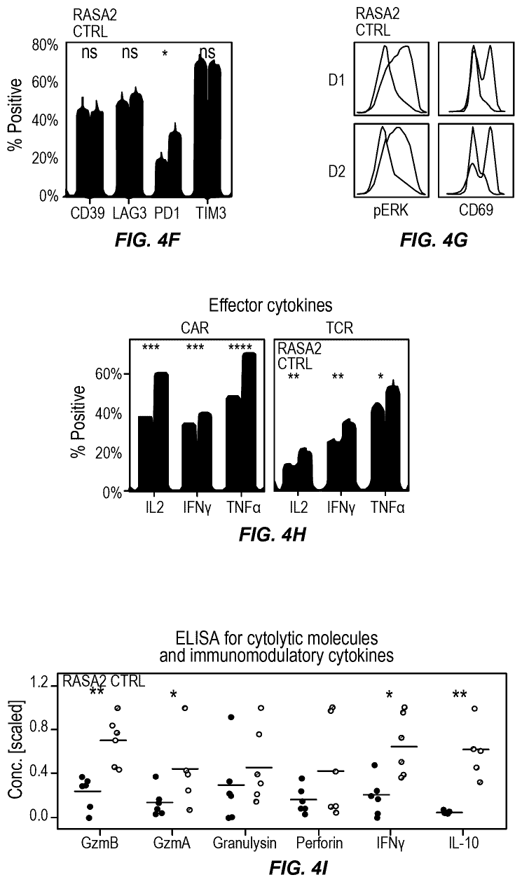

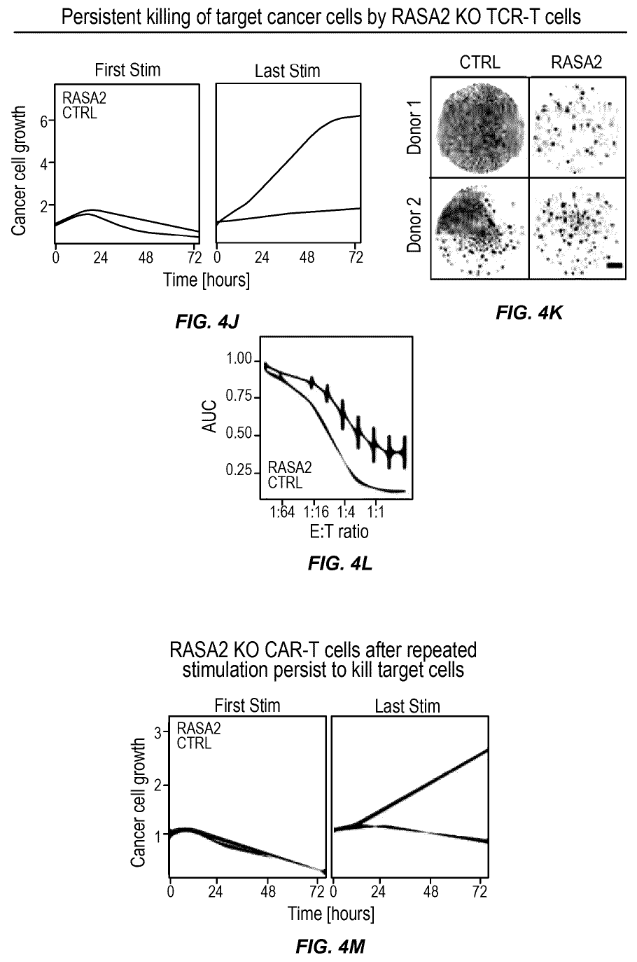

We next tested if ablation of RASA2, which we found to be upregulated in T tumor infiltrating T cells, would ameliorate chronic antigen exposure-induced T cell dysfunction. To that end, we established a repetitive stimulation assay where antigen-specific T cells are co-cultured with fresh target tumor cells at 1:1 effector to target (E:T) ratios repeatedly every 48 hours (FIG. 4a). This repetitive stimulation assay showed a relative enrichment in NY-ESO-1 specific T cells, a decline in T cell viability and activation levels, and an induction of CD39, a marker for exhausted T cells40 (FIG. 4b). Furthermore, TOX expression was increased while expression of GZMB and genes associated with oxidative phosphorylation decreased after repetitive stimulation, suggesting a dysfunctional T cell state5 (FIG. 4c-d). Indeed, T cells gradually failed to control the expansion of cancer cells after repeated exposures (FIG. 4e). Using this repetitive stimulation assay, we noted that ablation of RASA2 partially counteracted the observed decline in T cell viability (data not shown). Levels of canonical T cell exhaustion markers (LAG3, PD1, TIM3, CD39) were similar between RASA2 and control-edited T cells after multiple stimulations, suggesting that RASA2 KO T cells were not more exhausted (FIG. 4f). Additionally, RASA2 ablation increased pERK, activation levels, effector memory state, and multiple effector cytokines to higher levels compared to control-edited T cells after repeated stimulations (FIG. 4g-h). This enhanced effector state of RASA2-deficient T cells was confirmed independently using an ELISA assay to measure immunomodulatory cytokines and cytolytic molecules in the supernatant of stimulated T cells (FIG. 4i). Interestingly, among the elevated cytokines we found that RASA2 KO T cells secreted starkly higher levels of IL-10 compared to control cells, an important immunomodulatory cytokine which could play a potential role in their metabolic reprogramming. RNA-Seq analysis showed that RASA2 KO T cells expressed higher levels of cell cycle (VRK1, AURKA, KNL1), fatty acid metabolism (SLC27A2), and mitochondrial genes compared to control-edited T cells after repeated stimulations (data not shown). This increase in mitochondrial gene transcription was further corroborated in an orthogonal measurement of mitochondrial mass by flow cytometry in both CAR- and TCR-T cells lacking RASA2 (data not shown). Overall, these findings suggest that genetic ablation of RASA2 protects T cell viability, activation, and metabolic fitness in the setting of repeated antigen exposures.

Next, we tested whether the cancer-cell killing capacity of RASA2-ablated T cells is affected by repeated exposure to tumor antigen. Although T cells with RASA2 ablation had a moderate advantage in our cancer cell killing assay upon first stimulation, this advantage became more striking after multiple stimulations (FIG. 4j,k). In contrast to control-edited T cells that showed a gradual failure to control the growth of cancer cells with each stimulation, RASA2-ablated T cells maintained their robust killing capacity after multiple stimulations (data not shown). This cancer cell killing advantage was consistent across multiple human blood donors and ratios of effector T cell to cancer cells (FIG. 4l). We next asked whether this resistance to T cell dysfunction with RASA2 loss was replicated in CAR-T cells. RASA2-deleted CD19-specific CAR T cells were co-cultured repeatedly with CD19-expressing cancer cells (data not shown). As seen with the TCR-T cell model, RASA2-edited CAR-T cells continued to kill target cells efficiently following repeated cancer cell exposures, while the control-edited CAR-T cells were unable to control tumor cell growth (FIG. 4m). This persistent killing was consistent using two different CD19+ cancer cell lines and multiple human blood donors (data not shown). This killing advantage after repetitive stimulation was specific as demonstrated by the lack of cancer cell killing when either RASA2 KO or control CAR-T cells were co-cultured with antigen negative cancer cells (data not shown). Collectively, these results show that T cells repeatedly exposed to their target antigen gradually fail to control tumor cell growth, while ablation of RASA2 rescues both TCR-T and CAR-T cells from this dysfunctional state. RASA2-deficient engineered T cells improve in vivo anti-tumor responses in both liquid and solid tumor preclinical models.

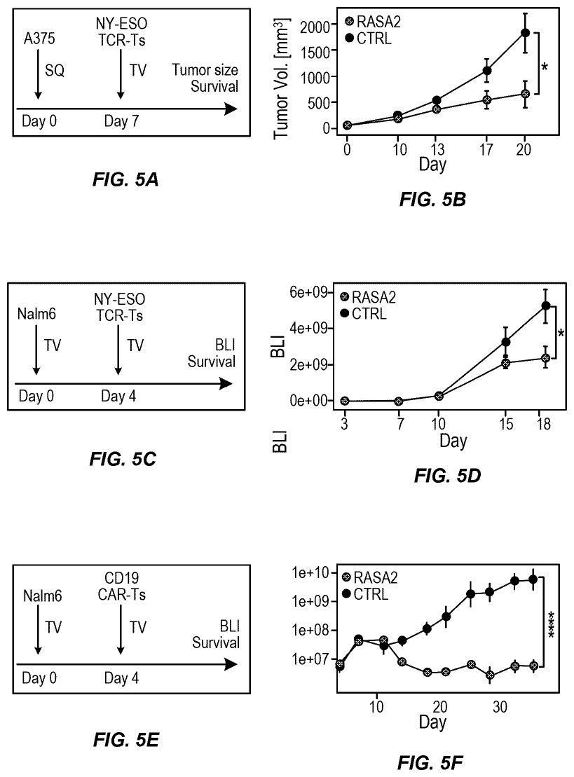

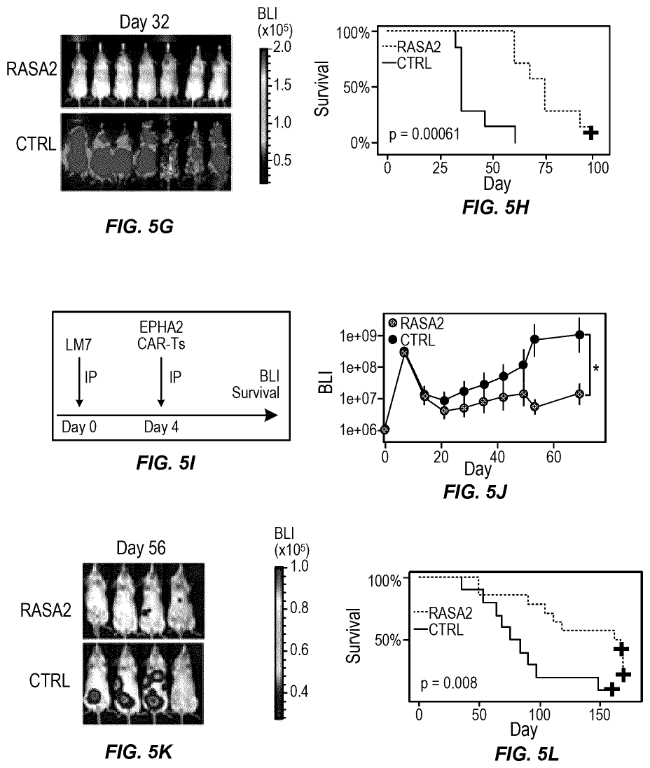

To determine the translational relevance of these findings, we then tested whether ablation of RASA2 would improve the performance of engineered T cells in multiple preclinical models of adoptive T cell therapies. First, A375 melanoma cells, which express NY-ESO-1, were engrafted in the flanks of immunodeficient NSG mice (FIG. 5a). T cells engineered to express the 1G4 NY-ESO-1-specific TCR42 and edited to ablate RASA2 or a control locus were transferred via tail vein injection. Transfer of RASA2-deficient T cells significantly slowed the tumor growth and improved survival compared to mice that received control-edited T cells (FIG. 5b). To test whether RASA2 ablation in TCR-T cells also improved control of liquid tumors, mice were injected via the tail vein with Nalm6 leukemia cells engineered to express NY-ESO-1 (FIG. 5c). In this leukemia model, RASA2-deficient TCR-T cells improvedl0 tumor control, consistent with the results with the A375 melanoma model 252 (FIG. 5d). Thus, RASA2 ablation enhanced efficacy of TCR-engineered adoptive T cell therapies in both liquid and solid tumor models.

To test if this RASA2 KO advantage in vivo is applicable to the CAR-T cell context, we generated CD19-specific CAR-T cells via knock-in of the CD19-28z CAR into the TRAC locus as previously described (Eyquem, et al., Nature 543, 113-117, 2017), with the addition of concurrent disruption of either RASA2 or of a safe harbor control locus (AAVS1). These CAR-T cells were transferred intravenously into NSG mice engrafted with Nalm6 leukemia cells (FIG. 5e). CAR knock-in at the TRAC locus has been shown to reduce T cell dysfunction and increase persistence compared to CAR expressed by retroviral vectors (Eyquem et al, supra). Nonetheless, we found that the RASA2-deficient TRAC CAR-T cells had a striking advantage over the control TRAC CAR-T cells in tumor control, as measured by bioluminescence imaging in cohorts of animals treated with cells from multiple different human blood donors (FIG. 5f,g). This reduced tumor burden resulted in significantly prolonged survival of the mice that received RASA2-deficient TRAC CAR-T cells (FIG. 5h). While all mice injected with the control edited CAR-T cells had to be euthanized by day 60, the majority of mice receiving RASA2 KO 268 human T cells survived past day 60, with a subset demonstrating durable responses beyond 100 days. To assess the effects of adoptive T cell transfer alone on the health of the mice, mice with no tumors were injected with T cells and followed over time. In addition, to assess tumor-antigen stimulated T cells, we treated an additional cohort of mice bearing Nalm6 leukemia with control and RASA2 KO CD19 CAR-T cells to achieve tumor clearance, and observed these mice beyond 120 days. In both of these cohorts, there were no observed differences in mice receiving the RASA2 KO and control T cells by visual inspection and body weight, and RASA2 KO did not alter the blood counts or histopathologic findings of recipient animals in comparison to control T cells (data not shown). Overall, these data demonstrate that RASA2 can be ablated in CAR T cells to improve anti-tumor efficacy and survival with no apparent increased safety risk in this preclinical model.

Finally, given the major clinical challenges in developing CAR-T cell therapies for solid tumors, we tested whether RASA2 KO could also enhance CAR-T cell function in a solid tumor preclinical model. We made use of our previously described intraperitoneal locoregional osteosarcoma (LM7) model44 and T-cells expressing EphA2.CD28z CAR45. The LM7 osteosarcoma cell line was injected into the peritoneum ofNSG mice, followed by injection of T cells engineered to express an EphA2-specific CAR (FIG. 5i). Bioluminescence measurements of tumor burden revealed that ablation of RASA2 in CAR-T cells significantly slowed tumor growth and prolonged survival compared to control CAR-T cells 286 in this model (FIG. 5j-l). In the subset of mice that cleared their tumors, RASA2 KO CAR-T cells were able to clear a tumor rechallenge at Day 174, suggesting they have remarkably durable in vivo function. In summary, we found that RASA2 ablation improved the performance of TCR-T and CAR-T cells against a range of preclinical models of both liquid and solid tumors highlighting its promising translational potential across multiple indications.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for the contents for which they are cited.

TABLE 1
Positive Hits from Screens that Model Immuno-
suppressive TME Factors

| Gene | sgRNA |
|---|---|
| ALAS1 | TGGTGCAGTAATGACTACCT |
| ALAS1 | TCGAATCCCTTGGATCATGG |
| ALAS1 | ACAGATCAAAGAAACCCCTC |
| ALAS1 | CCACAATCTTGGGGACTGAG |
| AMBRA1 | GGAGCTGGGGACCTGCCAAG |
| AMBRA1 | TTTCTCGGTGATACCTAGAA |
| AMBRA1 | CCATAATATCTATATTACGG |
| AMBRA1 | GGATGATCCAGTATCTCTCA |
| ANKRD32 | GCTTATCAGTTCTAACAAGG |
| ANKRD32 | GCATGAAGAACGCATACAGG |
| ANKRD32 | CATGGCTATTAAGACAGATG |
| ANKRD32 | AGAGACCATGTATAGAACCC |
| ARHGAP15 | ATGCATTGCTTTACAAACCA |
| ARHGAP15 | CCAGAAAGTGTGGATTTGTG |
| ARHGAP15 | TTCAGTGACCTTCCCGACAT |
| ARHGAP15 | ACTTAGCAATTCAGTGCTAG |
| C15orf40 | CGGGCAACACCCAATACTCG |
| C15orf40 | CCTGTGGCAGTTGATCCTAA |
| C15orf40 | ATAGCCATCCATGCAAAACC |
| C15orf40 | GAAGGCTGGTGCGACGACCA |
| C3orf33 | TTAACCAGCAAAGCACCACG |
| C3orf33 | TGGAACCATAGTAATTGGGA |
| C3orf33 | AATATGATTCTAAAATCTAC |
| C3orf33 | AGAAGAAATGTTAAACTACG |
| C8orf44 | GGGAGCACTTCGAAAACCAC |
| C8orf44 | CCTCCCATGAGGGAAAGTGT |
| C8orf44 | AGAAGCAGAAAAGGCAAAAG |
| CARKD | CATCCCTGTTGTCATCGACG |
| CARKD | CCTGTGTTGTCATAGCTCAG |
| CARKD | GGAAGAATAGGCGTAGTTGG |
| CARKD | CGCACTGGCACAGAACACGT |
| CD300LB | ACGCAGATGTTTACTGGTGT |
| CD300LB | GGGAGACCTACATTAAGTGG |
| CD300LB | CCTCATTGAAACCAGAGGGT |
| CD300LB | ATAGTGGCATTGAACCGTCA |
| CENPB | GTACCATAGACTGGTCTCGG |
| CENPB | CATAGCCGCCTGCTTTCGTG |
| CENPB | GGGATCCTGGCCCTCTAGCG |
| CHL1 | TTTAAACTTACTGTCAACTG |
| CHL1 | CATTCAGGATCCCAAACGAG |
| CHL1 | TGGAGAAAATTACGCTACAG |
| CHL1 | AGATCTATACTTCGCAAACG |
| CHST3 | CACTGCAAGAACCGCCGCTG |
| CHST3 | ACTCAGTTCATGTTCCGCCG |
| CHST3 | GAAGAACTCGCCCACGAACG |
| CHST3 | GAGCACGTCGCGGTACACCA |
| CLEC4M | GAAGAAGATCCAACAACCAG |
| CLEC4M | GACCCCAGCCAAGAGCATGA |
| CLEC4M | GGAGATCTACCAGGAGCTGA |
| CLEC4M | TCCAGTCCTTGGGACAGTGG |
| COL15A1 | CTGTACGATCCTTACCACAG |
| COL15A1 | CTCCTCACAGTTCACGAGGA |
| COL15A1 | TGGCGAAGAGCACGCCACCA |
| COL15A1 | AAAGTTCCATGTCTTCAAAG |
| COL25A1 | TAAGCACTACCTTAATCAGG |
| COL25A1 | AAACCAACGACCTCCAGGCG |
| COL25A1 | GGGTCCACCTGGTCAAAAAG |
| COL25A1 | ATTTGTGACAGGGTGAACGG |
| CORO1A | GTGCAGTGTTCGTGTCGGAG |
| CORO1A | CCCAGACACGATCTACAGTG |
| CORO1A | GCCACAGAGGTAGACGATGT |
| CORO1A | GTGCCTTTAGACTGGACGTG |
| CUL3 | ATCCAGCGTAAGAATAACAG |
| CUL3 | GGTGTATTAGGGATCATCTA |
| CUL3 | ATAACTTGTACATGCAACCA |
| CUL3 | GAGCATCTCAAACACAACGA |
| CWC27 | CCTGGTTTCATAGTCCAAGG |
| CWC27 | ACTGGAACAGAACTGAGATG |
| CWC27 | TTTAATTTGATAGGTTACAG |
| CWC27 | GAGATATTGACATAGAGTTG |
| CYC1 | GGTGGGCGTGTGCTACACGG |
| CYC1 | AGCTATCCGTGGTCTCACCG |
| CYC1 | AAGGCCAGACTTCGACGACA |
| CYC1 | AAAACCATACCCCAACAGTG |
| DOK2 | GTAGGGCCAGTCGTACAGCT |
| DOK2 | CCCCGCGCGACTCACGGGGA |
| DOK2 | GTCCGACCCTCCATACAGTG |
| DOK2 | GGAGGGTATAGGACCCCCGC |
| DUSP4 | AGCGCACGTTGACCGAACCT |
| DUSP4 | GCGCTCCGGCCTCTACTCGG |
| DUSP4 | TCAGTACAAGTGCATCCCAG |
| DUSP4 | TGGGACCCCACTACACGACC |
| ENG | ATGAGCCAGGACACGTAGGG |
| ENG | CGTACTCCAGCCTTGGTCCG |
| ENG | AGGAGTGGTCTGGATCGGTG |
| ENG | ACCACTAGCCAGGTCTCGAA |
| FAM49B | AGCTGGCCACGAAATACGAG |
| FAM49B | TATGAGGATTAACAATGTAC |
| FAM49B | CCAGCCTACAGAGTCTGAGA |
| FUBP1 | CAAAAATTGGAGGTGATGCA |
| FUBP1 | TGATTGTAACAGGAACGGGC |
| FUBP1 | ATGATGGGACAACACCCGAA |
| FUBP1 | GAGAAGTTCGGAATGAGTAT |
| GAB3 | TCATGTTGTCTAAACCAGAG |
| GAB3 | TTCCCGTACATTCTACCTGG |
| GAB3 | ACCTCAGCGAGTGTGCAGTG |
| GAB3 | GACCAGCTATCACATCTGGT |
| GLRX | AATCTCGTTAGTGTGGTTGG |
| GLRX | CACCTGCCCGTACTGCAGGA |
| GLRX | AGATTATTTGCAACAGCTCA |
| GLRX | GTCAATTGCCCATCAAACAA |
| GREB1L | GTCGCGGAGTGACGCCATGG |
| GREB1L | AGCTTGAGTCAATTAACCGG |
| GREBIL | ACTCCCATGTGGAACTAACG |
| GREBIL | TTAGCTGCGGTTGATAAATG |
| GTF2H2 | ATTAGATCATAAATATTAGA |
| GTF2H2 | AACGTCTTTGAAGAAAGCTG |
| GTF2H2 | CATCTACTACCACATAAAGG |
| GTF2H2 | GGCTTTCATCTAAAATAACA |
| HAUS1 | TAAGAATTACCTAGCTAGCG |
| HAUS1 | TGATAATCGTCGTCAGAACA |
| HAUS1 | CAGTGCTAGAGAGATTGGCG |
| HAUS1 | GCAAGTGAATACGAGTCAGA |
| HIST1H2AD | GGAGTCCGGCCCGCGAAGAG |
| HIST1H2AD | ACGCGGCAAGCAAGGCGGAA |
| HISTIH2AD | GCTCGGAGTAGTTGCCCTTG |
| HISTIH2AD | CAACTACTCCGAGCGAGTCG |
| HIST1H2BC | CGACATATTTGAGCGCATCG |
| HIST1H2BC | GAAAGAATTCATGATGCCCA |
| HIST1H2BC | CTTGGAAGAGATGCCAGTGT |
| HIST1H2BC | ACACAGAGTAACTCTCCTTG |
| HOXA10 | CTACTGCCTCTACGACTCGG |
| HOXA10 | GACCAAAAAAGAGTTCGCGG |
| HOXA10 | GGCGGTTACTACGCCCACGG |
| HOXA10 | AGATCGAAACCGCGCCCCGG |
| IGFBP4 | CTGAATACAGACAAGGACGA |
| IGFBP4 | CACACACTGATGCACGGGCA |
| IGFBP4 | ACAGGCCGGGCATCCTCCCG |
| IRF2BP2 | GCCCCCTAAGATCAACGGAG |
| IRF2BP2 | TTCACCGAACCCGTCTGCCG |
| IRF2BP2 | CAACGGCTTCTCCAAGCTAG |
| IRF2BP2 | GGCCGACAGCCTGTCCACCG |
| IYD | TCACAGACCTCAAGAAACTG |
| IYD | TCATAACCACTATCCTGAGA |
| IYD | TGGGTCCTTCACAACCACGA |
| IYD | CAAGATTCGAAAGATCATTG |
| KCNK4 | GACAGCACTCTTACTAGCTC |
| KCNK4 | ATAGTGACGCTTACCACCGT |
| KCNK4 | GCCAGTAGGATCCCAAACAG |
| KCNK4 | ATATAGCAGAACACGAACGT |
| KDM6B | GCAGTCGGAAACCGTTCTTG |
| KDM6B | GCTGGACGAATCCATTCGCA |
| KDM6B | GGTGCTAGAAGAGATCAGCC |
| KDM6B | GACAAAAGTACTGTTATCGG |
| LICAM | GAGTAGCCGATAGTGACCTG |
| LICAM | GCATGCGTACTATGTCACCG |
| LICAM | CACAATGGTGACCCAATGTG |
| LICAM | CTTGGGGACAGTGACAAGTG |
| LAMA3 | GAGATCCAACTGTCACTCGG |
| LAMA3 | CTGACAAGAGTCACCAGCGT |
| LAMA3 | TGTGGTCGAGTATTCCACGG |
| LAMA3 | GAGCGGCAGGTATCACATCG |
| MBTD1 | TTATGTGACAGGCACCTATG |
| MBTD1 | ATTGGTTGGTCTCGAAGCAT |
| MBTD1 | GTGTCGAACACGAGTAGCAG |
| MBTD1 | AGCTTGATACTGAGCATATG |
| MRPL17 | TCGGTACAAAGATCAAACTG |
| MRPL17 | GGCCCATACGGCGAAATACG |
| MRPL17 | ACGAACGCATCGAGGCACCA |
| MRPL17 | TAACGAACGAGCCATGCGCA |
| MRPL33 | TGAGAATGGTGAGCGAAGCT |
| MRPL33 | GCCCCACTTACAGAAGACCG |
| MRPL33 | ACACCAAGAGAAACCGACTG |
| MRPL33 | TAGAAACATTCTGGTGAGAA |
| MTIF2 | ATGGATTGGAATGACTATTG |
| MTIF2 | GATCAAAGAAGTGATAACGA |
| MTIF2 | CCCCGCATTTACCGTAAGTG |
| MTIF2 | GCCCATTATAGTAACAACTG |
| MYO1H | TCCAGCAACTGTTAATTGAG |
| MYO1H | AGAGGTATTTATACAGCTGG |
| MYO1H | TGGCCGAGTTAGAAAACCGG |
| MYO1H | GTACATCGGCTACAAACCCG |
| NEFL | CAAGAACATGCAGAACGCTG |
| NEFL | TCTTGGCCTTGAGCAGACGA |
| NEFL | CCTGCGTGCGGATGGACTTG |
| NEFL | AGCGCGCAAAGGCGCCGACG |
| NFKB2 | GGGACCAGCCAAGATCGAGG |
| NFKB2 | CTGCAACTGAAACGCAAGCG |
| NFKB2 | CCCACTCCATAGAATCTCCG |
| NFKB2 | ACTCGACTACGGCGTCACCG |
| NMT1 | AGGACAACAGCTACAACCGG |
| NMT1 | GCATGTACATACCCAGCTTG |
| NMT1 | GGGTTCGAGTGGTCTCAAGT |
| NMT1 | GGGCTTTGGTAGTACCACCC |
| ORC6 | GACCTACTTACCCTGTCCAA |
| ORC6 | TCTTCCCCAGACACAGCAAG |
| ORC6 | GAGGCCGACACACTTCACCC |
| ORC6 | TACAGCTAAACTGTACAGCT |
| PCBP2 | GTGTGTCAAACAGATCTGCG |
| PCBP2 | CAAGATCAAGGAAATACGAG |
| PCBP2 | AGTTGGCAGTATCATCGGAA |
| PCBP2 | CACGTATCAACATCTCAGAA |
| PCGF1 | CCAGTCCCGAGGTTTGGACC |
| PCGF1 | CCACGAAGTAGCCGGCGCAT |
| PCGF1 | GCTCATCATAGCGATAGTAG |
| PCGF1 | CCTTGCACCTCGTTCCGTAG |
| PDCD6IP | CGTCCGCTGGACAAGCACGA |
| PDCD6IP | CACACTTGTGAAATCTACCC |
| PDCD6IP | AATTTAAGGAACGTTGGCAA |
| PDCD6IP | CTTAAGTCGAGAGCCGACCG |
| PDCL | GAAGGCATCTCAGTTAACAC |
| PDCL | GCAGTACCGGAAGCAGCGAA |
| PDCL | GACCACGAGGACAAGGACCG |
| PDCL | GGAGTTTGCCATAATGAATG |
| PIWIL4 | TGTCCATGTACCAAATTGGA |
| PIWIL4 | AATCTGGAATATATGTCACA |
| PIWIL4 | GTCGCTACATAAAATCAACC |
| PIWIL4 | CTGCTTGTAGTAATCCACAT |
| PLGLB2 | ACTGAACAGTGAAGGCCCCT |
| PLGLB2 | GGTATTCACATAGTCATCCA |
| PLGLB2 | ATGTGCAGCAAAATGTGAAG |
| PLGLB2 | TGTCACTAAGAAGCAGCTGG |
| POTEJ | AAGCACGGAAGTACTCACGT |
| POTEJ | GTGGTATCTCGGCTCCACGA |
| POTEJ | GTAGATAGCGTAGTGTAGAG |
| POTEJ | GTTGTCAATGACGAGCACAG |
| PPP2R5D | AAGAGGTTCACTGAAAACTG |
| PPP2R5D | AGGGACTTGACCTTGTGAAG |
| PPP2R5D | GGCTCCGGGCTTATATCCGT |
| PPP2R5D | CAGCAAAATCAAGTACTCAG |
| PTPRG | GGTCCCTTGGAATATTCGAG |
| PTPRG | TCCACTATTTCGCTACACGG |
| PTPRG | CTTTCATTAGGGACCACTCG |
| PTPRG | TAAAAAGCCTATGTCCCGCG |
| RFPL1 | GCCAGGGGAAAAGTGCACAA |
| RFPL1 | TCTGTGTGATGCACCCACTT |
| RFPL1 | GACAGCGCATCCACACTCCA |
| RFPL1 | AATCTGTTCACCGCAAAGGG |
| RNF13 | TGGAGGCACTATGGGTTCAC |
| RNF13 | ATACCTGTACTTACTGTCGT |
| RNF13 | GGAATTAGGTAGTATTCCAA |
| RNF13 | TATAACCAAATCTTGCAGGG |
| RNF185 | CATCTTACCTGATGTAAACA |
| RNF185 | GTGGCCACACAGGCTGATGA |
| RNF185 | GGAGACCAGACCTAACAGAC |
| RNF185 | CTGAGAACTCCAGTGCAGGG |
| RNF7 | CGATACGTGCGCCATCTGCA |
| RNF7 | AACAAACAAGAGGACTGTGT |
| RNF7 | CCTCAAGAAGTGGAACGCGG |
| RNF7 | AGCTCAGGCTCCAAGTCGGG |
| RPRD1B | GTGGATGAGCCAAAGGGACA |
| RPRD1B | GTTTACCTTTGCGGAGCTCG |
| RPRD1B | GGGTCAAGACCTACCAAGAG |
| RPRDIB | CAAGAACGAAGTGTGTATGG |
| RPS6KL1 | GGAGCAGATTCGCAACAGGG |
| RPS6KL1 | CGGCTGACCATCATCCCACA |
| RPS6KL1 | AACCCAAGTGAGCCCCCGAG |
| RPS6KL1 | AAATTACCAAATACCTGCGG |
| SEL1L3 | GTGTGCCTTGAGTGGAACAT |
| SEL1L3 | CTTGGGTATAAACACTACCA |
| SEL1L3 | GCATAGTCATAGATTAACGC |
| SEL1L3 | TTCCCGTGTACAAAAAAAGG |
| SEPW1 | CATCACTTCAAAGAACCCGG |
| SEPW1 | GGAGTTCCCTCGCCGCACTG |
| SEPW1 | GGGCTTACCAATAAACGACT |
| SEPW1 | CGCTTGAGGCTACAAGTCCA |
| SH3BGRL | CCTTACCGCTGTAGAGCCAG |
| SH3BGRL | TTGCAGCCAATGAAGAGAAT |
| SH3BGRL | AAGAAACAACAAGATGTGCT |
| SH3BGRL | GAAAATAGTCGACCAGCCAC |
| SIT1 | TGGCTGCACACTTGTCCCAG |
| SIT1 | TGCATTATCTACAGACAGGT |
| SIT1 | AGGAATCCCCTCCATAACCC |
| SIT1 | GAGCAGAGGCGACAACTGCA |
| SLC47A1 | TTCATAAGCTCCGTGTTCTG |
| SLC47A1 | GAGGTCGGGAGCTTCCTCAG |
| SLC47A1 | AAACTGCATCAAGCTACATG |
| SLC47A1 | GCAACTCCAGTTACGATCTG |
| SLC9A3 | GAAGAACACTACGATGATGG |
| SLC9A3 | CTGTCCGGATATGTCCTCGA |
| SLC9A3 | ACTCACCCATGAGCCCACTG |
| SLC9A3 | GCTGAACGACGCAGTCACCG |
| SP1 | CATCATCCGGACACCAACAG |
| SP1 | GTATGTGACCAATGTACCAG |
| SP1 | CAACAGATTATCACAAATCG |
| SP1 | TTACTACCAGTGGATCATCA |
| ST5 | AGCATGGGAAGGTCGCCGAG |
| ST5 | CTGTGCCTCTTGGGTAATCG |
| ST5 | CGCCCCCAGCTATCGCACGC |
| ST5 | GAAGGAGGTAGACCATTACT |
| STAT6 | GCTGGAAGGCCTCCATACTG |
| STAT6 | CAGCCACCACAAAGGGCACG |
| STAT6 | ATCAAGCGGTGTGAGCGGAA |
| STAT6 | GCTGGGAATAAATGTCCACC |
| TBL1Y | GGGACAGGGACTCTATAGGG |
| TBL1Y | AGGCTAGCAAATCACTGACA |
| TBL1Y | GACATTGTATACGAGAAGGG |
| TBL1Y | AAATCCTCCAAAGAACCGAG |
| TICRR | GACGCTGCTAGACTACCAGT |
| TICRR | GGATAGAGTCAATATACCTG |
| TICRR | TCAAAGAGATCACTAAAGCG |
| TMEM62 | CAGGAATCCGGTCAATAATG |
| TMEM62 | ATGAACGAATAGTTGCCAAA |
| TMEM62 | GAGACTGGAAGGATAATAGG |
| TMEM62 | GTAGAATGGCAAACCTACCA |
| TNK1 | TGGGCCTAAGTCTAAGAACT |
| TNK1 | CCCAAACATCCACACGTCCG |
| TNK1 | AATGGTCGCACCTTCAAAGT |
| TNK1 | CCTCTGGGATCAGACACTTG |
| TNS2 | GCAGCTGTAGTCAGGCATCG |
| TNS2 | GCTGAGCTACGAGATCCCTA |
| TNS2 | GTAACATGTTATCACAAGGG |
| TNS2 | GGTCGTACTATACTGCAAGG |
| TP53BP1 | TCATGTGACGATGTAAGACA |
| TP53BP1 | GAAGCCCATTAGTCCTGTCA |
| TP53BP1 | GGAATCAATACTAATCACAC |
| TP53BP1 | AGACCCATGATCCCATACTT |
| TTN | AGAACCTGCAACAATCACCG |
| TTN | GTACCTAACGACGAAAGGTG |
| TTN | GTCCTTGTAGGATAGCAATG |
| TTN | AGAGGTTCAATAAAGTACGG |
| TUFM | ACAGGCACTGCACCCCTCGA |
| TUFM | AGCAGAGCCTACGATGACTG |
| TUFM | TCGGGGTATCACCATCAATG |
| TUFM | GTGACAGGTACACTAGAGCG |
| UPK1B | GCCCACAAATATGCCGATCC |
| UPK1B | TGCTGACAGGACAATTGCTG |
| UPK1B | TTGCGGCATTGCCCTGACTG |
| UPK1B | CTCCAAACAATGATGACCAG |
| UQCRC1 | AAGGTAGAGCATCATCACGG |
| UQCRC1 | ATGTCCATGGGATGCCACCG |
| UQCRC1 | CACCGTGCAAGTGGGCTGAG |
| UQCRC1 | GGGCCTTGTAATGTGTGCTG |
| WWOX | CAAGGTAGAAGCAATGACCC |
| WWOX | ACACCGAGGAGAAGACTCAG |
| WWOX | CCAAGATCACATGTGCACCA |
| WWOX | GCCGTCGTATCTTTGCCGGG |
| XCL1 | GAGACTTCACTCCCTACACC |
| XCL1 | TGCTGATCCACAAGCCACAT |
| XCL1 | TCTGCTAACCGGCAGTCGCT |
| XCL1 | GGAGTGAAGTCTCAGATAAG |
| ZBTB7A | ACCGTCAGCACAGCCAACGT |
| ZBTB7A | GAGTCGCGGGCCGACGACAA |
| ZBTB7A | GCCGTAGTGGCCGTTCTGCG |
| ZBTB7A | ATCATCGGACGCCCCAAAGG |
| ZFYVE28 | CCCCCACAAGCTTAGCACTG |
| ZFYVE28 | ATGTGTTTGCAGCTACGTCT |
| ZFYVE28 | CTTGAGCAACAACAATCTCG |
| ZFYVE28 | TTGCTGCGGAAAATAAGGTG |
| ZNF101 | CATGAAAGAACTCATAGTGG |
| ZNF101 | CCCGTAAACAGAAACAACAT |
| ZNF101 | TGAAATCAGATCTCACGCGC |
| ZNF101 | AATAAACTGGGATAATCGAT |
| ZNF436 | CTCACCTAATCCAACACCAA |
| ZNF436 | GATAGGTCAGAAAGACAATG |
| ZNF436 | TGAGATCAGGAGTGAGAACG |
| ZNF436 | AATGAGAACATATATGATAG |
| ZNF506 | AAAAACCTTTAACTATGAAG |
| ZNF506 | GAGTGTCCAGTGCACAAAAG |
| ZNF506 | TATGTGAAAAAAGGGTTGCA |
| ZNF716 | CATTTCAAATGTAAAAACGA |
| ZNF716 | CAAGACCTTCAGTCAGAGCA |
| ZNF716 | GGTTAGTAAGTGTTGAAGAG |
| ZNF716 | TGTAGTTAGTAAGTGTTGAG |
| ZNF805 | GTCCATGACTGTGACTCACA |
| ZNF805 | GCCACATTCGAAGCACTCGT |
| ZNF805 | AAAAGAGATTCCTTCAGACA |
| ZNF805 | AGGGAGAACGCTTGAGACCA |

TABLE 2

| Name | Sequence | Type |
|---|---|---|
| ADORA2A_g1 | ATGCTAGGTTGGAACAACTG | CGS |
| ADORA2A_g2 | CGAGGAGCCCAUGAUGGGCA | CGS |
| FAM105A_g1 | GAAGTGACCAAGTTCACTCC | CGS |
| FAM105A_g2 | CUAGCAUCCAGGAGUGAACU | CGS |
| NKX2-6_g1 | TTTAGAGCCCGGCCTGAACG | CGS |
| NKX2-6_g2 | GUCCUUGACCGAGAAGGGGG | CGS |
| PDE4C_g1 | CTAGAAGACACCAACAAGTG | CGS |
| PDE4C_g2 | CUCUCCUUUCAGCUUUGACC | CGS |
| PLXNA4_g1 | GCGGTATGGACTCTCCACGC | CGS |
| PLXNA4_g2 | GCAGGUCCAGUUCCAGGGCA | CGS |
| AAVS1_g1 | GGGCCACTAGGGACAGGAT | CTRL |
| AAVS1_g2 | GTCACCAATCCTGTCCCTAG | CTRL |
| MOB3C_g1 | AGTGTGGTGAGGCTACCACC | CYC |
| MOB3C_g2 | CUGCUUCAGGCACAGGGCCA | CYC |
| NFKB2_g1 | GGGACCAGCCAAGATCGAGG | CYC |
| NFKB2_g2 | UCCCUCGUAGUUACAGAUCU | CYC |
| NFKBIA_g1 | GGTTGGTGATCACAGCCAAG | CYC |
| NFKBIA_g2 | CAUGGAUGAUGGCCAAGUGC | CYC |
| PIM2_g1 | CAAGCAGGCGGATCACGCCA | CYC |
| PIM2_g2 | CUCCGCCUGCGUAGGAGGCA | CYC |
| PPIA_g1 | CGCCGCCCGCCCGACCTCAA | CYC |
| PPIA_g2 | GUACCCUUACCACUCAGUCU | CYC |
| CBLB_g1 | TGCACAGAACTATCGTACCA | PAN |
| CBLB_g2 | AAAUAUCAAGUAUAUAUGGU | PAN |
| CYC1_g1 | GGTGGGCGTGTGCTACACGG | PAN |
| CYC1_g2 | AGCGGAGAUCUUGCAGGCAG | PAN |
| FAM49B_g1 | TATGAGGATTAACAATGTAC | PAN |
| FAM49B_g2 | CUUCUCAGACUCUGUAGGCU | PAN |
| RASA2_g1 | AGATATCACACATTACAGTG P | AN |
| RASA2_g2 | AUUUUGUGGGGUCCAAGAUA | PAN |
| TMEM222_g1 | ACGGACATGAAGCAATATCA | PAN |
| TMEM222_g2 | UGUCACAGCAGAGAUUGUGC | PAN |
| FKBP1A_g1 | UUCACAGGGAUGCUUGAAGA | TAC |
| FKBP1A_g2 | CUGGGAAGAAGGGGUUGCCC | TAC |
| PFN1_g1 | GATCTTCGTACCAAGAGCAC | PAN |
| PFN1_g2 | GUUCCUCUUCCAGCCAGCUG | PAN |
| TGIF2_g1 | GGAGTCGGTGAAGATCCTCC | TAC |
| TGIF2_g2 | CUAGCCCCUAGGCACCAUGU | TAC |
| GTF2I_g1 | CAACATGAGACTGGAAAAGA | TGFB |
| GTF2I_g2 | UGUAUUGUAGUGUAAAGAAC | TGFB |
| LHX8_g1 | GACACCGGACATGCCAGCAT | TGFB |
| LHX8_g2 | UUCCUGCAGGUGAGCCCCGA | TGFB |
| SIRT7_g1 | GCGTCTATCCCAGACTACCG | TGFB |
| SIRT7_g2 | CGCAGGUGUCGCGCAUCCUG | TGFB |
| TGFBR1_g1 | TAAAAGGGCGATCTAATGAA | TGFB |
| TGFBR1_g2 | UGGCAGAAACACUGUAACGC | TGFB |
| TGFBR2_g1 | GCAGAAGCTGAGTTCAACCT | TGFB |
| TGFBR2 g2 | UAUCAUGUCGUUAUUAACUG | TGFB |

1. A genetically modified hematopoietic cell that comprises a genetic modification to a T-cell negative regulator gene that inhibits expression or activity of the polypeptide product encoded by the T-cell negative regulator gene in one or more immunosuppressive conditions in a tumor microenvironment (TME), wherein expression or activity of the polypeptide product is inhibited by at least 60% compared to a control wild-type hematopoietic cell.

2. The genetically modified hematopoietic cell of claim 1, wherein the genetic modification to the T-cell negative regulator gene inactivates the gene.

3. The genetically modified hematopoietic cell of claim 1, wherein the genetically modified hematopoietic cells is a T-cell.

4. The genetically modified hematopoietic cell of claim 3, wherein the T-cell is a CD8+ T cell or CD4+ T cell.

5. The genetically modified hematopoietic cell of claim 1, wherein the T-cell negative regulator gene is inhibited using a clustered, regularly interspaced, short palindromic repeats (CRISPR) system: a TALEN system, a zinc finger nuclease system, a meganuclease system, an siRNA, an antisense RNA, microRNA, or hairpin RNA.

6.-10. (canceled)

11. The genetically modified-T hematopoietic cell of claim 1, wherein the T-cell negative regulator gene is selected from the group consisting of ALAS1, AMBRA1, ANKRD32, ARHGAP15, C15orf40, C3orf33, C8orf44, CARKD, CD300LB, CENPB, CHL1, CHST3, CLEC4M, COL15A1, COL25A1, CORO1A, CUL3, CWC27, CYC1, DOK2, DUSP4, ENG, FAM49B, FKBPIA, FUBP1, GAB3, GLRX, GREB1L, GTF2H2, GTF2I, HAUS1, HISTH2AD, HISTIH2BC, HOXA10, IGFBP4, IRF2BP2, IYD, KCNK4, KDM6B, LICAM, LAMA3, LHX8, MOB3C, MBTD1, MRPL17, MRPL33, MTIF2, MYOIH, NEFL, NFκB1A, NFκB2, NMT1, ORC6, PCBP2, PCGF1, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNF185, RNF7, RPRD1B, RPS6KL1, SELIL3, SEPW1, SH3BGRL, SIRT7, SIT1, SLC47A1, SLC9A3, SP1, ST5, STAT6, TBL1Y, TGFBR1, TGFBR2, TGIF2, TICRR, TMEM62, TNK1, TNS2, TP53BP1, TTN, TUFM, UPK1B, UQCRC1, WWOX XCL1, ZBTB7A, ZFYVE28, ZNF01, ZNF436, ZNF506, ZNF716, and ZNF805.

12.-16. (canceled)

17. The genetically modified hematopoietic cell of claim 1, wherein the T-cell negative regulator gene is CENPB, CD300LB, IYD, ST5, RNF7, MBTD1, MRPL33, MYOIH, PIWIL4, ZNF805, HISTIH2BC, UPK1B, LAMA3, ENG, ORC6, TICRR, C15orf40, TUFM, RNF185, PTPRG, HAUS1, TMEM62, IGFBP4, LICAM, or MTIF2.

18. The genetically modified-4 hematopoietic cell of claim 1, wherein the T-cell negative regulator gene is PFN1, FAM49B, PDE4C, NKX2-6, FKBP1A, GTF2I, LHX8, MOB3C, NFκB1A, PIM2, PLXNA4, FAM105A, SIRT7, or TGIF2.

19. A population of cell comprising the genetically modified hematopoietic cell of claim 1.

20. A method of treating cancer comprising administering a population of cells comprising a genetically modified hematopoietic cell ofany claim 1 to a subject that has cancer.

21. A genetically modified T cell that has modulated immune function compared to a control wildtype T cell and comprises a genetic modification to inhibit expression of a polypeptide encoded by a T cell gene, wherein expression of the polypeptide is inhibited by at least 60% compared to the control wild-type T cell; and the gene is selected from the group consisting of ALAS1, AMBRA1, ANKRD32, ARHGAP15, C15orf40, C3orf33, C8orf44, CARKD, CD300LB, CENPB, CHL1, CHST3, CLEC4M, COL15A1, COL25A1, CORO1A, CUL3, CWC27, CYC1, DOK2, DUSP4, ENG, FAM49B, FKBP1A, FUBP1, GAB3, GLRX, GREB1L, GTF2H2, GTF2I, HAUS1, HIST1H2AD, HISTIH2BC, HOXA10, IGFBP4, IRF2BP2, IYD, KCNK4, KDM6B, LICAM, LAMA3, LHX8, MOB3C, MBTD1, MRPL17, MRPL33, MTIF2, MYOIH, NEFL, NFKB1A, NFκB2, NMT1, ORC6, PCBP2, PCGF1, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNF185, RNF7, RPRD1B, RPS6KL1, SELIL3, SEPW1, SH3BGRL, SIRT7, SIT1, SLC47A1, SLC9A3, SP1, ST5, STAT6, TBLIY, TGFBR1, TGFBR2, TGIF2, TICRR, TMEM62, TNK1, TNS2, TP53BP1, TTN, TUFM, UPK1B, UQCRC1, WWOX XCL1, ZBTB7A, ZFYVE28, ZNF01, ZNF436, ZNF506, ZNF716, and ZNF805.

22. The genetically modified T cell of claim 21, wherein the gene is inactivated.

23. The genetically modified T cell of claim 22, wherein the T cell is a CD8+ or CD4+ T cell.

24. The genetically modified T cell of claim 21, wherein the gene is inhibited using a CRISPR system, a TALEN system, a zinc finger nuclease system, a meganuclease system, an siRNA, an antisense RNA, microRNA, or a hairpin RNA.

25. A cell culture comprising a genetically modified T cell of claim 21.

26. A method of generating a genetically modified cell population for treatment of a subject that has cancer, the method comprising:
obtaining hematopoietic cells from the patient;
inhibiting expression of a T-cell inhibitory gene selected from the group consisting of ALAS1, AMBRA1, ANKRD32, ARHGAP15, C15orf40, C3orf33, C8orf44, CARKD, CD300LB, CENPB, CHL1, CHST3, CLEC4M, COL15A1, COL25A1, CORO1A, CUL3, CWC27, CYC1, DOK2, DUSP4, ENG, FAM49B, FKBP1A, FUBP1, GAB3, GLRX GREB1L, GTF2H2, GTF2I, HAUS1, HIST1H2AD, HISTIH2BC, HOXA10, IGFBP4, IRF2BP2, IYD, KCNK4, KDM6B, LICAM, LAMA3, LHX8, MOB3C, MBTD1, MRPL17, MRPL33, MTIF2, MYOIH, NEFL, NFκB1A, NFκB2, NMT1, ORC6, PCBP2, PCGF1, PDCD6IP, PDCL, PFN1, PIM2, PIWIL4, PLGLB2, PLXNA4, PDE4C, NXX2-6, POTEJ, PPIA, PPP2R5D, PTPRG, RFPL1, RNF13, RNFI85, RNF7, RPRD1B, RPS6KL1, SELIL3, SEPW1, SH3BGRL, SIRT7, SIT1, SLC47A1, SLC9A3, SP1, ST5, STAT6, TBL1Y, TGFBR1, TGFBR2, TGIF2, TICRR, TMEM62, TNK1, TNS2, TP53BP1, TTN, TUFM, UPK1B, UQCRC1, WWOX XCL1, ZBTB7A, ZFYVE28, ZNF01, ZNF436, ZNF506, ZNF716, and ZNF805;
selecting hematopoietic cells in which the T-cell inhibitory gene is inhibited.
expanding the selected hematopoietic cell population ex vivo.

27. The method of claim 26, wherein the hematopoietic cells are hematopoietic stem cells.

28. The method of claim 26, wherein the hematopoietic cells are T cells.

29. The method of claim 26, wherein the hematopoietic cells are CD8+ or CD4+ T cells.

30. The method of claim 26, wherein the T-cell inhibitory gene is inhibited using a CRISPR system, a TALEN system, a zinc finger nuclease system, a meganuclease system, an siRNA, an antisense RNA, microRNA, or a short hairpin RNA.


SEQUENCE LISTING (406 sequences)

SEQ ID NO: 1 - DNA, 20 bp, synthetic construct
  misc_feature 1..20: note = Gene: ALAS1
  misc_feature 1..20: note = Description of Sequence: sgRNA
  source 1..20: mol_type = other DNA
tggtgcagtaatgactacct

SEQ ID NO: 2 - DNA, 20 bp, synthetic construct
  misc_feature 1..20: note = Gene: ALAS1
  misc_feature 1..20: note = Description of Sequence: sgRNA
  source 1..20: mol_type = other DNA
tcgaatcccttggatcatgg

SEQ ID NO: 3 - DNA, 20 bp, synthetic construct
  misc_feature 1..20: note = Gene: ALAS1
  misc_feature 1..20: note = Description of Sequence: sgRNA
  source 1..20: mol_type = other DNA
acagatcaaagaaacccctc

SEQ ID NO: 4 - DNA, 20 bp, synthetic construct
  misc_feature 1..20: note = Gene: ALAS1
  misc_feature 1..20: note = Description of Sequence: sgRNA
  source 1..20: mol_type = other DNA
ccacaatcttggggactgag

SEQ ID NO: 5 - DNA, 20 bp, synthetic construct
  misc_feature 1..20: note = Gene: AMBRA1
  misc_feature 1..20: note = Description of Sequence: sgRNA
  source 1..20: mol_type = other DNA
ggagctggggacctgccaag

SEQ ID NO: 6 - DNA, 20 bp, synthetic construct
  misc_feature 1..20: note = Gene: AMBRA1
  misc_feature 1..20: note = Description of Sequence: sgRNA
  source 1..20: mol_type = other DNA
tttctcggtgatacctagaa

SEQ ID NO: 7 - DNA, 20 bp, synthetic construct
  misc_feature 1..20: note = Gene: AMBRA1
  misc_feature 1..20: note = Description of Sequence: sgRNA
  source 1..20: mol_type = other DNA
ccataatatctatattacgg

SEQ ID NO: 8 - DNA, 20 bp, synthetic construct
  misc_feature 1..20: note = Gene: AMBRA1
  misc_feature 1..20: note = Description of Sequence: sgRNA
  source 1..20: mol_type = other DNA
ggatgatccagtatctctca

SEQ ID NO: 9 - DNA, 20 bp, synthetic construct
  misc_feature 1..20: note = Gene: ANKRD32
  misc_feature 1..20: note = Description of Sequence: sgRNA
  source 1..20: mol_type = other DNA
gcttatcagttctaacaagg

SEQ ID NO: 10 - DNA, 20 bp, synthetic construct
  misc_feature 1..20: note = Gene: ANKRD32
  misc_feature 1..20: note = Description of Sequence: sgRNA
  source 1..20: mol_type = other DNA
gcatgaagaacgcatacagg

SEQ ID NO: 11 - DNA, 20 bp, synthetic construct
  misc_feature 1..20: note = Gene: ANKRD32
  misc_feature 1..20: note = Description of Sequence: sgRNA
  source 1..20: mol_type = other DNA
catggctattaagacagatg

SEQ ID NO: 12 - DNA, 20 bp, synthetic construct
  misc_feature 1..20: note = Gene: ANKRD32
  misc_feature 1..20: note = Description of Sequence: sgRNA
  source 1..20: mol_type = other DNA
agagaccatgtatagaaccc

SEQ ID NO: 13 - DNA, 20 bp, synthetic construct
  misc_feature 1..20: note = Gene: ARHGAP15
  misc_feature 1..20: note = Description of Sequence: sgRNA
  source 1..20: mol_type = other DNA
atgcattgctttacaaacca

SEQ ID NO: 14 - DNA, 20 bp, synthetic construct
  misc_feature 1..20: note = Gene: ARHGAP15
  misc_feature 1..20: note = Description of Sequence: sgRNA
  source 1..20: mol_type = other DNA
ccagaaagtgtggatttgtg

SEQ ID NO: 15 - DNA, 20 bp, synthetic construct
  misc_feature 1..20: note = Gene: ARHGAP15
  misc_feature 1..20: note = Description of Sequence: sgRNA
  source 1..20: mol_type = other DNA
ttcagtgaccttcccgacat

SEQ ID NO: 16 - DNA, 20 bp, synthetic construct
  misc_feature 1..20: note = Gene: ARHGAP15
  misc_feature 1..20: note = Description of Sequence: sgRNA
  source 1..20: mol_type = other DNA
acttagcaattcagtgctag

SEQ ID NO: 17 - DNA, 20 bp, synthetic construct
  misc_feature 1..20: note = Gene: C15orf40
  misc_feature 1..20: note = Description of Sequence: sgRNA
  source 1..20: mol_type = other DNA
cgggcaacacccaatactcg

SEQ ID NO: 18 - DNA, 20 bp, synthetic construct
  misc_feature 1..20: note = Gene: C15orf40
  misc_feature 1..20: note = Description of Sequence: sgRNA
  source 1..20: mol_type = other DNA
cctgtggcagttgatcctaa

SEQ ID NO: 19 - DNA, 20 bp, synthetic construct
  misc_feature 1..20: note = Gene: C15orf40
  misc_feature 1..20: note = Description of Sequence: sgRNA
  source 1..20: mol_type = other DNA
atagccatccatgcaaaacc

SEQ ID NO: 20 - DNA, 20 bp, synthetic construct
  misc_feature 1..20: note = Gene: C15orf40
  misc_feature 1..20: note = Description of Sequence: sgRNA
  source 1..20: mol_type = other DNA
gaaggctggtgcgacgacca

SEQ ID NO: 21 - DNA, 20 bp, synthetic construct
  misc_feature 1..20: note = Gene: C3orf33
  misc_feature 1..20: note = Description of Sequence: sgRNA
  source 1..20: mol_type = other DNA
ttaaccagcaaagcaccacg

SEQ ID NO: 22 - DNA, 20 bp, synthetic construct
  misc_feature 1..20: note = Gene: C3orf33
  misc_feature 1..20: note = Description of Sequence: sgRNA
  source 1..20: mol_type = other DNA
tggaaccatagtaattggga

SEQ ID NO: 23 - DNA, 20 bp, synthetic construct
  misc_feature 1..20: note = Gene: C3orf33
  misc_feature 1..20: note = Description of Sequence: sgRNA
  source 1..20: mol_type = other DNA
aatatgattctaaaatctac

SEQ ID NO: 24 - DNA, 20 bp, synthetic construct
  misc_feature 1..20: note = Gene: C3orf33
  misc_feature 1..20: note = Description of Sequence: sgRNA
  source 1..20: mol_type = other DNA
agaagaaatgttaaactacg

SEQ ID NO: 25 - DNA, 20 bp, synthetic construct
  misc_feature 1..20: note = Gene: C8orf44
  misc_feature 1..20: note = Description of Sequence: sgRNA
  source 1..20: mol_type = other DNA
gggagcacttcgaaaaccac

SEQ ID NO: 26 - DNA, 20 bp, synthetic construct
  misc_feature 1..20: note = Gene: C8orf44
  misc_feature 1..20: note = Description of Sequence: sgRNA
  source 1..20: mol_type = other DNA
cctcccatgagggaaagtgt

SEQ ID NO: 27 - DNA, 20 bp, synthetic construct
  misc_feature 1..20: note = Gene: C8orf44
  misc_feature 1..20: note = Description of Sequence: sgRNA
  source 1..20: mol_type = other DNA
agaagcagaaaaggcaaaag

SEQ ID NO: 28 - DNA, 20 bp, synthetic construct
  misc_feature 1..20: note = Gene: CARKD
  misc_feature 1..20: note = Description of Sequence: sgRNA
  source 1..20: mol_type = other DNA
catccctgttgtcatcgacg

SEQ ID NO: 29 - DNA, 20 bp, synthetic construct
  misc_feature 1..20: note = Gene: CARKD
  misc_feature 1..20: note = Description of Sequence: sgRNA
  source 1..20: mol_type = other DNA
cctgtgttgtcatagctcag

SEQ ID NO: 30 - DNA, 20 bp, synthetic construct
  misc_feature 1..20: note = Gene: CARKD
  misc_feature 1..20: note = Description of Sequence: sgRNA
  source 1..20: mol_type = other DNA
ggaagaataggcgtagttgg

SEQ ID NO: 31 - DNA, 20 bp, synthetic construct
  misc_feature 1..20: note = Gene: CARKD
  misc_feature 1..20: note = Description of Sequence: sgRNA
  source 1..20: mol_type = other DNA
cgcactggcacagaacacgt

SEQ ID NO: 32 - DNA, 20 bp, synthetic construct
  misc_feature 1..20: note = Gene: CD300LB
  misc_feature 1..20: note = Description of Sequence: sgRNA
  source 1..20: mol_type = other DNA
acgcagatgtttactggtgt

SEQ ID NO: 33 - DNA, 20 bp, synthetic construct
  misc_feature 1..20: note = Gene: CD300LB
  misc_feature 1..20: note = Description of Sequence: sgRNA
  source 1..20: mol_type = other DNA
gggagacctacattaagtgg

SEQ ID NO: 34 - DNA, 20 bp, synthetic construct
  misc_feature 1..20: note = Gene: CD300LB
  misc_feature 1..20: note = Description of Sequence: sgRNA
  source 1..20: mol_type = other DNA
cctcattgaaaccagagggt

SEQ ID NO: 35 - DNA, 20 bp, synthetic construct
  misc_feature 1..20: note = Gene: CD300LB
  misc_feature 1..20: note = Description of Sequence: sgRNA
  source 1..20: mol_type = other DNA
atagtggcattgaaccgtca

SEQ ID NO: 36 - DNA, 20 bp, synthetic construct
  misc_feature 1..20: note = Gene: CENPB
  misc_feature 1..20: note = Description of Sequence: sgRNA
  source 1..20: mol_type = other DNA
gtaccatagactggtctcgg

SEQ ID NO: 37 - DNA, 20 bp, synthetic construct
  misc_feature 1..20: note = Gene: CENPB
  misc_feature 1..20: note = Description of Sequence: sgRNA
  source 1..20: mol_type = other DNA
catagccgcctgctttcgtg

SEQ ID NO: 38 - DNA, 20 bp, synthetic construct
  misc_feature 1..20: note = Gene: CENPB
  misc_feature 1..20: note = Description of Sequence: sgRNA
  source 1..20: mol_type = other DNA
gggatcctggccctctagcg

SEQ ID NO: 39 - DNA, 20 bp, synthetic construct
  misc_feature 1..20: note = Gene: CHL1
  misc_feature 1..20: note = Description of Sequence: sgRNA
  source 1..20: mol_type = other DNA
tttaaacttactgtcaactg

SEQ ID NO: 40 - DNA, 20 bp, synthetic construct
  misc_feature 1..20: note = Gene: CHL1
  misc_feature 1..20: note = Description of Sequence: sgRNA
  source 1..20: mol_type = other DNA
cattcaggatcccaaacgag

SEQ ID NO: 41 - DNA, 20 bp, synthetic construct
  misc_feature 1..20: note = Gene: CHL1
  misc_feature 1..20: note = Description of Sequence: sgRNA
  source 1..20: mol_type = other DNA
tggagaaaattacgctacag

SEQ ID NO: 42 - DNA, 20 bp, synthetic construct
  misc_feature 1..20: note = Gene: CHL1
  misc_feature 1..20: note = Description of Sequence: sgRNA
  source 1..20: mol_type = other DNA
agatctatacttcgcaaacg

SEQ ID NO: 43 - DNA, 20 bp, synthetic construct
  misc_feature 1..20: note = Gene: CHST3
  misc_feature 1..20: note = Description of Sequence: sgRNA
  source 1..20: mol_type = other DNA
cactgcaagaaccgccgctg

SEQ ID NO: 44 - DNA, 20 bp, synthetic construct
  misc_feature 1..20: note = Gene: CHST3
  misc_feature 1..20: note = Description of Sequence: sgRNA
  source 1..20: mol_type = other DNA
actcagttcatgttccgccg

SEQ ID NO: 45 - DNA, 20 bp, synthetic construct
  misc_feature 1..20: note = Gene: CHST3
  misc_feature 1..20: note = Description of Sequence: sgRNA
  source 1..20: mol_type = other DNA
gaagaactcgcccacgaacg

SEQ ID NO: 46 - DNA, 20 bp, synthetic construct
  misc_feature 1..20: note = Gene: CHST3
  misc_feature 1..20: note = Description of Sequence: sgRNA
  source 1..20: mol_type = other DNA
gagcacgtcgcggtacacca

SEQ ID NO: 47 - DNA, 20 bp, synthetic construct
  misc_feature 1..20: note = Gene: CLEC4M
  misc_feature 1..20: note = Description of Sequence: sgRNA
  source 1..20: mol_type = other DNA
gaagaagatccaacaaccag

SEQ ID NO: 48 - DNA, 20 bp, synthetic construct
  misc_feature 1..20: note = Gene: CLEC4M
  misc_feature 1..20: note = Description of Sequence: sgRNA
  source 1..20: mol_type = other DNA
gaccccagccaagagcatga

SEQ ID NO: 49 - DNA, 20 bp, synthetic construct
  misc_feature 1..20: note = Gene: CLEC4M
  misc_feature 1..20: note = Description of Sequence: sgRNA
  source 1..20: mol_type = other DNA
ggagatctaccaggagctga

SEQ ID NO: 50 - DNA, 20 bp, synthetic construct
  misc_feature 1..20: note = Gene: CLEC4M
  misc_feature 1..20: note = Description of Sequence: sgRNA
  source 1..20: mol_type = other DNA
tccagtccttgggacagtgg

SEQ ID NO: 51 - DNA, 20 bp, synthetic construct
  misc_feature 1..20: note = Gene: COL15A1
  misc_feature 1..20: note = Description of Sequence: sgRNA
  source 1..20: mol_type = other DNA
ctgtacgatccttaccacag

SEQ ID NO: 52 - DNA, 20 bp, synthetic construct
  misc_feature 1..20: note = Gene: COL15A1
  misc_feature 1..20: note = Description of Sequence: sgRNA
  source 1..20: mol_type = other DNA
ctcctcacagttcacgagga

SEQ ID NO: 53 - DNA, 20 bp, synthetic construct
  misc_feature 1..20: note = Gene: COL15A1
  misc_feature 1..20: note = Description of Sequence: sgRNA
  source 1..20: mol_type = other DNA
tggcgaagagcacgccacca

SEQ ID NO: 54 - DNA, 20 bp, synthetic construct
  misc_feature 1..20: note = Gene: COL15A1
  misc_feature 1..20: note = Description of Sequence: sgRNA
  source 1..20: mol_type = other DNA
aaagttccatgtcttcaaag

SEQ ID NO: 55 - DNA, 20 bp, synthetic construct
  misc_feature 1..20: note = Gene: COL25A1
  misc_feature 1..20: note = Description of Sequence: sgRNA
  source 1..20: mol_type = other DNA
taagcactaccttaatcagg

SEQ ID NO: 56 - DNA, 20 bp, synthetic construct
  misc_feature 1..20: note = Gene: COL25A1
  misc_feature 1..20: note = Description of Sequence: sgRNA
  source 1..20: mol_type = other DNA
aaaccaacgacctccaggcg

SEQ ID NO: 57 - DNA, 20 bp, synthetic construct
  misc_feature 1..20: note = Gene: COL25A1
  misc_feature 1..20: note = Description of Sequence: sgRNA
  source 1..20: mol_type = other DNA
gggtccacctggtcaaaaag

SEQ ID NO: 58 - DNA, 20 bp, synthetic construct
  misc_feature 1..20: note = Gene: COL25A1
  misc_feature 1..20: note = Description of Sequence: sgRNA
  source 1..20: mol_type = other DNA
atttgtgacagggtgaacgg

SEQ ID NO: 59 - DNA, 20 bp, synthetic construct
  misc_feature 1..20: note = Gene: CORO1A
  misc_feature 1..20: note = Description of Sequence: sgRNA
  source 1..20: mol_type = other DNA
gtgcagtgttcgtgtcggag

SEQ ID NO: 60 - DNA, 20 bp, synthetic construct
  misc_feature 1..20: note = Gene: CORO1A
  misc_feature 1..20: note = Description of Sequence: sgRNA
  source 1..20: mol_type = other DNA
cccagacacgatctacagtg

SEQ ID NO: 61 - DNA, 20 bp, synthetic construct
  misc_feature 1..20: note = Gene: CORO1A
  misc_feature 1..20: note = Description of Sequence: sgRNA
  source 1..20: mol_type = other DNA
gccacagaggtagacgatgt

SEQ ID NO: 62 - DNA, 20 bp, synthetic construct
  misc_feature 1..20: note = Gene: CORO1A
  misc_feature 1..20: note = Description of Sequence: sgRNA
  source 1..20: mol_type = other DNA
gtgcctttagactggacgtg

SEQ ID NO: 63 - DNA, 20 bp, synthetic construct
  misc_feature 1..20: note = Gene: CUL3
  misc_feature 1..20: note = Description of Sequence: sgRNA
  source 1..20: mol_type = other DNA
atccagcgtaagaataacag

SEQ ID NO: 64 - DNA, 20 bp, synthetic construct
  misc_feature 1..20: note = Gene: CUL3
  misc_feature 1..20: note = Description of Sequence: sgRNA
  source 1..20: mol_type = other DNA
ggtgtattagggatcatcta

SEQ ID NO: 65 - DNA, 20 bp, synthetic construct
  misc_feature 1..20: note = Gene: CUL3
  misc_feature 1..20: note = Description of Sequence: sgRNA
  source 1..20: mol_type = other DNA
ataacttgtacatgcaacca

SEQ ID NO: 66 - DNA, 20 bp, synthetic construct
  misc_feature 1..20: note = Gene: CUL3
  misc_feature 1..20: note = Description of Sequence: sgRNA
  source 1..20: mol_type = other DNA
gagcatctcaaacacaacga

SEQ ID NO: 67 - DNA, 20 bp, synthetic construct
  misc_feature 1..20: note = Gene: CWC27
  misc_feature 1..20: note = Description of Sequence: sgRNA
  source 1..20: mol_type = other DNA
cctggtttcatagtccaagg

SEQ ID NO: 68 - DNA, 20 bp, synthetic construct
  misc_feature 1..20: note = Gene: CWC27
  misc_feature 1..20: note = Description of Sequence: sgRNA
  source 1..20: mol_type = other DNA
actggaacagaactgagatg

SEQ ID NO: 69 - DNA, 20 bp, synthetic construct
  misc_feature 1..20: note = Gene: CWC27
  misc_feature 1..20: note = Description of Sequence: sgRNA
  source 1..20: mol_type = other DNA
tttaatttgataggttacag

SEQ ID NO: 70 - DNA, 20 bp, synthetic construct
  misc_feature 1..20: note = Gene: CWC27
  misc_feature 1..20: note = Description of Sequence: sgRNA
  source 1..20: mol_type = other DNA
gagatattgacatagagttg

SEQ ID NO: 71 - DNA, 20 bp, synthetic construct
  misc_feature 1..20: note = Gene: CYC1
  misc_feature 1..20: note = Description of Sequence: sgRNA
  source 1..20: mol_type = other DNA
ggtgggcgtgtgctacacgg

SEQ ID NO: 72 - DNA, 20 bp, synthetic construct
  misc_feature 1..20: note = Gene: CYC1
  misc_feature 1..20: note = Description of Sequence: sgRNA
  source 1..20: mol_type = other DNA
agctatccgtggtctcaccg

SEQ ID NO: 73 - DNA, 20 bp, synthetic construct
  misc_feature 1..20: note = Gene: CYC1
  misc_feature 1..20: note = Description of Sequence: sgRNA
  source 1..20: mol_type = other DNA
aaggccagacttcgacgaca

SEQ ID NO: 74 - DNA, 20 bp, synthetic construct
  misc_feature 1..20: note = Gene: CYC1
  misc_feature 1..20: note = Description of Sequence: sgRNA
  source 1..20: mol_type = other DNA
aaaaccataccccaacagtg

SEQ ID NO: 75 - DNA, 20 bp, synthetic construct
  misc_feature 1..20: note = Gene: DOK2
  misc_feature 1..20: note = Description of Sequence: sgRNA
  source 1..20: mol_type = other DNA
gtagggccagtcgtacagct

SEQ ID NO: 76 - DNA, 20 bp, synthetic construct
  misc_feature 1..20: note = Gene: DOK2
  misc_feature 1..20: note = Description of Sequence: sgRNA
  source 1..20: mol_type = other DNA
ccccgcgcgactcacgggga

SEQ ID NO: 77 - DNA, 20 bp, synthetic construct
  misc_feature 1..20: note = Gene: DOK2
  misc_feature 1..20: note = Description of Sequence: sgRNA
  source 1..20: mol_type = other DNA
gtccgaccctccatacagtg

SEQ ID NO: 78 - DNA, 20 bp, synthetic construct
  misc_feature 1..20: note = Gene: DOK2
  misc_feature 1..20: note = Description of Sequence: sgRNA
  source 1..20: mol_type = other DNA
ggagggtataggacccccgc

SEQ ID NO: 79 - DNA, 20 bp, synthetic construct
  misc_feature 1..20: note = Gene: DUSP4
  misc_feature 1..20: note = Description of Sequence: sgRNA
  source 1..20: mol_type = other DNA
agcgcacgttgaccgaacct

SEQ ID NO: 80 - DNA, 20 bp, synthetic construct
  misc_feature 1..20: note = Gene: DUSP4
  misc_feature 1..20: note = Description of Sequence: sgRNA
  source 1..20: mol_type = other DNA
gcgctccggcctctactcgg

SEQ ID NO: 81 - DNA, 20 bp, synthetic construct
  misc_feature 1..20: note = Gene: DUSP4
  misc_feature 1..20: note = Description of Sequence: sgRNA
  source 1..20: mol_type = other DNA
tcagtacaagtgcatcccag

SEQ ID NO: 82 - DNA, 20 bp, synthetic construct
  misc_feature 1..20: note = Gene: DUSP4
  misc_feature 1..20: note = Description of Sequence: sgRNA
  source 1..20: mol_type = other DNA
tgggaccccactacacgacc

SEQ ID NO: 83 - DNA, 20 bp, synthetic construct
  misc_feature 1..20: note = Gene: ENG
  misc_feature 1..20: note = Description of Sequence: sgRNA
  source 1..20: mol_type = other DNA
atgagccaggacacgtaggg

SEQ ID NO: 84 - DNA, 20 bp, synthetic construct
  misc_feature 1..20: note = Gene: ENG
  misc_feature 1..20: note = Description of Sequence: sgRNA
  source 1..20: mol_type = other DNA
cgtactccagccttggtccg

SEQ ID NO: 85 - DNA, 20 bp, synthetic construct
  misc_feature 1..20: note = Gene: ENG
  misc_feature 1..20: note = Description of Sequence: sgRNA
  source 1..20: mol_type = other DNA
aggagtggtctggatcggtg

SEQ ID NO: 86 - DNA, 20 bp, synthetic construct
  misc_feature 1..20: note = Gene: ENG
  misc_feature 1..20: note = Description of Sequence: sgRNA
  source 1..20: mol_type = other DNA
accactagccaggtctcgaa

SEQ ID NO: 87 - DNA, 20 bp, synthetic construct
  misc_feature 1..20: note = Gene: FAM49B
  misc_feature 1..20: note = Description of Sequence: sgRNA
  source 1..20: mol_type = other DNA
agctggccacgaaatacgag

SEQ ID NO: 88 - DNA, 20 bp, synthetic construct
  misc_feature 1..20: note = Gene: FAM49B
  misc_feature 1..20: note = Description of Sequence: sgRNA
  source 1..20: mol_type = other DNA
tatgaggattaacaatgtac

SEQ ID NO: 89 - DNA, 20 bp, synthetic construct
  misc_feature 1..20: note = Gene: FAM49B
  misc_feature 1..20: note = Description of Sequence: sgRNA
  source 1..20: mol_type = other DNA
ccagcctacagagtctgaga

SEQ ID NO: 90 - DNA, 20 bp, synthetic construct
  misc_feature 1..20: note = Gene: FUBP1
  misc_feature 1..20: note = Description of Sequence: sgRNA
  source 1..20: mol_type = other DNA
caaaaattggaggtgatgca

SEQ ID NO: 91 - DNA, 20 bp, synthetic construct
  misc_feature 1..20: note = Gene: FUBP1
  misc_feature 1..20: note = Description of Sequence: sgRNA
  source 1..20: mol_type = other DNA
tgattgtaacaggaacgggc

SEQ ID NO: 92 - DNA, 20 bp, synthetic construct
  misc_feature 1..20: note = Gene: FUBP1
  misc_feature 1..20: note = Description of Sequence: sgRNA
  source 1..20: mol_type = other DNA
atgatgggacaacacccgaa

SEQ ID NO: 93 - DNA, 20 bp, synthetic construct
  misc_feature 1..20: note = Gene: FUBP1
  misc_feature 1..20: note = Description of Sequence: sgRNA
  source 1..20: mol_type = other DNA
gagaagttcggaatgagtat

SEQ ID NO: 94 - DNA, 20 bp, synthetic construct
  misc_feature 1..20: note = Gene: GAB3
  misc_feature 1..20: note = Description of Sequence: sgRNA
  source 1..20: mol_type = other DNA
tcatgttgtctaaaccagag

SEQ ID NO: 95 - DNA, 20 bp, synthetic construct
  misc_feature 1..20: note = Gene: GAB3
  misc_feature 1..20: note = Description of Sequence: sgRNA
  source 1..20: mol_type = other DNA
ttcccgtacattctacctgg

SEQ ID NO: 96 - DNA, 20 bp, synthetic construct
  misc_feature 1..20: note = Gene: GAB3
  misc_feature 1..20: note = Description of Sequence: sgRNA
  source 1..20: mol_type = other DNA
acctcagcgagtgtgcagtg

SEQ ID NO: 97 - DNA, 20 bp, synthetic construct
  misc_feature 1..20: note = Gene: GAB3
  misc_feature 1..20: note = Description of Sequence: sgRNA
  source 1..20: mol_type = other DNA
gaccagctatcacatctggt

SEQ ID NO: 98 - DNA, 20 bp, synthetic construct
  misc_feature 1..20: note = Gene: GLRX
  misc_feature 1..20: note = Description of Sequence: sgRNA
  source 1..20: mol_type = other DNA
aatctcgttagtgtggttgg

SEQ ID NO: 99 - DNA, 20 bp, synthetic construct
  misc_feature 1..20: note = Gene: GLRX
  misc_feature 1..20: note = Description of Sequence: sgRNA
  source 1..20: mol_type = other DNA
cacctgcccgtactgcagga

SEQ ID NO: 100 - DNA, 20 bp, synthetic construct
  misc_feature 1..20: note = Gene: GLRX
  misc_feature 1..20: note = Description of Sequence: sgRNA
  source 1..20: mol_type = other DNA
agattatttgcaacagctca

SEQ ID NO: 101 - DNA, 20 bp, synthetic construct
  misc_feature 1..20: note = Gene: GLRX
  misc_feature 1..20: note = Description of Sequence: sgRNA
  source 1..20: mol_type = other DNA
gtcaattgcccatcaaacaa

SEQ ID NO: 102 - DNA, 20 bp, synthetic construct
  misc_feature 1..20: note = Gene: GREB1L
  misc_feature 1..20: note = Description of Sequence: sgRNA
  source 1..20: mol_type = other DNA
gtcgcggagtgacgccatgg

SEQ ID NO: 103 - DNA, 20 bp, synthetic construct
  misc_feature 1..20: note = Gene: GREB1L
  misc_feature 1..20: note = Description of Sequence: sgRNA
  source 1..20: mol_type = other DNA
agcttgagtcaattaaccgg

SEQ ID NO: 104 - DNA, 20 bp, synthetic construct
  misc_feature 1..20: note = Gene: GREB1L
  misc_feature 1..20: note = Description of Sequence: sgRNA
  source 1..20: mol_type = other DNA
actcccatgtggaactaacg

SEQ ID NO: 105 - DNA, 20 bp, synthetic construct
  misc_feature 1..20: note = Gene: GREB1L
  misc_feature 1..20: note = Description of Sequence: sgRNA
  source 1..20: mol_type = other DNA
ttagctgcggttgataaatg

SEQ ID NO: 106 - DNA, 20 bp, synthetic construct
  misc_feature 1..20: note = Gene: GTF2H2
  misc_feature 1..20: note = Description of Sequence: sgRNA
  source 1..20: mol_type = other DNA
attagatcataaatattaga

SEQ ID NO: 107 - DNA, 20 bp, synthetic construct
  misc_feature 1..20: note = Gene: GTF2H2
  misc_feature 1..20: note = Description of Sequence: sgRNA
  source 1..20: mol_type = other DNA
aacgtctttgaagaaagctg

SEQ ID NO: 108 - DNA, 20 bp, synthetic construct
  misc_feature 1..20: note = Gene: GTF2H2
  misc_feature 1..20: note = Description of Sequence: sgRNA
  source 1..20: mol_type = other DNA
catctactaccacataaagg

SEQ ID NO: 109 - DNA, 20 bp, synthetic construct
  misc_feature 1..20: note = Gene: GTF2H2
  misc_feature 1..20: note = Description of Sequence: sgRNA
  source 1..20: mol_type = other DNA
ggctttcatctaaaataaca

SEQ ID NO: 110 - DNA, 20 bp, synthetic construct
  misc_feature 1..20: note = Gene: HAUS1
  misc_feature 1..20: note = Description of Sequence: sgRNA
  source 1..20: mol_type = other DNA
taagaattacctagctagcg

SEQ ID NO: 111 - DNA, 20 bp, synthetic construct
  misc_feature 1..20: note = Gene: HAUS1
  misc_feature 1..20: note = Description of Sequence: sgRNA
  source 1..20: mol_type = other DNA
tgataatcgtcgtcagaaca

SEQ ID NO: 112 - DNA, 20 bp, synthetic construct
  misc_feature 1..20: note = Gene: HAUS1
  misc_feature 1..20: note = Description of Sequence: sgRNA
  source 1..20: mol_type = other DNA
cagtgctagagagattggcg

SEQ ID NO: 113 - DNA, 20 bp, synthetic construct
  misc_feature 1..20: note = Gene: HAUS1
  misc_feature 1..20: note = Description of Sequence: sgRNA
  source 1..20: mol_type = other DNA
gcaagtgaatacgagtcaga

SEQ ID NO: 114 - DNA, 20 bp, synthetic construct
  misc_feature 1..20: note = Gene: HIST1H2AD
  misc_feature 1..20: note = Description of Sequence: sgRNA
  source 1..20: mol_type = other DNA
ggagtccggcccgcgaagag

SEQ ID NO: 115 - DNA, 20 bp, synthetic construct
  misc_feature 1..20: note = Gene: HIST1H2AD
  misc_feature 1..20: note = Description of Sequence: sgRNA
  source 1..20: mol_type = other DNA
acgcggcaagcaaggcggaa

SEQ ID NO: 116 - DNA, 20 bp, synthetic construct
  misc_feature 1..20: note = Gene: HIST1H2AD
  misc_feature 1..20: note = Description of Sequence: sgRNA
  source 1..20: mol_type = other DNA
gctcggagtagttgcccttg

SEQ ID NO: 117 - DNA, 20 bp, synthetic construct
  misc_feature 1..20: note = Gene: HIST1H2AD
  misc_feature 1..20: note = Description of Sequence: sgRNA
  source 1..20: mol_type = other DNA
caactactccgagcgagtcg

SEQ ID NO: 118 - DNA, 20 bp, synthetic construct
  misc_feature 1..20: note = Gene: HIST1H2BC
  misc_feature 1..20: note = Description of Sequence: sgRNA
  source 1..20: mol_type = other DNA
cgacatatttgagcgcatcg

SEQ ID NO: 119 - DNA, 20 bp, synthetic construct
  misc_feature 1..20: note = Gene: HIST1H2BC
  misc_feature 1..20: note = Description of Sequence: sgRNA
  source 1..20: mol_type = other DNA
gaaagaattcatgatgccca

SEQ ID NO: 120 - DNA, 20 bp, synthetic construct
  misc_feature 1..20: note = Gene: HIST1H2BC
  misc_feature 1..20: note = Description of Sequence: sgRNA
  source 1..20: mol_type = other DNA
cttggaagagatgccagtgt

SEQ ID NO: 121 - DNA, 20 bp, synthetic construct
  misc_feature 1..20: note = Gene: HIST1H2BC
  misc_feature 1..20: note = Description of Sequence: sgRNA
  source 1..20: mol_type = other DNA
acacagagtaactctccttg

SEQ ID NO: 122 - DNA, 20 bp, synthetic construct
  misc_feature 1..20: note = Gene: HOXA10
  misc_feature 1..20: note = Description of Sequence: sgRNA
  source 1..20: mol_type = other DNA
ctactgcctctacgactcgg

SEQ ID NO: 123 - DNA, 20 bp, synthetic construct
  misc_feature 1..20: note = Gene: HOXA10
  misc_feature 1..20: note = Description of Sequence: sgRNA
  source 1..20: mol_type = other DNA
gaccaaaaaagagttcgcgg

SEQ ID NO: 124 - DNA, 20 bp, synthetic construct
  misc_feature 1..20: note = Gene: HOXA10
  misc_feature 1..20: note = Description of Sequence: sgRNA
  source 1..20: mol_type = other DNA
ggcggttactacgcccacgg

SEQ ID NO: 125 - DNA, 20 bp, synthetic construct
  misc_feature 1..20: note = Gene: HOXA10
  misc_feature 1..20: note = Description of Sequence: sgRNA
  source 1..20: mol_type = other DNA
agatcgaaaccgcgccccgg

SEQ ID NO: 126 - DNA, 20 bp, synthetic construct
  misc_feature 1..20: note = Gene: IGFBP4
  misc_feature 1..20: note = Description of Sequence: sgRNA
  source 1..20: mol_type = other DNA
ctgaatacagacaaggacga

SEQ ID NO: 127 - DNA, 20 bp, synthetic construct
  misc_feature 1..20: note = Gene: IGFBP4
  misc_feature 1..20: note = Description of Sequence: sgRNA
  source 1..20: mol_type = other DNA
cacacactgatgcacgggca

SEQ ID NO: 128 - DNA, 20 bp, synthetic construct
  misc_feature 1..20: note = Gene: IGFBP4
  misc_feature 1..20: note = Description of Sequence: sgRNA
  source 1..20: mol_type = other DNA
acaggccgggcatcctcccg

SEQ ID NO: 129 - DNA, 20 bp, synthetic construct
  misc_feature 1..20: note = Gene: IRF2BP2
  misc_feature 1..20: note = Description of Sequence: sgRNA
  source 1..20: mol_type = other DNA
gccccctaagatcaacggag

SEQ ID NO: 130 - DNA, 20 bp, synthetic construct
  misc_feature 1..20: note = Gene: IRF2BP2
  misc_feature 1..20: note = Description of Sequence: sgRNA
  source 1..20: mol_type = other DNA
ttcaccgaacccgtctgccg

SEQ ID NO: 131 - DNA, 20 bp, synthetic construct
  misc_feature 1..20: note = Gene: IRF2BP2
  misc_feature 1..20: note = Description of Sequence: sgRNA
  source 1..20: mol_type = other DNA
caacggcttctccaagctag

SEQ ID NO: 132 - DNA, 20 bp, synthetic construct
  misc_feature 1..20: note = Gene: IRF2BP2
  misc_feature 1..20: note = Description of Sequence: sgRNA
  source 1..20: mol_type = other DNA
ggccgacagcctgtccaccg

SEQ ID NO: 133 - DNA, 20 bp, synthetic construct
  misc_feature 1..20: note = Gene: IYD
  misc_feature 1..20: note = Description of Sequence: sgRNA
  source 1..20: mol_type = other DNA
tcacagacctcaagaaactg

SEQ ID NO: 134 - DNA, 20 bp, synthetic construct
  misc_feature 1..20: note = Gene: IYD
  misc_feature 1..20: note = Description of Sequence: sgRNA
  source 1..20: mol_type = other DNA
tcataaccactatcctgaga

SEQ ID NO: 135 - DNA, 20 bp, synthetic construct
  misc_feature 1..20: note = Gene: IYD
  misc_feature 1..20: note = Description of Sequence: sgRNA
  source 1..20: mol_type = other DNA
tgggtccttcacaaccacga

SEQ ID NO: 136 - DNA, 20 bp, synthetic construct
  misc_feature 1..20: note = Gene: IYD
  misc_feature 1..20: note = Description of Sequence: sgRNA
  source 1..20: mol_type = other DNA
caagattcgaaagatcattg

SEQ ID NO: 137 - DNA, 20 bp, synthetic construct
  misc_feature 1..20: note = Gene: KCNK4
  misc_feature 1..20: note = Description of Sequence: sgRNA
  source 1..20: mol_type = other DNA
gacagcactcttactagctc

SEQ ID NO: 138 - DNA, 20 bp, synthetic construct
  misc_feature 1..20: note = Gene: KCNK4
  misc_feature 1..20: note = Description of Sequence: sgRNA
  source 1..20: mol_type = other DNA
atagtgacgcttaccaccgt

SEQ ID NO: 139 - DNA, 20 bp, synthetic construct
  misc_feature 1..20: note = Gene: KCNK4
  misc_feature 1..20: note = Description of Sequence: sgRNA
  source 1..20: mol_type = other DNA
gccagtaggatcccaaacag

SEQ ID NO: 140 - DNA, 20 bp, synthetic construct
  misc_feature 1..20: note = Gene: KCNK4
  misc_feature 1..20: note = Description of Sequence: sgRNA
  source 1..20: mol_type = other DNA
atatagcagaacacgaacgt

SEQ ID NO: 141 - DNA, 20 bp, synthetic construct
  misc_feature 1..20: note = Gene: KDM6B
  misc_feature 1..20: note = Description of Sequence: sgRNA
  source 1..20: mol_type = other DNA
gcagtcggaaaccgttcttg

SEQ ID NO: 142 - DNA, 20 bp, synthetic construct
  misc_feature 1..20: note = Gene: KDM6B
  misc_feature 1..20: note = Description of Sequence: sgRNA
  source 1..20: mol_type = other DNA
gctggacgaatccattcgca

SEQ ID NO: 143 - DNA, 20 bp, synthetic construct
  misc_feature 1..20: note = Gene: KDM6B
  misc_feature 1..20: note = Description of Sequence: sgRNA
  source 1..20: mol_type = other DNA
ggtgctagaagagatcagcc

SEQ ID NO: 144 - DNA, 20 bp, synthetic construct
  misc_feature 1..20: note = Gene: KDM6B
  misc_feature 1..20: note = Description of Sequence: sgRNA
  source 1..20: mol_type = other DNA
gacaaaagtactgttatcgg

SEQ ID NO: 145 - DNA, 20 bp, synthetic construct
  misc_feature 1..20: note = Gene: L1CAM
  misc_feature 1..20: note = Description of Sequence: sgRNA
  source 1..20: mol_type = other DNA
gagtagccgatagtgacctg

SEQ ID NO: 146 - DNA, 20 bp, synthetic construct
  misc_feature 1..20: note = Gene: L1CAM
  misc_feature 1..20: note = Description of Sequence: sgRNA
  source 1..20: mol_type = other DNA
gcatgcgtactatgtcaccg

SEQ ID NO: 147 - DNA, 20 bp, synthetic construct
  misc_feature 1..20: note = Gene: L1CAM
  misc_feature 1..20: note = Description of Sequence: sgRNA
  source 1..20: mol_type = other DNA
cacaatggtgacccaatgtg

SEQ ID NO: 148 - DNA, 20 bp, synthetic construct
  misc_feature 1..20: note = Gene: L1CAM
  misc_feature 1..20: note = Description of Sequence: sgRNA
  source 1..20: mol_type = other DNA
cttggggacagtgacaagtg

SEQ ID NO: 149 - DNA, 20 bp, synthetic construct
  misc_feature 1..20: note = Gene: LAMA3
  misc_feature 1..20: note = Description of Sequence: sgRNA
  source 1..20: mol_type = other DNA
gagatccaactgtcactcgg

SEQ ID NO: 150 - DNA, 20 bp, synthetic construct
  misc_feature 1..20: note = Gene: LAMA3
  misc_feature 1..20: note = Description of Sequence: sgRNA
  source 1..20: mol_type = other DNA
ctgacaagagtcaccagcgt

SEQ ID NO: 151 - DNA, 20 bp, synthetic construct
  misc_feature 1..20: note = Gene: LAMA3
  misc_feature 1..20: note = Description of Sequence: sgRNA
  source 1..20: mol_type = other DNA
tgtggtcgagtattccacgg

SEQ ID NO: 152 - DNA, 20 bp, synthetic construct
  misc_feature 1..20: note = Gene: LAMA3
  misc_feature 1..20: note = Description of Sequence: sgRNA
  source 1..20: mol_type = other DNA
gagcggcaggtatcacatcg

SEQ ID NO: 153 - DNA, 20 bp, synthetic construct
  misc_feature 1..20: note = Gene: MBTD1
  misc_feature 1..20: note = Description of Sequence: sgRNA
  source 1..20: mol_type = other DNA
ttatgtgacaggcacctatg

SEQ ID NO: 154 - DNA, 20 bp, synthetic construct
  misc_feature 1..20: note = Gene: MBTD1
  misc_feature 1..20: note = Description of Sequence: sgRNA
  source 1..20: mol_type = other DNA
attggttggtctcgaagcat

SEQ ID NO: 155 - DNA, 20 bp, synthetic construct
  misc_feature 1..20: note = Gene: MBTD1
  misc_feature 1..20: note = Description of Sequence: sgRNA
  source 1..20: mol_type = other DNA
gtgtcgaacacgagtagcag

SEQ ID NO: 156 - DNA, 20 bp, synthetic construct
  misc_feature 1..20: note = Gene: MBTD1
  misc_feature 1..20: note = Description of Sequence: sgRNA
  source 1..20: mol_type = other DNA
agcttgatactgagcatatg

SEQ ID NO: 157 - DNA, 20 bp, synthetic construct
  misc_feature 1..20: note = Gene: MRPL17
  misc_feature 1..20: note = Description of Sequence: sgRNA
  source 1..20: mol_type = other DNA
tcggtacaaagatcaaactg

SEQ ID NO: 158 - DNA, 20 bp, synthetic construct
  misc_feature 1..20: note = Gene: MRPL17
  misc_feature 1..20: note = Description of Sequence: sgRNA
  source 1..20: mol_type = other DNA
ggcccatacggcgaaatacg

SEQ ID NO: 159 - DNA, 20 bp, synthetic construct
  misc_feature 1..20: note = Gene: MRPL17
  misc_feature 1..20: note = Description of Sequence: sgRNA
  source 1..20: mol_type = other DNA
acgaacgcatcgaggcacca

SEQ ID NO: 160 - DNA, 20 bp, synthetic construct
  misc_feature 1..20: note = Gene: MRPL17
  misc_feature 1..20: note = Description of Sequence: sgRNA
  source 1..20: mol_type = other DNA
taacgaacgagccatgcgca

SEQ ID NO: 161 - DNA, 20 bp, synthetic construct
  misc_feature 1..20: note = Gene: MRPL33
  misc_feature 1..20: note = Description of Sequence: sgRNA
  source 1..20: mol_type = other DNA
tgagaatggtgagcgaagct

SEQ ID NO: 162 - DNA, 20 bp, synthetic construct
  misc_feature 1..20: note = Gene: MRPL33
  misc_feature 1..20: note = Description of Sequence: sgRNA
  source 1..20: mol_type = other DNA
gccccacttacagaagaccg

SEQ ID NO: 163 - DNA, 20 bp, synthetic construct
  misc_feature 1..20: note = Gene: MRPL33
  misc_feature 1..20: note = Description of Sequence: sgRNA
  source 1..20: mol_type = other DNA
acaccaagagaaaccgactg

SEQ ID NO: 164 - DNA, 20 bp, synthetic construct
  misc_feature 1..20: note = Gene: MRPL33
  misc_feature 1..20: note = Description of Sequence: sgRNA
  source 1..20: mol_type = other DNA
tagaaacattctggtgagaa

SEQ ID NO: 165 - DNA, 20 bp, synthetic construct
  misc_feature 1..20: note = Gene: MTIF2
  misc_feature 1..20: note = Description of Sequence: sgRNA
  source 1..20: mol_type = other DNA
atggattggaatgactattg

SEQ ID NO: 166 - DNA, 20 bp, synthetic construct
  misc_feature 1..20: note = Gene: MTIF2
  misc_feature 1..20: note = Description of Sequence: sgRNA
  source 1..20: mol_type = other DNA
gatcaaagaagtgataacga

SEQ ID NO: 167 - DNA, 20 bp, synthetic construct
  misc_feature 1..20: note = Gene: MTIF2
  misc_feature 1..20: note = Description of Sequence: sgRNA
  source 1..20: mol_type = other DNA
ccccgcatttaccgtaagtg

SEQ ID NO: 168 - DNA, 20 bp, synthetic construct
  misc_feature 1..20: note = Gene: MTIF2
  misc_feature 1..20: note = Description of Sequence: sgRNA
  source 1..20: mol_type = other DNA
gcccattatagtaacaactg

SEQ ID NO: 169 - DNA, 20 bp, synthetic construct
  misc_feature 1..20: note = Gene: MYO1H
  misc_feature 1..20: note = Description of Sequence: sgRNA
  source 1..20: mol_type = other DNA
tccagcaactgttaattgag

SEQ ID NO: 170 - DNA, 20 bp, synthetic construct
  misc_feature 1..20: note = Gene: MYO1H
  misc_feature 1..20: note = Description of Sequence: sgRNA
  source 1..20: mol_type = other DNA
agaggtatttatacagctgg

SEQ ID NO: 171 - DNA, 20 bp, synthetic construct
  misc_feature 1..20: note = Gene: MYO1H
  misc_feature 1..20: note = Description of Sequence: sgRNA
  source 1..20: mol_type = other DNA
tggccgagttagaaaaccgg

SEQ ID NO: 172 - DNA, 20 bp, synthetic construct
  misc_feature 1..20: note = Gene: MYO1H
  misc_feature 1..20: note = Description of Sequence: sgRNA
  source 1..20: mol_type = other DNA
gtacatcggctacaaacccg

SEQ ID NO: 173 - DNA, 20 bp, synthetic construct
  misc_feature 1..20: note = Gene: NEFL
  misc_feature 1..20: note = Description of Sequence: sgRNA
  source 1..20: mol_type = other DNA
caagaacatgcagaacgctg

SEQ ID NO: 174 - DNA, 20 bp, synthetic construct
  misc_feature 1..20: note = Gene: NEFL
  misc_feature 1..20: note = Description of Sequence: sgRNA
  source 1..20: mol_type = other DNA
tcttggccttgagcagacga

SEQ ID NO: 175 - DNA, 20 bp, synthetic construct
  misc_feature 1..20: note = Gene: NEFL
  misc_feature 1..20: note = Description of Sequence: sgRNA
  source 1..20: mol_type = other DNA
cctgcgtgcggatggacttg

SEQ ID NO: 176 - DNA, 20 bp, synthetic construct
  misc_feature 1..20: note = Gene: NEFL
  misc_feature 1..20: note = Description of Sequence: sgRNA
  source 1..20: mol_type = other DNA
agcgcgcaaaggcgccgacg

SEQ ID NO: 177 - DNA, 20 bp, synthetic construct
  misc_feature 1..20: note = Gene: NFKB2
  misc_feature 1..20: note = Description of Sequence: sgRNA
  source 1..20: mol_type = other DNA
gggaccagccaagatcgagg

SEQ ID NO: 178 - DNA, 20 bp, synthetic construct
  misc_feature 1..20: note = Gene: NFKB2
  misc_feature 1..20: note = Description of Sequence: sgRNA
  source 1..20: mol_type = other DNA
ctgcaactgaaacgcaagcg

SEQ ID NO: 179 - DNA, 20 bp, synthetic construct
  misc_feature 1..20: note = Gene: NFKB2
  misc_feature 1..20: note = Description of Sequence: sgRNA
  source 1..20: mol_type = other DNA
cccactccatagaatctccg

SEQ ID NO: 180 - DNA, 20 bp, synthetic construct
  misc_feature 1..20: note = Gene: NFKB2
  misc_feature 1..20: note = Description of Sequence: sgRNA
  source 1..20: mol_type = other DNA
actcgactacggcgtcaccg

SEQ ID NO: 181 - DNA, 20 bp, synthetic construct
  misc_feature 1..20: note = Gene: NMT1
  misc_feature 1..20: note = Description of Sequence: sgRNA
  source 1..20: mol_type = other DNA
aggacaacagctacaaccgg

SEQ ID NO: 182 - DNA, 20 bp, synthetic construct
  misc_feature 1..20: note = Gene: NMT1
  misc_feature 1..20: note = Description of Sequence: sgRNA
  source 1..20: mol_type = other DNA
gcatgtacatacccagcttg

SEQ ID NO: 183 - DNA, 20 bp, synthetic construct
  misc_feature 1..20: note = Gene: NMT1
  misc_feature 1..20: note = Description of Sequence: sgRNA
  source 1..20: mol_type = other DNA
gggttcgagtggtctcaagt

SEQ ID NO: 184 - DNA, 20 bp, synthetic construct
  misc_feature 1..20: note = Gene: NMT1
  misc_feature 1..20: note = Description of Sequence: sgRNA
  source 1..20: mol_type = other DNA
gggctttggtagtaccaccc

SEQ ID NO: 185 - DNA, 20 bp, synthetic construct
  misc_feature 1..20: note = Gene: ORC6
  misc_feature 1..20: note = Description of Sequence: sgRNA
  source 1..20: mol_type = other DNA
gacctacttaccctgtccaa

SEQ ID NO: 186 - DNA, 20 bp, synthetic construct
  misc_feature 1..20: note = Gene: ORC6
  misc_feature 1..20: note = Description of Sequence: sgRNA
  source 1..20: mol_type = other DNA
tcttccccagacacagcaag

SEQ ID NO: 187 - DNA, 20 bp, synthetic construct
  misc_feature 1..20: note = Gene: ORC6
  misc_feature 1..20: note = Description of Sequence: sgRNA
  source 1..20: mol_type = other DNA
gaggccgacacacttcaccc

SEQ ID NO: 188 - DNA, 20 bp, synthetic construct
  misc_feature 1..20: note = Gene: ORC6
  misc_feature 1..20: note = Description of Sequence: sgRNA
  source 1..20: mol_type = other DNA
tacagctaaactgtacagct

SEQ ID NO: 189 - DNA, 20 bp, synthetic construct
  misc_feature 1..20: note = Gene: PCBP2
  misc_feature 1..20: note = Description of Sequence: sgRNA
  source 1..20: mol_type = other DNA
gtgtgtcaaacagatctgcg

SEQ ID NO: 190 - DNA, 20 bp, synthetic construct
  misc_feature 1..20: note = Gene: PCBP2
  misc_feature 1..20: note = Description of Sequence: sgRNA
  source 1..20: mol_type = other DNA
caagatcaaggaaatacgag

SEQ ID NO: 191 - DNA, 20 bp, synthetic construct
  misc_feature 1..20: note = Gene: PCBP2
  misc_feature 1..20: note = Description of Sequence: sgRNA
  source 1..20: mol_type = other DNA
agttggcagtatcatcggaa

SEQ ID NO: 192 - DNA, 20 bp, synthetic construct
  misc_feature 1..20: note = Gene: PCBP2
  misc_feature 1..20: note = Description of Sequence: sgRNA
  source 1..20: mol_type = other DNA
cacgtatcaacatctcagaa

SEQ ID NO: 193 - DNA, 20 bp, synthetic construct
  misc_feature 1..20: note = Gene: PCGF1
  misc_feature 1..20: note = Description of Sequence: sgRNA
  source 1..20: mol_type = other DNA
ccagtcccgaggtttggacc

SEQ ID NO: 194 - DNA, 20 bp, synthetic construct
  misc_feature 1..20: note = Gene: PCGF1
  misc_feature 1..20: note = Description of Sequence: sgRNA
  source 1..20: mol_type = other DNA
ccacgaagtagccggcgcat

SEQ ID NO: 195 - DNA, 20 bp, synthetic construct
  misc_feature 1..20: note = Gene: PCGF1
  misc_feature 1..20: note = Description of Sequence: sgRNA
  source 1..20: mol_type = other DNA
gctcatcatagcgatagtag

SEQ ID NO: 196 - DNA, 20 bp, synthetic construct
  misc_feature 1..20: note = Gene: PCGF1
  misc_feature 1..20: note = Description of Sequence: sgRNA
  source 1..20: mol_type = other DNA
ccttgcacctcgttccgtag

SEQ ID NO: 197 - DNA, 20 bp, synthetic construct
  misc_feature 1..20: note = Gene: PDCD6IP
  misc_feature 1..20: note = Description of Sequence: sgRNA
  source 1..20: mol_type = other DNA
cgtccgctggacaagcacga

SEQ ID NO: 198 - DNA, 20 bp, synthetic construct
  misc_feature 1..20: note = Gene: PDCD6IP
  misc_feature 1..20: note = Description of Sequence: sgRNA
  source 1..20: mol_type = other DNA
cacacttgtgaaatctaccc

SEQ ID NO: 199 - DNA, 20 bp, synthetic construct
  misc_feature 1..20: note = Gene: PDCD6IP
  misc_feature 1..20: note = Description of Sequence: sgRNA
  source 1..20: mol_type = other DNA
aatttaaggaacgttggcaa

SEQ ID NO: 200 - DNA, 20 bp, synthetic construct
  misc_feature 1..20: note = Gene: PDCD6IP
  misc_feature 1..20: note = Description of Sequence: sgRNA
  source 1..20: mol_type = other DNA
cttaagtcgagagccgaccg

SEQ ID NO: 201 - DNA, 20 bp, synthetic construct
  misc_feature 1..20: note = Gene: PDCL
  misc_feature 1..20: note = Description of Sequence: sgRNA
  source 1..20: mol_type = other DNA
gaaggcatctcagttaacac

SEQ ID NO: 202 - DNA, 20 bp, synthetic construct
  misc_feature 1..20: note = Gene: PDCL
  misc_feature 1..20: note = Description of Sequence: sgRNA
  source 1..20: mol_type = other DNA
gcagtaccggaagcagcgaa

SEQ ID NO: 203 - DNA, 20 bp, synthetic construct
  misc_feature 1..20: note = Gene: PDCL
  misc_feature 1..20: note = Description of Sequence: sgRNA
  source 1..20: mol_type = other DNA
gaccacgaggacaaggaccg

SEQ ID NO: 204 - DNA, 20 bp, synthetic construct
  misc_feature 1..20: note = Gene: PDCL
  misc_feature 1..20: note = Description of Sequence: sgRNA
  source 1..20: mol_type = other DNA
ggagtttgccataatgaatg

SEQ ID NO: 205 - DNA, 20 bp, synthetic construct
  misc_feature 1..20: note = Gene: PIWIL4
  misc_feature 1..20: note = Description of Sequence: sgRNA
  source 1..20: mol_type = other DNA
tgtccatgtaccaaattgga

SEQ ID NO: 206 - DNA, 20 bp, synthetic construct
  misc_feature 1..20: note = Gene: PIWIL4
  misc_feature 1..20: note = Description of Sequence: sgRNA
  source 1..20: mol_type = other DNA
aatctggaatatatgtcaca

SEQ ID NO: 207 - DNA, 20 bp, synthetic construct
  misc_feature 1..20: note = Gene: PIWIL4
  misc_feature 1..20: note = Description of Sequence: sgRNA
  source 1..20: mol_type = other DNA
gtcgctacataaaatcaacc

SEQ ID NO: 208 - DNA, 20 bp, synthetic construct
  misc_feature 1..20: note = Gene: PIWIL4
  misc_feature 1..20: note = Description of Sequence: sgRNA
  source 1..20: mol_type = other DNA
ctgcttgtagtaatccacat

SEQ ID NO: 209 - DNA, 20 bp, synthetic construct
  misc_feature 1..20: note = Gene: PLGLB2
  misc_feature 1..20: note = Description of Sequence: sgRNA
  source 1..20: mol_type = other DNA
actgaacagtgaaggcccct

SEQ ID NO: 210 - DNA, 20 bp, synthetic construct
  misc_feature 1..20: note = Gene: PLGLB2
  misc_feature 1..20: note = Description of Sequence: sgRNA
  source 1..20: mol_type = other DNA
ggtattcacatagtcatcca

SEQ ID NO: 211 - DNA, 20 bp, synthetic construct
  misc_feature 1..20: note = Gene: PLGLB2
  misc_feature 1..20: note = Description of Sequence: sgRNA
  source 1..20: mol_type = other DNA
atgtgcagcaaaatgtgaag

SEQ ID NO: 212 - DNA, 20 bp, synthetic construct
  misc_feature 1..20: note = Gene: PLGLB2
  misc_feature 1..20: note = Description of Sequence: sgRNA
  source 1..20: mol_type = other DNA
tgtcactaagaagcagctgg

SEQ ID NO: 213 - DNA, 20 bp, synthetic construct
  misc_feature 1..20: note = Gene: POTEJ
  misc_feature 1..20: note = Description of Sequence: sgRNA
  source 1..20: mol_type = other DNA
aagcacggaagtactcacgt

SEQ ID NO: 214 - DNA, 20 bp, synthetic construct
  misc_feature 1..20: note = Gene: POTEJ
  misc_feature 1..20: note = Description of Sequence: sgRNA
  source 1..20: mol_type = other DNA
gtggtatctcggctccacga

SEQ ID NO: 215 - DNA, 20 bp, synthetic construct
  misc_feature 1..20: note = Gene: POTEJ
  misc_feature 1..20: note = Description of Sequence: sgRNA
  source 1..20: mol_type = other DNA
gtagatagcgtagtgtagag

SEQ ID NO: 216 - DNA, 20 bp, synthetic construct
  misc_feature 1..20: note = Gene: POTEJ
  misc_feature 1..20: note = Description of Sequence: sgRNA
  source 1..20: mol_type = other DNA
gttgtcaatgacgagcacag

SEQ ID NO: 217 - DNA, 20 bp, synthetic construct
  misc_feature 1..20: note = Gene: PPP2R5D
  misc_feature 1..20: note = Description of Sequence: sgRNA
  source 1..20: mol_type = other DNA
aagaggttcactgaaaactg

SEQ ID NO: 218 - DNA, 20 bp, synthetic construct
  misc_feature 1..20: note = Gene: PPP2R5D
  misc_feature 1..20: note = Description of Sequence: sgRNA
  source 1..20: mol_type = other DNA
agggacttgaccttgtgaag

SEQ ID NO: 219 - DNA, 20 bp, synthetic construct
  misc_feature 1..20: note = Gene: PPP2R5D
  misc_feature 1..20: note = Description of Sequence: sgRNA
  source 1..20: mol_type = other DNA
ggctccgggcttatatccgt

SEQ ID NO: 220 - DNA, 20 bp, synthetic construct
  misc_feature 1..20: note = Gene: PPP2R5D
  misc_feature 1..20: note = Description of Sequence: sgRNA
  source 1..20: mol_type = other DNA
cagcaaaatcaagtactcag

SEQ ID NO: 221 - DNA, 20 bp, synthetic construct
  misc_feature 1..20: note = Gene: PTPRG
  misc_feature 1..20: note = Description of Sequence: sgRNA
  source 1..20: mol_type = other DNA
ggtcccttggaatattcgag

SEQ ID NO: 222 - DNA, 20 bp, synthetic construct
  misc_feature 1..20: note = Gene: PTPRG
  misc_feature 1..20: note = Description of Sequence: sgRNA
  source 1..20: mol_type = other DNA
tccactatttcgctacacgg

SEQ ID NO: 223 - DNA, 20 bp, synthetic construct
  misc_feature 1..20: note = Gene: PTPRG
  misc_feature 1..20: note = Description of Sequence: sgRNA
  source 1..20: mol_type = other DNA
ctttcattagggaccactcg

SEQ ID NO: 224 - DNA, 20 bp, synthetic construct
  misc_feature 1..20: note = Gene: PTPRG
  misc_feature 1..20: note = Description of Sequence: sgRNA
  source 1..20: mol_type = other DNA
taaaaagcctatgtcccgcg

SEQ ID NO: 225 - DNA, 20 bp, synthetic construct
  misc_feature 1..20: note = Gene: RFPL1
  misc_feature 1..20: note = Description of Sequence: sgRNA
  source 1..20: mol_type = other DNA
gccaggggaaaagtgcacaa

SEQ ID NO: 226 - DNA, 20 bp, synthetic construct
  misc_feature 1..20: note = Gene: RFPL1
  misc_feature 1..20: note = Description of Sequence: sgRNA
  source 1..20: mol_type = other DNA
tctgtgtgatgcacccactt

SEQ ID NO: 227 - DNA, 20 bp, synthetic construct
  misc_feature 1..20: note = Gene: RFPL1
  misc_feature 1..20: note = Description of Sequence: sgRNA
  source 1..20: mol_type = other DNA
gacagcgcatccacactcca

SEQ ID NO: 228 - DNA, 20 bp, synthetic construct
  misc_feature 1..20: note = Gene: RFPL1
  misc_feature 1..20: note = Description of Sequence: sgRNA
  source 1..20: mol_type = other DNA
aatctgttcaccgcaaaggg

SEQ ID NO: 229 - DNA, 20 bp, synthetic construct
  misc_feature 1..20: note = Gene: RNF13
  misc_feature 1..20: note = Description of Sequence: sgRNA
  source 1..20: mol_type = other DNA
tggaggcactatgggttcac

SEQ ID NO: 230 - DNA, 20 bp, synthetic construct
  misc_feature 1..20: note = Gene: RNF13
  misc_feature 1..20: note = Description of Sequence: sgRNA
  source 1..20: mol_type = other DNA
atacctgtacttactgtcgt

SEQ ID NO: 231 - DNA, 20 bp, synthetic construct
  misc_feature 1..20: note = Gene: RNF13
  misc_feature 1..20: note = Description of Sequence: sgRNA
  source 1..20: mol_type = other DNA
ggaattaggtagtattccaa

SEQ ID NO: 232 - DNA, 20 bp, synthetic construct
  misc_feature 1..20: note = Gene: RNF13
  misc_feature 1..20: note = Description of Sequence: sgRNA
  source 1..20: mol_type = other DNA
tataaccaaatcttgcaggg

SEQ ID NO: 233 - DNA, 20 bp, synthetic construct
  misc_feature 1..20: note = Gene: RNF185
  misc_feature 1..20: note = Description of Sequence: sgRNA
  source 1..20: mol_type = other DNA
catcttacctgatgtaaaca

SEQ ID NO: 234 - DNA, 20 bp, synthetic construct
  misc_feature 1..20: note = Gene: RNF185
  misc_feature 1..20: note = Description of Sequence: sgRNA
  source 1..20: mol_type = other DNA
gtggccacacaggctgatga

SEQ ID NO: 235 - DNA, 20 bp, synthetic construct
  misc_feature 1..20: note = Gene: RNF185
  misc_feature 1..20: note = Description of Sequence: sgRNA
  source 1..20: mol_type = other DNA
ggagaccagacctaacagac

SEQ ID NO: 236 - DNA, 20 bp, synthetic construct
  misc_feature 1..20: note = Gene: RNF185
  misc_feature 1..20: note = Description of Sequence: sgRNA
  source 1..20: mol_type = other DNA
ctgagaactccagtgcaggg

SEQ ID NO: 237 - DNA, 20 bp, synthetic construct
  misc_feature 1..20: note = Gene: RNF7
  misc_feature 1..20: note = Description of Sequence: sgRNA
  source 1..20: mol_type = other DNA
cgatacgtgcgccatctgca

SEQ ID NO: 238 - DNA, 20 bp, synthetic construct
  misc_feature 1..20: note = Gene: RNF7
  misc_feature 1..20: note = Description of Sequence: sgRNA
  source 1..20: mol_type = other DNA
aacaaacaagaggactgtgt

SEQ ID NO: 239 - DNA, 20 bp, synthetic construct
  misc_feature 1..20: note = Gene: RNF7
  misc_feature 1..20: note = Description of Sequence: sgRNA
  source 1..20: mol_type = other DNA
cctcaagaagtggaacgcgg

SEQ ID NO: 240 - DNA, 20 bp, synthetic construct
  misc_feature 1..20: note = Gene: RNF7
  misc_feature 1..20: note = Description of Sequence: sgRNA
  source 1..20: mol_type = other DNA
agctcaggctccaagtcggg

SEQ ID NO: 241 - DNA, 20 bp, synthetic construct
  misc_feature 1..20: note = Gene: RPRD1B
  misc_feature 1..20: note = Description of Sequence: sgRNA
  source 1..20: mol_type = other DNA
gtggatgagccaaagggaca

SEQ ID NO: 242 - DNA, 20 bp, synthetic construct
  misc_feature 1..20: note = Gene: RPRD1B
  misc_feature 1..20: note = Description of Sequence: sgRNA
  source 1..20: mol_type = other DNA
gtttacctttgcggagctcg

SEQ ID NO: 243 - DNA, 20 bp, synthetic construct
  misc_feature 1..20: note = Gene: RPRD1B
  misc_feature 1..20: note = Description of Sequence: sgRNA
  source 1..20: mol_type = other DNA
gggtcaagacctaccaagag

SEQ ID NO: 244 - DNA, 20 bp, synthetic construct
  misc_feature 1..20: note = Gene: RPRD1B
  misc_feature 1..20: note = Description of Sequence: sgRNA
  source 1..20: mol_type = other DNA
caagaacgaagtgtgtatgg

SEQ ID NO: 245 - DNA, 20 bp, synthetic construct
  misc_feature 1..20: note = Gene: RPS6KL1
  misc_feature 1..20: note = Description of Sequence: sgRNA
  source 1..20: mol_type = other DNA
ggagcagattcgcaacaggg

SEQ ID NO: 246 - DNA, 20 bp, synthetic construct
  misc_feature 1..20: note = Gene: RPS6KL1
  misc_feature 1..20: note = Description of Sequence: sgRNA
  source 1..20: mol_type = other DNA
cggctgaccatcatcccaca

SEQ ID NO: 247 - DNA, 20 bp, synthetic construct
  misc_feature 1..20: note = Gene: RPS6KL1
  misc_feature 1..20: note = Description of Sequence: sgRNA
  source 1..20: mol_type = other DNA
aacccaagtgagcccccgag

SEQ ID NO: 248 - DNA, 20 bp, synthetic construct
  misc_feature 1..20: note = Gene: RPS6KL1
  misc_feature 1..20: note = Description of Sequence: sgRNA
  source 1..20: mol_type = other DNA
aaattaccaaatacctgcgg

SEQ ID NO: 249 - DNA, 20 bp, synthetic construct
  misc_feature 1..20: note = Gene: SEL1L3
  misc_feature 1..20: note = Description of Sequence: sgRNA
  source 1..20: mol_type = other DNA
gtgtgccttgagtggaacat

SEQ ID NO: 250 - DNA, 20 bp, synthetic construct
  misc_feature 1..20: note = Gene: SEL1L3
  misc_feature 1..20: note = Description of Sequence: sgRNA
  source 1..20: mol_type = other DNA
cttgggtataaacactacca

SEQ ID NO: 251 - DNA, 20 bp, synthetic construct
  misc_feature 1..20: note = Gene: SEL1L3
  misc_feature 1..20: note = Description of Sequence: sgRNA
  source 1..20: mol_type = other DNA
gcatagtcatagattaacgc

SEQ ID NO: 252 - DNA, 20 bp, synthetic construct
  misc_feature 1..20: note = Gene: SEL1L3
  misc_feature 1..20: note = Description of Sequence: sgRNA
  source 1..20: mol_type = other DNA
ttcccgtgtacaaaaaaagg

SEQ ID NO: 253 - DNA, 20 bp, synthetic construct
  misc_feature 1..20: note = Gene: SEPW1
  misc_feature 1..20: note = Description of Sequence: sgRNA
  source 1..20: mol_type = other DNA
catcacttcaaagaacccgg

SEQ ID NO: 254 - DNA, 20 bp, synthetic construct
  misc_feature 1..20: note = Gene: SEPW1
  misc_feature 1..20: note = Description of Sequence: sgRNA
  source 1..20: mol_type = other DNA
ggagttccctcgccgcactg

SEQ ID NO: 255 - DNA, 20 bp, synthetic construct
  misc_feature 1..20: note = Gene: SEPW1
  misc_feature 1..20: note = Description of Sequence: sgRNA
  source 1..20: mol_type = other DNA
gggcttaccaataaacgact

SEQ ID NO: 256 - DNA, 20 bp, synthetic construct
  misc_feature 1..20: note = Gene: SEPW1
  misc_feature 1..20: note = Description of Sequence: sgRNA
  source 1..20: mol_type = other DNA
cgcttgaggctacaagtcca

SEQ ID NO: 257 - DNA, 20 bp, synthetic construct
  misc_feature 1..20: note = Gene: SH3BGRL
  misc_feature 1..20: note = Description of Sequence: sgRNA
  source 1..20: mol_type = other DNA
ccttaccgctgtagagccag

SEQ ID NO: 258 - DNA, 20 bp, synthetic construct
  misc_feature 1..20: note = Gene: SH3BGRL
  misc_feature 1..20: note = Description of Sequence: sgRNA
  source 1..20: mol_type = other DNA
ttgcagccaatgaagagaat

SEQ ID NO: 259 - DNA, 20 bp, synthetic construct
  misc_feature 1..20: note = Gene: SH3BGRL
  misc_feature 1..20: note = Description of Sequence: sgRNA
  source 1..20: mol_type = other DNA
aagaaacaacaagatgtgct

SEQ ID NO: 260 - DNA, 20 bp, synthetic construct
  misc_feature 1..20: note = Gene: SH3BGRL
  misc_feature 1..20: note = Description of Sequence: sgRNA
  source 1..20: mol_type = other DNA
gaaaatagtcgaccagccac

SEQ ID NO: 261 - DNA, 20 bp, synthetic construct
  misc_feature 1..20: note = Gene: SIT1
  misc_feature 1..20: note = Description of Sequence: sgRNA
  source 1..20: mol_type = other DNA
tggctgcacacttgtcccag

SEQ ID NO: 262 - DNA, 20 bp, synthetic construct
  misc_feature 1..20: note = Gene: SIT1
  misc_feature 1..20: note = Description of Sequence: sgRNA
  source 1..20: mol_type = other DNA
tgcattatctacagacaggt

SEQ ID NO: 263 - DNA, 20 bp, synthetic construct
  misc_feature 1..20: note = Gene: SIT1
  misc_feature 1..20: note = Description of Sequence: sgRNA
  source 1..20: mol_type = other DNA
aggaatcccctccataaccc

SEQ ID NO: 264 - DNA, 20 bp, synthetic construct
  misc_feature 1..20: note = Gene: SIT1
  misc_feature 1..20: note = Description of Sequence: sgRNA
  source 1..20: mol_type = other DNA
gagcagaggcgacaactgca

SEQ ID NO: 265 - DNA, 20 bp, synthetic construct
  misc_feature 1..20: note = Gene: SLC47A1
  misc_feature 1..20: note = Description of Sequence: sgRNA
  source 1..20: mol_type = other DNA
ttcataagctccgtgttctg

SEQ ID NO: 266 - DNA, 20 bp, synthetic construct
  misc_feature 1..20: note = Gene: SLC47A1
  misc_feature 1..20: note = Description of Sequence: sgRNA
  source 1..20: mol_type = other DNA
gaggtcgggagcttcctcag

SEQ ID NO: 267 - DNA, 20 bp, synthetic construct
  misc_feature 1..20: note = Gene: SLC47A1
  misc_feature 1..20: note = Description of Sequence: sgRNA
  source 1..20: mol_type = other DNA
aaactgcatcaagctacatg

SEQ ID NO: 268 - DNA, 20 bp, synthetic construct
  misc_feature 1..20: note = Gene: SLC47A1
  misc_feature 1..20: note = Description of Sequence: sgRNA
  source 1..20: mol_type = other DNA
gcaactccagttacgatctg

SEQ ID NO: 269 - DNA, 20 bp, synthetic construct
  misc_feature 1..20: note = Gene: SLC9A3
  misc_feature 1..20: note = Description of Sequence: sgRNA
  source 1..20: mol_type = other DNA
gaagaacactacgatgatgg

SEQ ID NO: 270 - DNA, 20 bp, synthetic construct
  misc_feature 1..20: note = Gene: SLC9A3
  misc_feature 1..20: note = Description of Sequence: sgRNA
  source 1..20: mol_type = other DNA
ctgtccggatatgtcctcga

SEQ ID NO: 271 - DNA, 20 bp, synthetic construct
  misc_feature 1..20: note = Gene: SLC9A3
  misc_feature 1..20: note = Description of Sequence: sgRNA
  source 1..20: mol_type = other DNA
actcacccatgagcccactg

SEQ ID NO: 272 - DNA, 20 bp, synthetic construct
  misc_feature 1..20: note = Gene: SLC9A3
  misc_feature 1..20: note = Description of Sequence: sgRNA
  source 1..20: mol_type = other DNA
gctgaacgacgcagtcaccg

SEQ ID NO: 273 - DNA, 20 bp, synthetic construct
  misc_feature 1..20: note = Gene: SP1
  misc_feature 1..20: note = Description of Sequence: sgRNA
  source 1..20: mol_type = other DNA
catcatccggacaccaacag

SEQ ID NO: 274 - DNA, 20 bp, synthetic construct
  misc_feature 1..20: note = Gene: SP1
  misc_feature 1..20: note = Description of Sequence: sgRNA
  source 1..20: mol_type = other DNA
gtatgtgaccaatgtaccag

SEQ ID NO: 275 - DNA, 20 bp, synthetic construct
  misc_feature 1..20: note = Gene: SP1
  misc_feature 1..20: note = Description of Sequence: sgRNA
  source 1..20: mol_type = other DNA
caacagattatcacaaatcg

SEQ ID NO: 276 - DNA, 20 bp, synthetic construct
  misc_feature 1..20: note = Gene: SP1
  misc_feature 1..20: note = Description of Sequence: sgRNA
  source 1..20: mol_type = other DNA
ttactaccagtggatcatca

SEQ ID NO: 277 - DNA, 20 bp, synthetic construct
  misc_feature 1..20: note = Gene: ST5
  misc_feature 1..20: note = Description of Sequence: sgRNA
  source 1..20: mol_type = other DNA
agcatgggaaggtcgccgag

SEQ ID NO: 278 - DNA, 20 bp, synthetic construct
  misc_feature 1..20: note = Gene: ST5
  misc_feature 1..20: note = Description of Sequence: sgRNA
  source 1..20: mol_type = other DNA
ctgtgcctcttgggtaatcg

SEQ ID NO: 279 - DNA, 20 bp, synthetic construct
  misc_feature 1..20: note = Gene: ST5
  misc_feature 1..20: note = Description of Sequence: sgRNA
  source 1..20: mol_type = other DNA
cgcccccagctatcgcacgc

SEQ ID NO: 280 - DNA, 20 bp, synthetic construct
  misc_feature 1..20: note = Gene: ST5
  misc_feature 1..20: note = Description of Sequence: sgRNA
  source 1..20: mol_type = other DNA
gaaggaggtagaccattact

SEQ ID NO: 281 - DNA, 20 bp, synthetic construct
  misc_feature 1..20: note = Gene: STAT6
  misc_feature 1..20: note = Description of Sequence: sgRNA
  source 1..20: mol_type = other DNA
gctggaaggcctccatactg

SEQ ID NO: 282 - DNA, 20 bp, synthetic construct
  misc_feature 1..20: note = Gene: STAT6
  misc_feature 1..20: note = Description of Sequence: sgRNA
  source 1..20: mol_type = other DNA
cagccaccacaaagggcacg

SEQ ID NO: 283 - DNA, 20 bp, synthetic construct
  misc_feature 1..20: note = Gene: STAT6
  misc_feature 1..20: note = Description of Sequence: sgRNA
  source 1..20: mol_type = other DNA
atcaagcggtgtgagcggaa

SEQ ID NO: 284 - DNA, 20 bp, synthetic construct
  misc_feature 1..20: note = Gene: STAT6
  misc_feature 1..20: note = Description of Sequence: sgRNA
  source 1..20: mol_type = other DNA
gctgggaataaatgtccacc

SEQ ID NO: 285 - DNA, 20 bp, synthetic construct
  misc_feature 1..20: note = Gene: TBL1Y
  misc_feature 1..20: note = Description of Sequence: sgRNA
  source 1..20: mol_type = other DNA
gggacagggactctataggg

SEQ ID NO: 286 - DNA, 20 bp, synthetic construct
  misc_feature 1..20: note = Gene: TBL1Y
  misc_feature 1..20: note = Description of Sequence: sgRNA
  source 1..20: mol_type = other DNA
aggctagcaaatcactgaca

SEQ ID NO: 287 - DNA, 20 bp, synthetic construct
  misc_feature 1..20: note = Gene: TBL1Y
  misc_feature 1..20: note = Description of Sequence: sgRNA
  source 1..20: mol_type = other DNA
gacattgtatacgagaaggg

SEQ ID NO: 288 - DNA, 20 bp, synthetic construct
  misc_feature 1..20: note = Gene: TBL1Y
  misc_feature 1..20: note = Description of Sequence: sgRNA
  source 1..20: mol_type = other DNA
aaatcctccaaagaaccgag

SEQ ID NO: 289 - DNA, 20 bp, synthetic construct
  misc_feature 1..20: note = Gene: TICRR
  misc_feature 1..20: note = Description of Sequence: sgRNA
  source 1..20: mol_type = other DNA
gacgctgctagactaccagt

SEQ ID NO: 290 - DNA, 20 bp, synthetic construct
  misc_feature 1..20: note = Gene: TICRR
  misc_feature 1..20: note = Description of Sequence: sgRNA
  source 1..20: mol_type = other DNA
ggatagagtcaatatacctg

SEQ ID NO: 291 - DNA, 20 bp, synthetic construct
  misc_feature 1..20: note = Gene: TICRR
  misc_feature 1..20: note = Description of Sequence: sgRNA
  source 1..20: mol_type = other DNA
tcaaagagatcactaaagcg

SEQ ID NO: 292 - DNA, 20 bp, synthetic construct
  misc_feature 1..20: note = Gene: TMEM62
  misc_feature 1..20: note = Description of Sequence: sgRNA
  source 1..20: mol_type = other DNA
caggaatccggtcaataatg

SEQ ID NO: 293 - DNA, 20 bp, synthetic construct
  misc_feature 1..20: note = Gene: TMEM62
  misc_feature 1..20: note = Description of Sequence: sgRNA
  source 1..20: mol_type = other DNA
atgaacgaatagttgccaaa

SEQ ID NO: 294 - DNA, 20 bp, synthetic construct
  misc_feature 1..20: note = Gene: TMEM62
  misc_feature 1..20: note = Description of Sequence: sgRNA
  source 1..20: mol_type = other DNA
gagactggaaggataatagg

SEQ ID NO: 295 - DNA, 20 bp, synthetic construct
  misc_feature 1..20: note = Gene: TMEM62
  misc_feature 1..20: note = Description of Sequence: sgRNA
  source 1..20: mol_type = other DNA
gtagaatggcaaacctacca

SEQ ID NO: 296 - DNA, 20 bp, synthetic construct
  misc_feature 1..20: note = Gene: TNK1
  misc_feature 1..20: note = Description of Sequence: sgRNA
  source 1..20: mol_type = other DNA
tgggcctaagtctaagaact

SEQ ID NO: 297 - DNA, 20 bp, synthetic construct
  misc_feature 1..20: note = Gene: TNK1
  misc_feature 1..20: note = Description of Sequence: sgRNA
  source 1..20: mol_type = other DNA
cccaaacatccacacgtccg

SEQ ID NO: 298 - DNA, 20 bp, synthetic construct
  misc_feature 1..20: note = Gene: TNK1
  misc_feature 1..20: note = Description of Sequence: sgRNA
  source 1..20: mol_type = other DNA
aatggtcgcaccttcaaagt

SEQ ID NO: 299 - DNA, 20 bp, synthetic construct
  misc_feature 1..20: note = Gene: TNK1
  misc_feature 1..20: note = Description of Sequence: sgRNA
  source 1..20: mol_type = other DNA
cctctgggatcagacacttg

SEQ ID NO: 300 - DNA, 20 bp, synthetic construct
  misc_feature 1..20: note = Gene: TNS2
  misc_feature 1..20: note = Description of Sequence: sgRNA
  source 1..20: mol_type = other DNA
gcagctgtagtcaggcatcg

SEQ ID NO: 301 - DNA, 20 bp, synthetic construct
  misc_feature 1..20: note = Gene: TNS2
  misc_feature 1..20: note = Description of Sequence: sgRNA
  source 1..20: mol_type = other DNA
gctgagctacgagatcccta

SEQ ID NO: 302 - DNA, 20 bp, synthetic construct
  misc_feature 1..20: note = Gene: TNS2
  misc_feature 1..20: note = Description of Sequence: sgRNA
  source 1..20: mol_type = other DNA
gtaacatgttatcacaaggg

SEQ ID NO: 303 - DNA, 20 bp, synthetic construct
  misc_feature 1..20: note = Gene: TNS2
  misc_feature 1..20: note = Description of Sequence: sgRNA
  source 1..20: mol_type = other DNA
ggtcgtactatactgcaagg

SEQ ID NO: 304 - DNA, 20 bp, synthetic construct
  misc_feature 1..20: note = Gene: TP53BP1
  misc_feature 1..20: note = Description of Sequence: sgRNA
  source 1..20: mol_type = other DNA
tcatgtgacgatgtaagaca

SEQ ID NO: 305 - DNA, 20 bp, synthetic construct
  misc_feature 1..20: note = Gene: TP53BP1
  misc_feature 1..20: note = Description of Sequence: sgRNA
  source 1..20: mol_type = other DNA
gaagcccattagtcctgtca

SEQ ID NO: 306 - DNA, 20 bp, synthetic construct
  misc_feature 1..20: note = Gene: TP53BP1
  misc_feature 1..20: note = Description of Sequence: sgRNA
  source 1..20: mol_type = other DNA
ggaatcaatactaatcacac

SEQ ID NO: 307 - DNA, 20 bp, synthetic construct
  misc_feature 1..20: note = Gene: TP53BP1
  misc_feature 1..20: note = Description of Sequence: sgRNA
  source 1..20: mol_type = other DNA
agacccatgatcccatactt

SEQ ID NO: 308 - DNA, 20 bp, synthetic construct
  misc_feature 1..20: note = Gene: TTN
  misc_feature 1..20: note = Description of Sequence: sgRNA
  source 1..20: mol_type = other DNA
agaacctgcaacaatcaccg

SEQ ID NO: 309 - DNA, 20 bp, synthetic construct
  misc_feature 1..20: note = Gene: TTN
  misc_feature 1..20: note = Description of Sequence: sgRNA
  source 1..20: mol_type = other DNA
gtacctaacgacgaaaggtg

SEQ ID NO: 310 - DNA, 20 bp, synthetic construct
  misc_feature 1..20: note = Gene: TTN
  misc_feature 1..20: note = Description of Sequence: sgRNA
  source 1..20: mol_type = other DNA
gtccttgtaggatagcaatg

SEQ ID NO: 311 - DNA, 20 bp, synthetic construct
  misc_feature 1..20: note = Gene: TTN
  misc_feature 1..20: note = Description of Sequence: sgRNA
  source 1..20: mol_type = other DNA
agaggttcaataaagtacgg

SEQ ID NO: 312 - DNA, 20 bp, synthetic construct
  misc_feature 1..20: note = Gene: TUFM
  misc_feature 1..20: note = Description of Sequence: sgRNA
  source 1..20: mol_type = other DNA
acaggcactgcacccctcga

SEQ ID NO: 313 - DNA, 20 bp, synthetic construct
  misc_feature 1..20: note = Gene: TUFM
  misc_feature 1..20: note = Description of Sequence: sgRNA
  source 1..20: mol_type = other DNA
agcagagcctacgatgactg

SEQ ID NO: 314 - DNA, 20 bp, synthetic construct
  misc_feature 1..20: note = Gene: TUFM
  misc_feature 1..20: note = Description of Sequence: sgRNA
  source 1..20: mol_type = other DNA
tcggggtatcaccatcaatg

SEQ ID NO: 315 - DNA, 20 bp, synthetic construct
  misc_feature 1..20: note = Gene: TUFM
  misc_feature 1..20: note = Description of Sequence: sgRNA
  source 1..20: mol_type = other DNA
gtgacaggtacactagagcg

SEQ ID NO: 316 - DNA, 20 bp, synthetic construct
  misc_feature 1..20: note = Gene: UPK1B
  misc_feature 1..20: note = Description of Sequence: sgRNA
  source 1..20: mol_type = other DNA
gcccacaaatatgccgatcc

SEQ ID NO: 317 - DNA, 20 bp, synthetic construct
  misc_feature 1..20: note = Gene: UPK1B
  misc_feature 1..20: note = Description of Sequence: sgRNA
  source 1..20: mol_type = other DNA
tgctgacaggacaattgctg

SEQ ID NO: 318 - DNA, 20 bp, synthetic construct
  misc_feature 1..20: note = Gene: UPK1B
  misc_feature 1..20: note = Description of Sequence: sgRNA
  source 1..20: mol_type = other DNA
ttgcggcattgccctgactg

SEQ ID NO: 319 - DNA, 20 bp, synthetic construct
  misc_feature 1..20: note = Gene: UPK1B
  misc_feature 1..20: note = Description of Sequence: sgRNA
  source 1..20: mol_type = other DNA
ctccaaacaatgatgaccag

SEQ ID NO: 320 - DNA, 20 bp, synthetic construct
  misc_feature 1..20: note = Gene: UQCRC1
  misc_feature 1..20: note = Description of Sequence: sgRNA
  source 1..20: mol_type = other DNA
aaggtagagcatcatcacgg

SEQ ID NO: 321 - DNA, 20 bp, synthetic construct
  misc_feature 1..20: note = Gene: UQCRC1
  misc_feature 1..20: note = Description of Sequence: sgRNA
  source 1..20: mol_type = other DNA
atgtccatgggatgccaccg

SEQ ID NO: 322 - DNA, 20 bp, synthetic construct
  misc_feature 1..20: note = Gene: UQCRC1
  misc_feature 1..20: note = Description of Sequence: sgRNA
  source 1..20: mol_type = other DNA
caccgtgcaagtgggctgag

SEQ ID NO: 323 - DNA, 20 bp, synthetic construct
  misc_feature 1..20: note = Gene: UQCRC1
  misc_feature 1..20: note = Description of Sequence: sgRNA
  source 1..20: mol_type = other DNA
gggccttgtaatgtgtgctg

SEQ ID NO: 324 - DNA, 20 bp, synthetic construct
  misc_feature 1..20: note = Gene: WWOX
  misc_feature 1..20: note = Description of Sequence: sgRNA
  source 1..20: mol_type = other DNA
caaggtagaagcaatgaccc

SEQ ID NO: 325 - DNA, 20 bp, synthetic construct
  misc_feature 1..20: note = Gene: WWOX
  misc_feature 1..20: note = Description of Sequence: sgRNA
  source 1..20: mol_type = other DNA
acaccgaggagaagactcag

SEQ ID NO: 326 - DNA, 20 bp, synthetic construct
  misc_feature 1..20: note = Gene: WWOX
  misc_feature 1..20: note = Description of Sequence: sgRNA
  source 1..20: mol_type = other DNA
ccaagatcacatgtgcacca

SEQ ID NO: 327 - DNA, 20 bp, synthetic construct
  misc_feature 1..20: note = Gene: WWOX
  misc_feature 1..20: note = Description of Sequence: sgRNA
  source 1..20: mol_type = other DNA
gccgtcgtatctttgccggg

SEQ ID NO: 328 - DNA, 20 bp, synthetic construct
  misc_feature 1..20: note = Gene: XCL1
  misc_feature 1..20: note = Description of Sequence: sgRNA
  source 1..20: mol_type = other DNA
gagacttcactccctacacc

SEQ ID NO: 329 - DNA, 20 bp, synthetic construct
  misc_feature 1..20: note = Gene: XCL1
  misc_feature 1..20: note = Description of Sequence: sgRNA
  source 1..20: mol_type = other DNA
tgctgatccacaagccacat

SEQ ID NO: 330 - DNA, 20 bp, synthetic construct
  misc_feature 1..20: note = Gene: XCL1
  misc_feature 1..20: note = Description of Sequence: sgRNA
  source 1..20: mol_type = other DNA
tctgctaaccggcagtcgct

SEQ ID NO: 331 - DNA, 20 bp, synthetic construct
  misc_feature 1..20: note = Gene: XCL1
  misc_feature 1..20: note = Description of Sequence: sgRNA
  source 1..20: mol_type = other DNA
ggagtgaagtctcagataag

SEQ ID NO: 332 - DNA, 20 bp, synthetic construct
  misc_feature 1..20: note = Gene: ZBTB7A
  misc_feature 1..20: note = Description of Sequence: sgRNA
  source 1..20: mol_type = other DNA
accgtcagcacagccaacgt

SEQ ID NO: 333 - DNA, 20 bp, synthetic construct
  misc_feature 1..20: note = Gene: ZBTB7A
  misc_feature 1..20: note = Description of Sequence: sgRNA
  source 1..20: mol_type = other DNA
gagtcgcgggccgacgacaa

SEQ ID NO: 334 - DNA, 20 bp, synthetic construct
  misc_feature 1..20: note = Gene: ZBTB7A
  misc_feature 1..20: note = Description of Sequence: sgRNA
  source 1..20: mol_type = other DNA
gccgtagtggccgttctgcg

SEQ ID NO: 335 - DNA, 20 bp, synthetic construct
  misc_feature 1..20: note = Gene: ZBTB7A
  misc_feature 1..20: note = Description of Sequence: sgRNA
  source 1..20: mol_type = other DNA
atcatcggacgccccaaagg

SEQ ID NO: 336 - DNA, 20 bp, synthetic construct
  misc_feature 1..20: note = Gene: ZFYVE28
  misc_feature 1..20: note = Description of Sequence: sgRNA
  source 1..20: mol_type = other DNA
cccccacaagcttagcactg

SEQ ID NO: 337 - DNA, 20 bp, synthetic construct
  misc_feature 1..20: note = Gene: ZFYVE28
  misc_feature 1..20: note = Description of Sequence: sgRNA
  source 1..20: mol_type = other DNA
atgtgtttgcagctacgtct

SEQ ID NO: 338 - DNA, 20 bp, synthetic construct
  misc_feature 1..20: note = Gene: ZFYVE28
  misc_feature 1..20: note = Description of Sequence: sgRNA
  source 1..20: mol_type = other DNA
cttgagcaacaacaatctcg

SEQ ID NO: 339 - DNA, 20 bp, synthetic construct
  misc_feature 1..20: note = Gene: ZFYVE28
  misc_feature 1..20: note = Description of Sequence: sgRNA
  source 1..20: mol_type = other DNA
ttgctgcggaaaataaggtg

SEQ ID NO: 340 - DNA, 20 bp, synthetic construct
  misc_feature 1..20: note = Gene: ZNF101
  misc_feature 1..20: note = Description of Sequence: sgRNA
  source 1..20: mol_type = other DNA
catgaaagaactcatagtgg

SEQ ID NO: 341 - DNA, 20 bp, synthetic construct
  misc_feature 1..20: note = Gene: ZNF101
  misc_feature 1..20: note = Description of Sequence: sgRNA
  source 1..20: mol_type = other DNA
cccgtaaacagaaacaacat

SEQ ID NO: 342 - DNA, 20 bp, synthetic construct
  misc_feature 1..20: note = Gene: ZNF101
  misc_feature 1..20: note = Description of Sequence: sgRNA
  source 1..20: mol_type = other DNA
tgaaatcagatctcacgcgc

SEQ ID NO: 343 - DNA, 20 bp, synthetic construct
  misc_feature 1..20: note = Gene: ZNF101
  misc_feature 1..20: note = Description of Sequence: sgRNA
  source 1..20: mol_type = other DNA
aataaactgggataatcgat

SEQ ID NO: 344 - DNA, 20 bp, synthetic construct
  misc_feature 1..20: note = Gene: ZNF436
  misc_feature 1..20: note = Description of Sequence: sgRNA
  source 1..20: mol_type = other DNA
ctcacctaatccaacaccaa

SEQ ID NO: 345 - DNA, 20 bp, synthetic construct
  misc_feature 1..20: note = Gene: ZNF436
  misc_feature 1..20: note = Description of Sequence: sgRNA
  source 1..20: mol_type = other DNA
gataggtcagaaagacaatg

SEQ ID NO: 346 - DNA, 20 bp, synthetic construct
  misc_feature 1..20: note = Gene: ZNF436
  misc_feature 1..20: note = Description of Sequence: sgRNA
  source 1..20: mol_type = other DNA
tgagatcaggagtgagaacg

SEQ ID NO: 347 - DNA, 20 bp, synthetic construct
  misc_feature 1..20: note = Gene: ZNF436
  misc_feature 1..20: note = Description of Sequence: sgRNA
  source 1..20: mol_type = other DNA
aatgagaacatatatgatag

SEQ ID NO: 348 - DNA, 20 bp, synthetic construct
  misc_feature 1..20: note = Gene: ZNF506
  misc_feature 1..20: note = Description of Sequence: sgRNA
  source 1..20: mol_type = other DNA
aaaaacctttaactatgaag

SEQ ID NO: 349 - DNA, 20 bp, synthetic construct
  misc_feature 1..20: note = Gene: ZNF506
  misc_feature 1..20: note = Description of Sequence: sgRNA
  source 1..20: mol_type = other DNA
gagtgtccagtgcacaaaag

SEQ ID NO: 350 - DNA, 20 bp, synthetic construct
  misc_feature 1..20: note = Gene: ZNF506
  misc_feature 1..20: note = Description of Sequence: sgRNA
  source 1..20: mol_type = other DNA
tatgtgaaaaaagggttgca

SEQ ID NO: 351 - DNA, 20 bp, synthetic construct
  misc_feature 1..20: note = Gene: ZNF716
  misc_feature 1..20: note = Description of Sequence: sgRNA
  source 1..20: mol_type = other DNA
catttcaaatgtaaaaacga

SEQ ID NO: 352 - DNA, 20 bp, synthetic construct
  misc_feature 1..20: note = Gene: ZNF716
  misc_feature 1..20: note = Description of Sequence: sgRNA
  source 1..20: mol_type = other DNA
caagaccttcagtcagagca

SEQ ID NO: 353 - DNA, 20 bp, synthetic construct
  misc_feature 1..20: note = Gene: ZNF716
  misc_feature 1..20: note = Description of Sequence: sgRNA
  source 1..20: mol_type = other DNA
ggttagtaagtgttgaagag

SEQ ID NO: 354 - DNA, 20 bp, synthetic construct
  misc_feature 1..20: note = Gene: ZNF716
  misc_feature 1..20: note = Description of Sequence: sgRNA
  source 1..20: mol_type = other DNA
tgtagttagtaagtgttgag

SEQ ID NO: 355 - DNA, 20 bp, synthetic construct
  misc_feature 1..20: note = Gene: ZNF805
  misc_feature 1..20: note = Description of Sequence: sgRNA
  source 1..20: mol_type = other DNA
gtccatgactgtgactcaca

SEQ ID NO: 356 - DNA, 20 bp, synthetic construct
  misc_feature 1..20: note = Gene: ZNF805
  misc_feature 1..20: note = Description of Sequence: sgRNA
  source 1..20: mol_type = other DNA
gccacattcgaagcactcgt

SEQ ID NO: 357 - DNA, 20 bp, synthetic construct
  misc_feature 1..20: note = Gene: ZNF805
  misc_feature 1..20: note = Description of Sequence: sgRNA
  source 1..20: mol_type = other DNA
aaaagagattccttcagaca

SEQ ID NO: 358 - DNA, 20 bp, synthetic construct
  misc_feature 1..20: note = Gene: ZNF805
  misc_feature 1..20: note = Description of Sequence: sgRNA
  source 1..20: mol_type = other DNA
agggagaacgcttgagacca

SEQ ID NO: 359 - DNA, 20 bp, synthetic construct
  misc_feature 1..20: note = Description of Sequence: sgRNA
  misc_feature 1..20: note = Type: CGS
  misc_feature 1..20: note = Name: ADORA2A_g1
  source 1..20: mol_type = other DNA
atgctaggttggaacaactg

SEQ ID NO: 360 - RNA, 20 nt, synthetic construct
  misc_feature 1..20: note = Description of Sequence: sgRNA
  misc_feature 1..20: note = Type: CGS
  misc_feature 1..20: note = Name: ADORA2A_g2
  source 1..20: mol_type = other RNA
cgaggagcccatgatgggca

SEQ ID NO: 361 - DNA, 20 bp, synthetic construct
  misc_feature 1..20: note = Description of Sequence: sgRNA
  misc_feature 1..20: note = Type: CGS
  misc_feature 1..20: note = Name: FAM105A_g1
  source 1..20: mol_type = other DNA
gaagtgaccaagttcactcc

SEQ ID NO: 362 - RNA, 20 nt, synthetic construct
  misc_feature 1..20: note = Description of Sequence: sgRNA
  misc_feature 1..20: note = Type: CGS
  misc_feature 1..20: note = Name: FAM105A_g2
  source 1..20: mol_type = other RNA
ctagcatccaggagtgaact

SEQ ID NO: 363 - DNA, 20 bp, synthetic construct
  misc_feature 1..20: note = Description of Sequence: sgRNA
  misc_feature 1..20: note = Type: CGS
  misc_feature 1..20: note = Name: NKX2-6_g1
  source 1..20: mol_type = other DNA
tttagagcccggcctgaacg

SEQ ID NO: 364 - RNA, 20 nt, synthetic construct
  misc_feature 1..20: note = Description of Sequence: sgRNA
  misc_feature 1..20: note = Type: CGS
  misc_feature 1..20: note = Name: NKX2-6_g2
  source 1..20: mol_type = other RNA
gtccttgaccgagaaggggg

SEQ ID NO: 365 - DNA, 20 bp, synthetic construct
  misc_feature 1..20: note = Description of Sequence: sgRNA
  misc_feature 1..20: note = Type: CGS
  misc_feature 1..20: note = Name: PDE4C_g1
  source 1..20: mol_type = other DNA
ctagaagacaccaacaagtg

SEQ ID NO: 366 - RNA, 20 nt, synthetic construct
  misc_feature 1..20: note = Description of Sequence: sgRNA
  misc_feature 1..20: note = Type: CGS
  misc_feature 1..20: note = Name: PDE4C_g2
  source 1..20: mol_type = other RNA
ctctcctttcagctttgacc

SEQ ID NO: 367 - DNA, 20 bp, synthetic construct
  misc_feature 1..20: note = Description of Sequence: sgRNA
  misc_feature 1..20: note = Type: CGS
  misc_feature 1..20: note = Name: PLXNA4_g1
  source 1..20: mol_type = other DNA
gcggtatggactctccacgc

SEQ ID NO: 368 - RNA, 20 nt, synthetic construct
  misc_feature 1..20: note = Description of Sequence: sgRNA
  misc_feature 1..20: note = Type: CGS
  misc_feature 1..20: note = Name: PLXNA4_g2
  source 1..20: mol_type = other RNA
gcaggtccagttccagggca

SEQ ID NO: 369 - DNA, 19 bp, synthetic construct
  misc_feature 1..19: note = Description of Sequence: sgRNA
  misc_feature 1..19: note = Type: CTRL
  misc_feature 1..19: note = Name: AAVS1_g1
  source 1..19: mol_type = other DNA
gggccactagggacaggat

SEQ ID NO: 370 - DNA, 20 bp, synthetic construct
  misc_feature 1..20: note = Description of Sequence: sgRNA
  misc_feature 1..20: note = Type: CTRL
  misc_feature 1..20: note = Name: AAVS1_g2
  source 1..20: mol_type = other DNA
gtcaccaatcctgtccctag

SEQ ID NO: 371 - DNA, 20 bp, synthetic construct
  misc_feature 1..20: note = Description of Sequence: sgRNA
  misc_feature 1..20: note = Type: CYC
  misc_feature 1..20: note = Name: MOB3C_g1
  source 1..20: mol_type = other DNA
agtgtggtgaggctaccacc

SEQ ID NO: 372 - RNA, 20 nt, synthetic construct
  misc_feature 1..20: note = Description of Sequence: sgRNA
  misc_feature 1..20: note = Type: CYC
  misc_feature 1..20: note = Name: MOB3C_g2
  source 1..20: mol_type = other RNA
ctgcttcaggcacagggcca

SEQ ID NO: 373 - DNA, 20 bp, synthetic construct
  misc_feature 1..20: note = Description of Sequence: sgRNA
  misc_feature 1..20: note = Type: CYC
  misc_feature 1..20: note = Name: NFKB2_g1
  source 1..20: mol_type = other DNA
gggaccagccaagatcgagg

SEQ ID NO: 374 - RNA, 20 nt, synthetic construct
  misc_feature 1..20: note = Description of Sequence: sgRNA
  misc_feature 1..20: note = Type: CYC
  misc_feature 1..20: note = Name: NFKB2_g2
  source 1..20: mol_type = other RNA
tccctcgtagttacagatct

SEQ ID NO: 375 - DNA, 20 bp, synthetic construct
  misc_feature 1..20: note = Description of Sequence: sgRNA
  misc_feature 1..20: note = Type: CYC
  misc_feature 1..20: note = Name: NFKBIA_g1
  source 1..20: mol_type = other DNA
ggttggtgatcacagccaag

SEQ ID NO: 376 - RNA, 20 nt, synthetic construct
  misc_feature 1..20: note = Description of Sequence: sgRNA
  misc_feature 1..20: note = Type: CYC
  misc_feature 1..20: note = Name: NFKBIA_g2
  source 1..20: mol_type = other RNA
catggatgatggccaagtgc

SEQ ID NO: 377 - DNA, 20 bp, synthetic construct
  misc_feature 1..20: note = Description of Sequence: sgRNA
  misc_feature 1..20: note = Type: CYC
  misc_feature 1..20: note = Name: PIM2_g1
  source 1..20: mol_type = other DNA
caagcaggcggatcacgcca

SEQ ID NO: 378 - RNA, 20 nt, synthetic construct
  misc_feature 1..20: note = Description of Sequence: sgRNA
  misc_feature 1..20: note = Type: CYC
  misc_feature 1..20: note = Name: PIM2_g2
  source 1..20: mol_type = other RNA
ctccgcctgcgtaggaggca

SEQ ID NO: 379 - DNA, 20 bp, synthetic construct
  misc_feature 1..20: note = Description of Sequence: sgRNA
  misc_feature 1..20: note = Type: CYC
  misc_feature 1..20: note = Name: PPIA_g1
  source 1..20: mol_type = other DNA
cgccgcccgcccgacctcaa

SEQ ID NO: 380 - RNA, 20 nt, synthetic construct
  misc_feature 1..20: note = Description of Sequence: sgRNA
  misc_feature 1..20: note = Type: CYC
  misc_feature 1..20: note = Name: PPIA_g2
  source 1..20: mol_type = other RNA
gtacccttaccactcagtct

SEQ ID NO: 381 - DNA, 20 bp, synthetic construct
  misc_feature 1..20: note = Description of Sequence: sgRNA
  misc_feature 1..20: note = Type: PAN
  misc_feature 1..20: note = Name: CBLB_g1
  source 1..20: mol_type = other DNA
tgcacagaactatcgtacca

SEQ ID NO: 382 - RNA, 20 nt, synthetic construct
  misc_feature 1..20: note = Description of Sequence: sgRNA
  misc_feature 1..20: note = Type: PAN
  misc_feature 1..20: note = Name: CBLB_g2
  source 1..20: mol_type = other RNA
aaatatcaagtatatatggt

SEQ ID NO: 383 - DNA, 20 bp, synthetic construct
  misc_feature 1..20: note = Description of Sequence: sgRNA
  misc_feature 1..20: note = Type: PAN
  misc_feature 1..20: note = Name: CYC1_g1
  source 1..20: mol_type = other DNA
ggtgggcgtgtgctacacgg

SEQ ID NO: 384 - RNA, 20 nt, synthetic construct
  misc_feature 1..20: note = Description of Sequence: sgRNA
  misc_feature 1..20: note = Type: PAN
  misc_feature 1..20: note = Name: CYC1_g2
  source 1..20: mol_type = other RNA
agcggagatcttgcaggcag

SEQ ID NO: 385 - DNA, 20 bp, synthetic construct
  misc_feature 1..20: note = Description of Sequence: sgRNA
  misc_feature 1..20: note = Type: PAN
  misc_feature 1..20: note = Name: FAM49B_g1
  source 1..20: mol_type = other DNA
tatgaggattaacaatgtac

SEQ ID NO: 386 - RNA, 20 nt, synthetic construct
  misc_feature 1..20: note = Description of Sequence: sgRNA
  misc_feature 1..20: note = Type: PAN
  misc_feature 1..20: note = Name: FAM49B_g2
  source 1..20: mol_type = other RNA
cttctcagactctgtaggct

SEQ ID NO: 387 - DNA, 20 bp, synthetic construct
  misc_feature 1..20: note = Description of Sequence: sgRNA
  misc_feature 1..20: note = Type: PAN
  misc_feature 1..20: note = Name: RASA2_g1
  source 1..20: mol_type = other DNA
agatatcacacattacagtg

SEQ ID NO: 388 - RNA, 20 nt, synthetic construct
  misc_feature 1..20: note = Description of Sequence: sgRNA
  misc_feature 1..20: note = Type: PAN
  misc_feature 1..20: note = Name: RASA2_g2
  source 1..20: mol_type = other RNA
attttgtggggtccaagata

SEQ ID NO: 389 - DNA, 20 bp, synthetic construct
  misc_feature 1..20: note = Description of Sequence: sgRNA
  misc_feature 1..20: note = Type: PAN
  misc_feature 1..20: note = Name: TMEM222_g1
  source 1..20: mol_type = other DNA
acggacatgaagcaatatca

SEQ ID NO: 390 - RNA, 20 nt, synthetic construct
  misc_feature 1..20: note = Description of Sequence: sgRNA
  misc_feature 1..20: note = Type: PAN
  misc_feature 1..20: note = Name: TMEM222_g2
  source 1..20: mol_type = other RNA
tgtcacagcagagattgtgc

SEQ ID NO: 391 - RNA, 20 nt, synthetic construct
  misc_feature 1..20: note = Description of Sequence: sgRNA
  misc_feature 1..20: note = Type: TAC
  misc_feature 1..20: note = Name: FKBP1A_g1
  source 1..20: mol_type = other RNA
ttcacagggatgcttgaaga

SEQ ID NO: 392 - RNA, 20 nt, synthetic construct
  misc_feature 1..20: note = Description of Sequence: sgRNA
  misc_feature 1..20: note = Type: TAC
  misc_feature 1..20: note = Name: FKBP1A_g2
  source 1..20: mol_type = other RNA
ctgggaagaaggggttgccc

SEQ ID NO: 393 - DNA, 20 bp, synthetic construct
  misc_feature 1..20: note = Description of Sequence: sgRNA
  misc_feature 1..20: note = Type: PAN
  misc_feature 1..20: note = Name: PFN1_g1
  source 1..20: mol_type = other DNA
gatcttcgtaccaagagcac

SEQ ID NO: 394 - RNA, 20 nt, synthetic construct
  misc_feature 1..20: note = Description of Sequence: sgRNA
  misc_feature 1..20: note = Type: PAN
  misc_feature 1..20: note = Name: PFN1_g2
  source 1..20: mol_type = other RNA
gttcctcttccagccagctg

SEQ ID NO: 395 - DNA, 20 bp, synthetic construct
  misc_feature 1..20: note = Description of Sequence: sgRNA
  misc_feature 1..20: note = Type: TAC
  misc_feature 1..20: note = Name: TGIF2_g1
  source 1..20: mol_type = other DNA
ggagtcggtgaagatcctcc

SEQ ID NO: 396 - RNA, 20 nt, synthetic construct
  misc_feature 1..20: note = Description of Sequence: sgRNA
  misc_feature 1..20: note = Type: TAC
  misc_feature 1..20: note = Name: TGIF2_g2
  source 1..20: mol_type = other RNA
ctagcccctaggcaccatgt

SEQ ID NO: 397 - DNA, 20 bp, synthetic construct
  misc_feature 1..20: note = Description of Sequence: sgRNA
  misc_feature 1..20: note = Type: TGFB
  misc_feature 1..20: note = Name: GTF2I_g1
  source 1..20: mol_type = other DNA
caacatgagactggaaaaga

SEQ ID NO: 398 - RNA, 20 nt, synthetic construct
  misc_feature 1..20: note = Description of Sequence: sgRNA
  misc_feature 1..20: note = Type: TGFB
  misc_feature 1..20: note = Name: GTF2I_g2
  source 1..20: mol_type = other RNA
tgtattgtagtgtaaagaac

SEQ ID NO: 399 - DNA, 20 bp, synthetic construct
  misc_feature 1..20: note = Description of Sequence: sgRNA
  misc_feature 1..20: note = Type: TGFB
  misc_feature 1..20: note = Name: LHX8_g1
  source 1..20: mol_type = other DNA
gacaccggacatgccagcat

SEQ ID NO: 400 - RNA, 20 nt, synthetic construct
  misc_feature 1..20: note = Description of Sequence: sgRNA
  misc_feature 1..20: note = Type: TGFB
  misc_feature 1..20: note = Name: LHX8_g2
  source 1..20: mol_type = other RNA
ttcctgcaggtgagccccga

SEQ ID NO: 401 - DNA, 20 bp, synthetic construct
  misc_feature 1..20: note = Description of Sequence: sgRNA
  misc_feature 1..20: note = Type: TGFB
  misc_feature 1..20: note = Name: SIRT7_g1
  source 1..20: mol_type = other DNA
gcgtctatcccagactaccg

SEQ ID NO: 402 - RNA, 20 nt, synthetic construct
  misc_feature 1..20: note = Description of Sequence: sgRNA
  misc_feature 1..20: note = Type: TGFB
  misc_feature 1..20: note = Name: SIRT7_g2
  source 1..20: mol_type = other RNA
cgcaggtgtcgcgcatcctg

SEQ ID NO: 403 - DNA, 20 bp, synthetic construct
  misc_feature 1..20: note = Description of Sequence: sgRNA
  misc_feature 1..20: note = Type: TGFB
  misc_feature 1..20: note = Name: TGFBR1_g1
  source 1..20: mol_type = other DNA
taaaagggcgatctaatgaa

SEQ ID NO: 404 - RNA, 20 nt, synthetic construct
  misc_feature 1..20: note = Description of Sequence: sgRNA
  misc_feature 1..20: note = Type: TGFB
  misc_feature 1..20: note = Name: TGFBR1_g2
  source 1..20: mol_type = other RNA
tggcagaaacactgtaacgc

SEQ ID NO: 405 - DNA, 20 bp, synthetic construct
  misc_feature 1..20: note = Description of Sequence: sgRNA
  misc_feature 1..20: note = Type: TGFB
  misc_feature 1..20: note = Name: TGFBR2_g1
  source 1..20: mol_type = other DNA
gcagaagctgagttcaacct

SEQ ID NO: 406 - RNA, 20 nt, synthetic construct
  misc_feature 1..20: note = Description of Sequence: sgRNA
  misc_feature 1..20: note = Type: TGFB
  misc_feature 1..20: note = Name: TGFBR2_g2
  source 1..20: mol_type = other RNA
tatcatgtcgttattaactg